(19) United States
(12) Patent Application Publication
(10) Pub. No.: US 2022/0073875 A1
(43) Pub. Date: Mar. 10, 2022

(54) Amplifiable RNAs for Therapeutic Cell Systems

(71) Applicant: Rubius Therapeutics, Inc., Cambridge, MA (US)

(72) Inventors: Qin Yu, Lexington, MA (US); Urjeet Khanwalkar, Cambridge, MA (US); Omid Harandi, Newton, MA (US); Ewan Dunn, Beverly, MA (US); Tanyaporn Pattarabanjird, Charlottesville, VA (US); Avak Kahvejian, Lexington, MA (US); Jordi Mata-Fink, Baltimore, MD (US)

(73) Assignee: Rubius Therapeutics, Inc., Cambridge, MA (US)

(21) Appl. No.: 17/529,686

(22) Filed: Nov. 18, 2021

Related U.S. Application Data
(63) Continuation of application No. 16/245,056, filed on Jan. 10, 2019.
(60) Provisional application No. 62/615,834, filed on Jan. 10, 2018.

(51) Int. Cl. C12N 5/078; C07K 14/18; C12N 15/113; C12N 15/86; C12N 15/69; C12N 5/0789

(52) U.S. Cl. CPC C12N 5/0634; C07K 14/1825; C12N 5/0641; A61K 45/06; C12N 15/86; C12N 15/69; C12N 5/0647; C12N 15/113

(57) ABSTRACT

This disclosure provides, among other things, amplifiable nucleic acid constructs for expressing a gene of interest in a cell, e.g., an erythroid cell. The amplifiable nucleic acid construct may contain the gene of interest and an RNA-dependent RNA polymerase (RdRP)-responsive 5′ UTR, and may optionally further contain an RdRP-responsive 3′ UTR. RdRP may also be provided, e.g., on the same construct or a different construct.

### RELATED APPLICATIONS

This application claims priority to U.S. Ser. No. 62/615,834 filed Jan. 10, 2018, the contents of which are incorporated herein by reference in their entirety.

### BACKGROUND

Flaviviruses have an RNA genome that includes several structural genes, e.g., to form the capsid and envelope that encapsulate the viral genome. In addition to several non-structural proteins, flavivirus genomes encode an RNA-dependent RNA polymerase (RdRP) that specifically replicates the viral genome, in a process that involves the recognition of specific structures at the 5′ untranslated region (UTR) and the 3′ UTR of the genome. Flavivirus-derived sequences are not commonly used in vectors to direct expression of transgenes.

The ability to express exogenous proteins in a variety of cell types (e.g., mammalian, bacterial and plant cells) have led to a multitude of commercial and therapeutic applications. For example, erythroid cells can be engineered to produce a wide variety of exogenous therapeutic proteins in order to treat different diseases. Generally, erythroid cell engineering can involve introducing a transgene into erythroid cell precursors, and inducing the precursors to differentiate and express the transgene. Although multiple systems and methods for introducing transgenes into cells have been developed, improved systems and methods that allow for the controlled amplification of transgenes and/or expression of exogenous proteins are desired.

### SUMMARY OF THE INVENTION

This disclosure provides, among other things, compositions and methods that can be used for producing a protein of interest (e.g., a therapeutic agent) in a cell, such as an erythroid cell. Generally, the present invention features nucleic acid constructs, e.g., DNA or RNA constructs, that comprise a gene of interest (e.g., encoding an exogenous protein of interest, e.g., a therapeutic agent). The nucleic acid constructs are capable of being replicated in the cell, thereby generating multiple copies of the nucleic acid construct that serve as a template for expressing the exogenous protein of interest. In some instances, a nucleic acid construct comprising an RNA-dependent RNA polymerase (RdRP)-responsive 5′ UTR (e.g., a flavivirus 5′ UTR, e.g., as described herein) and a gene encoding a protein of interest (e.g., a therapeutic agent) is introduced into an erythroid cell precursor. The erythroid cell precursor also comprises an RdRP that specifically binds to the RdRP-responsive 5′ UTR, and synthesizes an RNA (e.g., a positive strand RNA or a negative strand RNA) from the initial RNA construct. Further rounds of replication can create a PCR-like exponential amplification of the nucleic acid construct inside the cell. The protein (e.g., therapeutic agent) encoded by the nucleic acid construct can then be expressed at high levels.

The present disclosure provides, in some aspects, a cell, e.g., an erythroid cell (e.g., a nucleated erythroid cell precursor or an enucleated erythroid cell), comprising a nucleic acid molecule (e.g., RNA or DNA) comprising an RdRP-responsive 5′ UTR (e.g., a flavivirus 5′ UTR) and an exogenous nucleic acid sequence comprising an exogenous gene (e.g., a non-flaviviral gene).

The present disclosure also provides, in some aspects, a cell, e.g., an enucleated erythroid cell comprising an exogenous non-flaviviral protein or noncoding RNA, wherein the exogenous non-flaviviral protein or noncoding RNA was produced in or by a precursor of the enucleated erythroid cell from a nucleic acid molecule (e.g., RNA or DNA) comprising an RdRP-responsive 5′ UTR (e.g., a flavivirus 5′ UTR) and an exogenous nucleic acid sequence comprising a non-flaviviral gene encoding the exogenous non-flaviviral protein or noncoding RNA.

The present disclosure also provides, in some aspects, a nucleic acid construct (e.g., DNA or RNA construct) comprising an RdRP-responsive 5′ UTR (e.g., a flavivirus 5′ UTR) and an exogenous nucleic acid sequence comprising a non-flaviviral gene, wherein the nucleic acid construct does not comprise any sequences encoding flavivirus structural proteins, e.g., flavivirus C, prM, or E protein, or any combination thereof.

The present disclosure also provides, in some aspects, a nucleic acid construct (e.g., DNA or RNA construct) comprising an RdRP-responsive 5′ UTR (e.g., a flavivirus 5′ UTR) and an exogenous nucleic acid sequence comprising a non-flaviviral gene, wherein the nucleic acid construct does not comprise any sequences encoding flavivirus nonstructural proteins, e.g., genes encoding flavivirus NS1, NS2A, NS2B, NS3, NS4A, NS4B, or NS5 proteins.

The present disclosure also provides, in some aspects, a nucleic acid construct (e.g., DNA or RNA construct) comprising an RdRP-responsive 5′ UTR (e.g., a flavivirus 5′ UTR) and an exogenous nucleic acid sequence comprising a non-flaviviral gene, wherein the nucleic acid construct does not comprise any sequences encoding flavivirus NS1, NS2A, NS2B, NS3, NS4A, or NS4B proteins.

The present disclosure also provides, in some aspects, a nucleic acid construct (e.g., DNA or RNA construct) comprising an RdRP-responsive 5′ UTR (e.g., a flavivirus 5′ UTR) and lacking at least a portion of a gene encoding a flavivirus nonstructural protein, optionally wherein the nucleic acid construct (e.g., the RNA construct) further comprises an exogenous nucleic acid sequence comprising a non-flaviviral gene.

The present disclosure also provides, in some aspects, a nucleic acid construct (e.g., DNA or RNA construct) comprising an RdRP-responsive 5′ UTR (e.g., a flavivirus 5′ UTR) sequence and an exogenous gene selected from:
a) exogenous primate gene, e.g., a human gene;
b) a single-chain variable fragment (scFv);
c) a non-viral noncoding RNA. e.g., a microRNA, ribozyme, aptamer, siRNA, piRNA, snoRNA, snRNA, long ncRNA, or tRNA;
d) a gene encoding a non-viral protein that is not a reporter, e.g., a nonviral protein that is not a fluorescent protein, GFP, β-galactosidase, or chloramphenicol acetyltransferase (CAT); or
e) a gene encoding a fusion protein which comprises a first domain (e.g., a non-flaviviral protein, or a portion thereof) and a transmembrane domain. e.g., a Type I, Type II, or Type III transmembrane domain.

The present disclosure also provides, in some aspects, a composition comprising:
(i) a first nucleic acid molecule (e.g., RNA molecule) comprising an RdRP-responsive 5′ UTR (e.g., a flavivirus 5′ UTR) and an exogenous nucleic acid sequence (e.g., a non-viral sequence); wherein the first nucleic acid molecule does not comprise a sequence encoding a functional RNA-dependent RNA polymerase (RdRP), and
(ii) a second nucleic acid molecule (e.g., RNA molecule) comprising a sequence encoding a functional RdRP;
wherein the first nucleic acid molecule and the second nucleic acid molecule are each non-naturally occurring nucleic acid molecules. In some embodiments, the composition further comprises a third nucleic acid molecule comprising a nucleic acid sequence encoding a helicase.

The present disclosure also provides, in some aspects, a composition comprising:
(i) a first nucleic acid molecule (e.g., RNA molecule) comprising an RdRP-responsive 5′ UTR (e.g., a flavivirus 5′ UTR) and a first exogenous nucleic acid sequence (e.g., a gene of interest, e.g., encoding an exogenous protein, e.g., a therapeutic protein); and
(ii) a second nucleic acid molecule (e.g., RNA molecule) comprising an RdRP-responsive 5′ UTR (e.g., a flavivirus 5′ UTR) and a second exogenous nucleic acid sequence (e.g., a gene of interest, e.g., encoding an exogenous protein, e.g., a therapeutic protein);

wherein the first nucleic acid molecule and the second nucleic acid molecule are each non-naturally occurring nucleic acid molecules. In some embodiments, the first exogenous nucleic acid sequence and the second exogenous nucleic acid sequence each comprise a different gene of interest (e.g., encoding different exogenous proteins and/or different therapeutic agents). In some embodiments, the composition further comprises a third nucleic acid molecule comprising a nucleic acid sequence encoding an RdRP and/or a helicase. In some embodiments, the composition is inside a cell. e.g., an erythroid cell, e.g., an enucleated erythroid cell.

The present disclosure also provides, in some aspects, a cell. e.g., an erythroid cell, e.g., a nucleated erythroid cell precursor or an enucleated erythroid cell, comprising a nucleic acid construct as described herein. In some embodiments, the cell is a mammalian cell (e.g., a human cell. e.g., a human blood cell. e.g., a human erythroid cell).

The present disclosure also provides, in some aspects, a cell, e.g., an erythroid cell, e.g., a nucleated erythroid cell precursor or an enucleated erythroid cell, comprising a composition as described herein. In some embodiments, the cell further comprises an RNA molecule comprising an RdRP-responsive 5′ UTR and/or an RdRP-responsive 3′ UTR, wherein the RNA molecule does not comprise an exogenous nucleic acid sequence encoding an exogenous protein.

The present disclosure also provides, in some aspects, a method of making a cell comprising an exogenous therapeutic agent, the method comprising:

(i) introducing the nucleic acid construct (e.g., RNA construct) of any one of claims 18-37 or a composition of any of claims 39-58 into a cell, and

(ii) culturing the cell under conditions whereby the exogenous nucleic acid sequence is expressed to produce an exogenous therapeutic agent;

thereby making a cell comprising a therapeutic agent.

The present disclosure also provides, in some aspects, a method of making a cell comprising an exogenous therapeutic agent, the method comprising:

(i) introducing the nucleic acid construct (e.g., RNA construct) of any one of claims 18-37 or a composition of any of claims 39-58 into a cell, and

(ii) culturing the cell under conditions whereby the non-viral sequence is expressed to produce an exogenous therapeutic agent;

thereby making a cell comprising a therapeutic agent.

The present disclosure also provides, in some aspects, a method of making an erythroid cell comprising an exogenous therapeutic agent, the method comprising:

(i) introducing a nucleic acid construct (e.g., RNA construct) as described herein or a composition as described herein into a nucleated erythroid cell precursor, and

(ii) culturing the nucleated erythroid cell precursor under conditions whereby the exogenous nucleic acid sequence is expressed to produce an exogenous therapeutic agent, optionally wherein the culturing results in enucleation of the nucleated erythroid cell precursor;

thereby making an erythroid cell comprising a therapeutic agent.

The present disclosure also provides, in some aspects, a method of making an erythroid cell comprising an exogenous therapeutic agent, the method comprising:

(i) introducing a nucleic acid construct (e.g., RNA construct) as described herein or a composition as described herein into a nucleated erythroid cell precursor, and

(ii) culturing the nucleated erythroid cell precursor under conditions whereby the non-viral sequence is expressed to produce an exogenous therapeutic agent, optionally wherein the culturing results in enucleation of the nucleated erythroid cell precursor;

thereby making an erythroid cell comprising a therapeutic agent.

The present disclosure also provides, in some aspects, a method of inducing delayed expression of an exogenous therapeutic agent in a cell (e.g., an erythroid cell), the method comprising:

(i) introducing into a cell a negative strand construct comprising an RdRP-dependent 5′ UTR operably linked to a nucleic acid sequence encoding the exogenous therapeutic agent.

(ii) introducing a compatible RdRP into the cell (e.g., by introducing an RdRP or a nucleic acid encoding the RdRP, e.g., before (i), after (i), or simultaneously with (i); and

(iii) culturing the cell under conditions whereby the exogenous therapeutic agent is expressed, e.g., under conditions wherein a positive strand construct is produced from the negative strand construct, and wherein the positive strand construct is translated to produce the exogenous therapeutic agent;

thereby inducing delayed expression of the exogenous therapeutic agent.

The present disclosure also provides, in some aspects, a method of administering an enucleated erythroid cell to a subject, e.g., a human subject, comprising administering to the subject an enucleated erythroid cell made according to a method described herein, thereby administering the enucleated erythroid cell to the subject.

The present disclosure also provides, in some aspects, a method of delivering an exogenous protein to a subject, e.g., a human subject, comprising administering to the subject an enucleated erythroid cell made according to a method described herein, which comprises the exogenous protein, thereby delivering the exogenous protein to the subject.

The present disclosure also provides, in some aspects, a nucleic acid construct (e.g., DNA or RNA construct) comprising an RdRP-responsive 5′ UTR (e.g., an alphavirus 5′ UTR or flavivirus 5′ UTR) and an exogenous nucleic acid sequence comprising an exogenous gene, wherein the nucleic acid construct does not comprise any sequences encoding viral structural proteins. The present disclosure further provides, in some aspect, an erythroid cell, e.g., a nucleated erythroid cell precursor or an enucleated erythroid cell, comprising the nucleic acid construct.

The present disclosure also provides, in some aspects, a nucleic acid construct according to any of FIGS. 1A-1U, or any combination thereof.

The present disclosure also provides, in some aspects, a DNA construct encoding any of the RNA constructs described herein. The present disclosure also provides, in some aspects, methods of expressing a gene of interest comprising introduce the DNA construct to a cell, whereby the cell transcribes the DNA construct to produce the RNA construct of interest.

Any of the aspects herein, e.g., the nucleic acid constructs, compositions, cells (e.g., erythroid cells), and methods above, can be combined with one or more of the embodiments herein, e.g., an embodiment below.

In some embodiments, the nucleic acid molecule does not comprise a nucleic acid sequence encoding one or more functional flavivirus structural proteins, e.g., flavivirus C, prM, and E proteins or any combination thereof. In some embodiments, the flavivirus structural protein is prM protein. In some embodiments, the nucleic acid molecule does not comprise a nucleic acid sequence encoding the flavivirus prM protein. In some embodiments, the nucleic acid molecule does not comprise any nucleic acid sequences encoding flavivirus structural proteins, e.g., flavivirus C, prM, and E proteins. In some embodiments, the nucleic acid molecule does not comprise any nucleic acid sequences encoding flavivirus structural proteins chosen from flavivirus C and E proteins.

In some embodiments, the nucleic acid molecule does not encode a functional flavivirus nonstructural protein chosen from NS1, NS2A, NS2B, NS3, NS4A, NS4B, or NS5, or any combination thereof. In some embodiments, the nucleic acid molecule does not encode a flavivirus nonstructural protein selected from NS1, NS2A, NS2B, NS3, NS4A, NS4B, and NS5, or any combination thereof. In some embodiments, the nucleic acid molecule does not encode a flavivirus nonstructural protein chosen from NS1, NS2A, NS2B, NS3, NS4A, NS4B, or NS5, or any combination thereof. In some embodiments, the nucleic acid molecule does not comprise a nucleic acid sequence encoding a functional NS1 protein. In some embodiments, the nucleic acid molecule does not comprise a nucleic acid sequence encoding an NS1 protein.

In some embodiments, the nucleic acid molecule does not comprise any sequences encoding flavivirus nonstructural genes, e.g., genes encoding flavivirus NS1, NS2A, NS2B, NS3, NS4A, NS4B, and/or NS5 proteins. In some embodiments, the nucleic acid molecule further comprises one or more flavivirus nonstructural proteins, e.g., NS1, NS2A, NS2B, NS3, NS4A, NS4B, or NS5, or any combination thereof. In some embodiments, the nucleic acid molecule comprises a nucleic acid sequence encoding one or more flavivirus nonstructural proteins, e.g., NS1, NS2A, NS2B, NS3, NS4A, NS4B, or NS5, or any combination thereof. In certain embodiments, the one or more flavivirus nonstructural proteins comprises an RdRP and/or a helicase.

In some embodiments, the cell further comprises a second RNA molecule encoding a functional RNA-dependent RNA polymerase (RdRP).

In some embodiments, the cell further comprises a second exogenous nucleic acid molecule encoding a functional RNA-dependent RNA polymerase (RdRP). In certain embodiments, the cell further comprises a third exogenous nucleic acid encoding a nuclease and/or a helicase.

In some embodiments, the cell further comprises a functional RNA-dependent RNA polymerase (RdRP).

In some embodiments, the cell further comprises an exogenous non-flaviviral protein or noncoding RNA encoded by the non-flaviviral gene.

In some embodiments, the cell comprises a non-flaviviral protein. In some embodiments, the cell further comprises helicase and/or RdRP. In some embodiments, the cell does not comprise helicase and/or RdRP.

In some embodiments, the non-flaviviral gene encodes a therapeutic agent.

In some embodiments, the non-flaviviral gene encodes an antibody molecule, enzyme, cytokine, cytokine receptor, chemokine, chemokine receptor, interleukin, receptor, ligand, hormone, growth factor, blood factor, lysosomal storage enzyme, immune stimulatory molecule (e.g., a costimulatory molecule), intein, DNA-binding protein, RNA-binding protein, a complement regulatory molecule, a complement cascade molecule, a clotting cascade molecule, a chelator, or a functional fragment thereof.

In some embodiments, the nucleic acid further comprises an RdRP-responsive 3′ UTR. In some embodiments, the nucleic acid further comprises a 3′ flavivirus UTR.

In some embodiments, the cell does not comprise flavivirus DNA, e.g., does not comprise full length flavivirus DNA or does not comprise an RdRP-responsive 5′ UTR (e.g., a flavivirus 5′ UTR), a flavivirus 3′ UTR), a flavivirus structural gene, or a flavivirus non-structural gene. In some embodiments, the cell does not comprise viral DNA, e.g., does not comprise full length viral DNA or does not comprise an RdRP-responsive 5′ UTR, an RdRP-responsive 3′ UTR, a viral structural gene, or a viral non-structural gene.

In some embodiments, the nucleic acid construct lacks at least one gene encoding a functional flavivirus nonstructural protein, e.g., flavivirus NS1, NS2A, NS2B, NS3, NS4A, NS4B, or NS5, or any combination thereof. In some embodiments, the nucleic acid construct lacks at least one gene encoding a flavivirus nonstructural protein, e.g., flavivirus NS1, NS2A, NS2B, NS3, NS4A, NS4B, or NS5, or any combination thereof. In some embodiments, the nucleic acid construct lacks a gene encoding a functional flavivirus NS1 protein. In some embodiments, the nucleic acid construct lacks a gene encoding a flavivirus NS1 protein.

In some embodiments, the nucleic acid construct lacks at least a portion of a gene encoding a flavivirus structural protein. In some embodiments, the nucleic acid construct comprises a fragment of a gene encoding a flaviviral structural protein. In some embodiments, the nucleic acid construct lacks at least one gene encoding a functional flavivirus structural protein, e.g., flavivirus C, prM, or E protein, or any combination thereof. In some embodiments, the nucleic acid construct lacks at least one gene encoding a flavivirus structural protein, e.g., flavivirus C, prM, or E protein, or any combination thereof.

In some embodiments, the exogenous gene comprises a gene encoding a fusion protein that comprises a first domain (e.g., a non-flaviviral protein, or a portion thereof) and a transmembrane domain, e.g., a Type I, Type II, or Type III transmembrane domain. In certain embodiments, the transmembrane domain comprises a transmembrane domain present endogenously in mammalian, e.g., human, erythroid cells, e.g., erythrocytes.

In some embodiments, the nucleic acid construct does not comprise a 3′ UTR (e.g., an RdRP-responsive 3′ UTR). In certain embodiments, the nucleic acid construct does not comprise a flavivirus 3′ UTR.

In some embodiments, the nucleic acid construct further comprises a 3′ UTR, e.g., an RdRP-responsive 3′ UTR. In certain embodiments, the nucleic acid construct comprises a flavivirus 3′ UTR.

In some embodiments, the flavivirus is Dengue virus. In certain embodiments, the flavivirus is a Dengue virus serotype selected from the group consisting of DEN-1. DEN-2, DEN-3, and DEN-4.

In some embodiments, nucleic acid construct is an RNA construct. In certain embodiments, the RNA construct comprises a 5′ cap, a poly-A tail, or both of a 5′ cap and a poly-A tail. In certain embodiments, the RNA construct comprises a poly-A tail and does not comprise a flaviviral 3′ UTR. In certain embodiments, the RNA construct comprises a flaviviral 3′ UTR and does not comprise a poly-A tail.

In some embodiments, the RdRP encoded by the second nucleic acid molecule is a flavivirus RdRP. In some embodiments, the RdRP encoded by the second nucleic acid molecule is a Dengue virus RdRP, e.g., Dengue virus NS5 protein.

In some embodiments, the RdRP-responsive 5′ UTR is a flavivirus 5′ UTR. In certain embodiments, the flavivirus 5′ UTR is a Dengue virus 5′ UTR. e.g., a 5′UTR from a Dengue virus serotype chosen from DEN-1, DEN-2, DEN-3, or DEN-4.

In some embodiments, the first nucleic acid molecule does not comprise any sequences encoding one or more functional flavivirus structural proteins, e.g., flavivirus C, prM, and E proteins. In some embodiments, the first nucleic acid molecule does not comprise any sequences encoding one or more functional flavivirus nonstructural genes, e.g., genes encoding flavivirus NS1, NS2A, NS2B, NS3, NS4A, NS4B, and NS5 proteins. In certain embodiments, the first nucleic acid molecule does not comprise any sequences encoding a functional NS1 protein. In certain embodiments, the second nucleic acid molecule does not comprise any sequences encoding a functional NS1 protein. In some embodiments, the rust nucleic acid molecule further comprises a flavivirus 3′ UTR. In some embodiments, the second nucleic acid molecule does not comprise a flavivirus 3′ UTR or a flavivirus 5′ UTR. In other embodiments, the second nucleic acid molecule comprises a flavivirus 3′ UTR and a flavivirus 5′ UTR.

In some embodiments, the second nucleic acid construct does not comprise any sequences encoding one or more functional flavivirus structural proteins. e.g., flavivirus C, prM, and E proteins. In some embodiments, the second nucleic acid construct does not comprise any sequences encoding a flavivirus structural protein, e.g., flavivirus C, prM, and E proteins.

In some embodiments, the exogenous (e.g., non-viral) sequence comprises a bacterial, archaean, or eukaryotic sequence. In certain embodiments, the exogenous nucleic acid sequence is a mammalian, plant, fungal, or insect sequence. In certain embodiments, the exogenous nucleic acid sequence is a human sequence.

In some embodiments, the exogenous nucleic acid sequence encodes a polypeptide, e.g., a protein. In certain embodiments, the exogenous nucleic acid sequence encodes an antibody molecule, or an antigen-binding fragment thereof, e.g., an scFv, or a T cell receptor, or an antigen-binding fragment thereof. In certain embodiments, the exogenous nucleic acid sequence encodes an antibody, or an antigen-binding fragment thereof, e.g., an scFv, or a T cell receptor, or an antigen-binding fragment thereof. In certain embodiments, the exogenous nucleic acid sequence encodes a fusion protein comprising a first domain (e.g., a non-viral protein, or a portion thereof) and a transmembrane domain, e.g., a Type I. Type II, or Type III transmembrane domain. In certain embodiments, the fusion protein comprises an antibody molecule, or an antigen-binding fragment thereof, e.g., an scFv. In certain embodiments, the fusion protein comprises a heavy chain, a light chain, or a heavy chain and a light chain. In certain embodiments, the fusion protein is an antibody, or an antigen-binding fragment thereof, e.g., an scFv. In certain embodiments, the transmembrane domain comprises a transmembrane domain present endogenously in mammalian, e.g., human, erythroid cells, e.g., erythrocytes.

In some embodiments, the exogenous nucleic acid sequence encodes a noncoding RNA. e.g., a microRNA, ribozyme, aptamer, siRNA, piRNA, snoRNA, snRNA, long ncRNA, or tRNA.

In some embodiments, the first nucleic acid molecule does not comprise a 3′ UTR (e.g., an RdRP-responsive 3′ UTR, e.g., a flavivirus 3′ UTR) and/or the second nucleic acid molecule does not comprise a 3′ UTR (e.g., an RdRP-responsive 3′ UTR. e.g., a flavivirus 3′ UTR).

In some embodiments, the first nucleic acid molecule further comprises a 3′ UTR (e.g., an RdRP-responsive 3′ UTR, e.g., a flavivirus 3′ UTR), and/or the second nucleic acid molecule further comprises a 3′ UTR (e.g., an RdRP-responsive 3′ UTR, e.g., a flavivirus 3′ UTR).

In some embodiments of any of the methods herein, the cell is a mammalian cell (e.g., a human cell), a plant cell, bacterial cell, fungal cell, or insect cell.

In some embodiments of any of the methods herein, introducing the nucleic acid construct or composition into the cell comprises using electroporation or transfection, e.g., LNP-mediated transfection.

In some embodiments, the nucleic acid construct encodes a fragment of a viral structural protein, e.g., a flavivirus protein C, e.g., a 5′ fragment of about 100 nucleotides or about 90-110, 80-120, 70-130, 60-140, or 50-150 nucleotides, e.g., situated between the RdRP-responsive 5′ UTR and a coding region (e.g., a coding region for an exogenous protein). In some embodiments, the nucleic acid comprises one or more (e.g., two) cyclization sequence (CS) regions (e.g., a 5′ CS or 3′ CS region as described in Gebhard et al. 2011; Viruses 3: 1739-1756). In some embodiments, a CS region is adjacent to the 5′ UTR. In embodiments, a CS region is a cis-acting element (e.g., of 11 nucleotides) found within the N-terminal 34 amino acids of the C protein and promotes interaction of the 3′UTR with the 5′ UTR. In some embodiments, the cis-acting element is situated such that it is translated, and in other embodiments, the cis-acting element is situated such that it is not translated.

In some embodiments, the nucleic acid encodes a fragment of a viral structural protein, e.g., a flavivirus protein E, e.g., a 3′ fragment of about 93 nucleotides, e.g., about 80-100 or 60-120 nucleotides. In embodiments, the fragment of flavivirus protein E is situated adjacent to the gene for NS1. In embodiments, the fragment of flavivirus protein E promotes correct topology of NS1.

In some embodiments, the nucleic acid comprises an IRES, e.g., situated between the RdRP-responsive 5′ UTR and a coding region (e.g., a coding region for an exogenous protein or an RdRP). In some embodiments, the IRES is situated between the RdRP-responsive 3′ UTR and a coding region (e.g., a coding region for an exogenous protein or an RdRP). In some embodiments, the IRES is situated upstream (e.g., immediately upstream) of the coding region (e.g., a coding region for an exogenous protein or an RdRP).

In some embodiments, the nucleic acid comprises a protease cleavage site, e.g., an FMDV2A cleavage site or a flavivirus structural protein E cleavage site, e.g., situated between a coding region for an exogenous protein and a coding region for an RdRP.

In some embodiments, the nucleic acid lacks nonstructural protein NS1. In some embodiments, the nucleic acid lacks nonstructural protein NS2A. In some embodiments, the nucleic acid lacks nonstructural protein NS2B. In some embodiments, the nucleic acid lacks nonstructural protein NS3. In some embodiments, the nucleic acid lacks nonstructural protein NS4A. In some embodiments, the nucleic acid lacks nonstructural protein NS4B. In some embodiments, the nucleic acid lacks nonstructural protein NS5.

In some embodiments, the nucleic acid comprises RdRP and one or more protein cofactors. In some embodiments, the nucleic acid comprises nonstructural proteins NS3 and NS5. In some embodiments, the nucleic acid comprises nonstructural proteins NS1 and NS5. e.g., NS1, NS3, and NS5. In some embodiments, the nucleic acid comprises NS5 and one or more of (e.g., 2, 3, 4, 5, or all of) NS1, NS2A, NS2B, NS3, NS4A, NS4B.

In some embodiments, the nucleic acid has a length of 1-2 kb, 1-3 kb, 1-4 kb, 1-5 kb, 5-10, or 10-20 kb.

In some embodiments, the 5′ UTR (e.g., on a positive strand) comprises a sequence of SEQ ID NO: 5, or an RdRP-responsive sequence having at least 80%, 85%, 90%, 95%, 96%, 97%, 98%, or 99% identity thereto. In some embodiments, the 3′ UTR (e.g., on a positive strand) comprises a sequence of SEQ ID NO: 6, or an RdRP-responsive sequence having at least 80%, 85%, 90%, 95%, 96%, 97%, 98%, or 99% identity thereto. In some embodiments, the 5′ UTR (e.g., on a negative strand) comprises a sequence of SEQ ID NO: 7, or an RdRP-responsive sequence having at least 80%, 85%, 90%, 95%, 96%, 97%, 98%, or 99% identity thereto. In some embodiments, the 3′ UTR (e.g., on a negative strand) comprises a sequence of SEQ ID NO: 8, or an RdRP-responsive sequence having at least 80%, 85%, 90%, 95%, 96%, 97%, 98%, or 99% identity thereto. In some embodiments, a variant of a 3′ UTR or a 5′ UTR has the same secondary structure as any of SEQ ID NOS: 5-8, e.g., is predicted to have double stranded regions at the same location, e.g., comprises one or more pairs of compensatory mutations in double stranded regions. In some embodiments, the RdRP comprises a sequence of SEQ ID NO: 9, or an enzymatically active sequence having at least 80%, 85%, 90%, 95%, 96%, 97%, 98%, or 99% identity thereto.

The disclosure contemplates all combinations of any one or more of the foregoing aspects and/or embodiments, as well as combinations with any one or more of the embodiments set forth in the detailed description and examples.

All publications, patent applications, patents, and other references (e.g., sequence database reference numbers) mentioned herein are incorporated by reference in their entirety.

For example, all GenBank, Unigene, and Entrez sequences referred to herein, e.g., in any Table herein, are incorporated by reference. Unless otherwise specified, any sequence accession numbers specified herein, including in any Table herein, refer to the database entries current as of Jan. 10, 2018. When one gene or protein references a plurality of sequence accession numbers, all of the sequence variants are encompassed.

### BRIEF DESCRIPTION OF THE FIGURES

FIGS. 1A, 1B, 1C, 1D, 1E, 1F, 1G, 1H, 1I, 1J, 1K, 1L, 1M, 1N, 1O, 1P, 1Q, 1R, 1S, 1T, and 1U are a series of diagrams showing possible orientations of elements within nucleic acid constructs of the present disclosure. FIGS. 1A-1E show constructs comprising one or more of an RdRP-responsive 5′ UTR (e.g., a flavivirus 5′ UTR), an exogenous nucleic acid sequence (e.g., comprising a gene of interest, e.g., encoding an exogenous polypeptide), and/or a flavivirus 3′ UTR. FIGS. 1F-1M show constructs comprising one or more of a flavivirus 5′ UTR, a sequence encoding a flavivirus NS5 RNA-dependent RNA polymerase (RdRP), and/or a flavivirus 3′ UTR. FIGS. 1N-1U show constructs comprising one or more of a flavivirus 5′ UTR, an exogenous nucleic acid sequence, a sequence encoding a flavivirus RdRP (e.g., an RdRP compatible with the flavivirus 5′ UTR), and/or a flavivirus 3′ UTR.

FIGS. 2A and 2B are diagrams showing replication of exemplary nucleic acid constructs of the present disclosure. In FIG. 2A, a positive strand RNA construct comprising, in order from 5′ to 3′, a flavivirus 5′ UTR, an exogenous nucleic acid sequence, and a flavivirus 3′ UTR, binds to a compatible RdRP, which proceeds to synthesize the reverse complement strand, a negative strand RNA. Because this negative strand RNA also comprises both 5′ and 3′ UTR sequences, the RdRP can also bind to it and direct synthesis of a strand matching the sequence of the original positive strand RNA, thereby leading to exponential amplification of both positive and negative strand RNA constructs. In FIG. 2B, a negative strand RNA construct comprising, in order from 3′ to 5′, a flavivirus 5′ UTR and an exogenous nucleic acid sequence, binds to a compatible RdRP, which proceeds to synthesize the reverse complement strand, a positive strand RNA. The constructs may also include an internal ribosomal entry site (IRES), e.g., to help drive translation of the positive strand.

FIGS. 3A and 3B show expression of a nucleic acid construct in erythroid cells. FIG. 3A depicts the dengue replicon used in the experiment. FIG. 3B is a graph showing the mean fluorescence intensity (MFI) measured after transduction of erythroid cells at day 4 of maturation phase with constructs encoding GFP. RNA 0.5 ug/20 uL replicon=RNA replicon comprising, in order from 5′ to 3′, a Dengue virus 5′ UTR, a sequence encoding a nonfunctional 34 amino acid fragment of a Dengue virus core protein (C), a gene encoding GFP, an FMDV2A cleavage site, an internal ribosomal entry site (IRES), a sequence encoding a nonfunctional 24-amino acid fragment of a Dengue virus envelope (E) protein, a sequence encoding Dengue virus nonstructural proteins, and a Dengue virus 3′ UTR. DNA 5 ug/20 uL replicon=a circular DNA plasmid comprising the sequence of the above RNA replicon. GFP mRNA=mRNA molecules encoding GFP and capable of being translated to produce GFP protein. GFP plasmid=an expression plasmid comprising a sequence encoding GFP, from which GFP mRNA can be transcribed and GFP protein can then be produced.

FIGS. 4A and 4B show several RNA constructs tested for their ability to direct the translation of protein. FIG. 4A. Schematics of five RNA constructs each including a particular arrangement of RdRP-responsive 5′ UTR, IRES. GFP-encoding sequence. RdRP-responsive 3′ UTR, and/or poly-A tail. The first RNA construct (“GFP control”) consists of, from 5′ to 3′, a 5′ alpha globin UTR, a eGFP-encoding sequence (“GFP”), and a 3′ alpha globin UTR. The second construct (“5A-GFP-3A”) consists of, from 5′ to 3′, a Dengue virus Type II-derived RdRP-responsive 5′ UTR (“5-AMP”), a eGFP-encoding sequence, and Dengue virus Type II-derived RdRP-responsive 3′ UTR. (“3-AMP”). The third construct (“5A-GFP-3A-PolyA”) consists of, from 5′ to 3′, a Dengue virus Type II-derived RdRP-responsive 5′ UTR, a eGFP-encoding sequence, a Dengue virus Type II-derived RdRP-responsive 3′ UTR, and a 120 nt-poly-A tail. The fourth construct (“5A-IRES-TRILINK UTRs GFP-3A”) consists of, from 5′ to 3′, a Dengue virus Type II-derived RdRP-responsive 5′ UTR, an MSCV IRES, a 5′ alpha globin UTR, eGFP-encoding sequence, a 3′ alpha globin UTR, and a Dengue virus Type II-derived RdRP-responsive 3′ UTR. The fifth construct (“5A-IRES-GFP-3A-PolyA”) consists of, from 5′ to 3′, a Dengue virus Type II-derived RdRP-responsive 5′ UTR, IRES. GFP-encoding sequence, a Dengue virus Type II-derived RdRP-responsive 3′ UTR, and a 120 nt poly-A tail. Each of the RNA constructs also a 5′ Cap (CleanCap Cap1 (TRILINK)). FIG. 4B GFP expression detected in each of the RNA constructs.

FIG. 5 is a schematic showing an RNA amplification method in which a positive strand RNA construct (RNA 1) comprising an RdRP-responsive 5′ UTR, an RNA sequence encoding a gene of interest, and an RdRP-responsive 3′ UTR, is introduced into a cell. Also introduced into the cell (e.g., concurrently or separately) is a RNA construct (RNA 2) comprising an RdRP-responsive 5′ UTR, an RNA sequence encoding an RdRP (e.g., a Dengue NS5 protein, as shown), and an RdRP-responsive 3′ UTR. In this approach, the positive strand RNA construct (i.e., RNA 1) can be translated to produce the protein encoded by the gene immediately upon introduction into the cell. In addition, the positive strand RNA construct can interact with and be subject to replication by the RdRP (e.g., the RdRP encoded by RNA 2) to produce negative strand copies. These negative strand copies can then interact with and be replicated by the RdRP to produce additional positive strand copies, which can then be subjected to translation and/or further replication.

FIG. 6 is a schematic showing an RNA amplification method in which a negative strand RNA construct (RNA 1) comprising a RdRP-responsive 3′ UTR, an RNA sequence encoding a gene of interest, and a RdRP-responsive 5′ UTR, is introduced into a cell. Also introduced into the cell (e.g., concurrently or separately) is a second RNA construct (RNA 2) comprising an RdRP-responsive 5′ UTR, an RNA sequence encoding an RdRP (e.g., a Dengue NS5 protein, as shown), and an RdRP-responsive 3′ UTR. In this approach, the negative strand RNA construct can interact with the RdRP (e.g., encoded by RNA 2) to produce positive strand copies, which can in turn be translated to produce protein. In addition, the positive strands can interact with and be replicated by the RdRP to produce additional negative strands copies (which can in turn be used to produce additional positive strands). In some embodiments, the introduction of negative strand RNA constructs to a cell delays the production of exogenous protein, as the RNA construct must first be processed by the RdRP in order to produce a suitable template for the cellular translation machinery.

### DETAILED DESCRIPTION OF THE INVENTION

### Definitions

As used herein, the term “antibody molecule” refers to a protein, e.g., an immunoglobulin chain or fragment thereof, comprising at least one immunoglobulin variable domain sequence. In some embodiments, the antibody molecule binds specifically to a target, such as a carbohydrate, polynucleotide, polypeptide, etc., through at least one antigen recognition site, located in the variable region of the immunoglobulin molecule. The term “antibody molecule” encompasses antibodies and antibody fragments (e.g., Fab, Fab′, F(ab′)2, Fv), single chain (ScFv) and domain antibodies), and fusion proteins including an antibody portion, and any other modified configuration of an immunoglobulin molecule that includes an antigen recognition site.

In an embodiment, an antibody molecule is a multispecific antibody molecule, e.g., a bispecific antibody molecule. Examples of antibody molecules include, but are not limited to, Fab, Fab′, F(ab′)2, Fv fragments, scFv antibody fragments, disulfide-linked Fvs (sdFv), a Fd fragment consisting of the VH and CH1 domains, linear antibodies, single domain antibodies such as sdAb (either VL or VH), camelid VHH domains, multi-specific antibodies formed from antibody fragments such as a bivalent fragment comprising two Fab fragments linked by a disulfide bridge at the hinge region, an isolated epitope binding fragment of an antibody, maxibodies, minibodies, nanobodies, intrabodies, diabodies, triabodies, tetrabodies, v-NAR and bis-scFv.

A “variable region” of an antibody molecule refers to the variable region of the antibody molecule light chain or the variable region of the antibody molecule heavy chain, either alone or in combination. As known in the art, the variable regions of the heavy and light chain each consist of four framework regions (FR) connected by three complementarity determining regions (CDRs) that contain hypervariable regions. The CDRs in each chain are held together in close proximity by the FRs and, with the CDRs from the other chain, contribute to the formation of the antigen-binding site of antibodies. The positions of the CDRs and FRs may be determined using various well-known methods, e.g., Kabat, Chothia, the international ImMunoGeneTics database (IMGT) (on the worldwide web at imgt.org), and AbM (see, e.g., Johnson et al. Nucleic Acids Res., 29:205-206 (2001); Chothia and Lesk, J. Mol. Biol., 196:901-917 (1987); Chothia et al, Nature, 342:877-883 (1989); Chothia et al, J. Mol. Biol., 227:799-817 (1992); Al-Lazikani et al, J. Mol. Biol, 273:927-748 (1997)). In some embodiments, the CDRs of an antibody molecule are determined according to Kabat, Chothia, or a combination thereof.

In some embodiments, the antibody molecule is a monoclonal antibody molecule. As used herein. “monoclonal antibody molecule” or “monoclonal antibody” refers to an antibody molecule obtained from a population of substantially homogeneous antibody molecules. e.g., wherein individual antibodies including the population are identical except for possible naturally-occurring mutations that may be present in minor amounts. Monoclonal antibodies are highly specific, being directed against a single antigenic site. Furthermore, in contrast to polyclonal antibody preparations, which typically include different antibodies directed against different determinants (epitopes), each monoclonal antibody is directed against a single determinant on the antigen.

“Derived from” as that term is used herein, indicates a relationship between a first and a second molecule. It generally refers to structural similarity between the first molecule and a second molecule and does not connote or include a process or source limitation on a first molecule that is derived from a second molecule.

As used herein, “enucleated” refers to a cell. e.g., a reticulocyte or mature red blood cell, that lacks a nucleus. In an embodiment an enucleated cell is a cell that has lost its nucleus through differentiation from a precursor cell, e.g., a hematopoietic stem cell (e.g., a CD34+ cell), a common myeloid progenitor (CMP), a megakaryocyte erythrocyte progenitor cell (MEP), a burst-forming unit erythrocyte (BFU-E), a colony-forming unit erythrocyte (CFU-E), a pro-erythroblast, an early basophilic erythroblast, a late basophilic erythroblast, a polychromatic erythroblast, or an orthochromatic erythroblast, or an induced pluripotent cell, into a reticulocyte or mature red blood cell. In an embodiment an enucleated cell is a cell that has lost its nucleus through in vitro differentiation from a precursor cell, e.g., a hematopoietic stem cell (e.g., a CD34+ cell), a common myeloid progenitor (CMP), a megakaryocyte erythrocyte progenitor cell (MEP), a burst-forming unit erythrocyte (BFU-E), a colony-forming unit erythrocyte (CFU-E), a pro-erythroblast, an early basophilic erythroblast, a late basophilic erythroblast, a polychromatic erythroblast, or an orthochromatic erythroblast, or an induced pluripotent cell into a reticulocyte or mature red blood cell.

“Erythroid cell” as used herein, includes a nucleated red blood cell, a red blood cell precursor (e.g., an erythroid cell precursor), an enucleated mature red blood cell, and a reticulocyte. For example, any of a cord blood stem cell, a CD34+ cell, a hematopoietic stem cell (HSC), a spleen colony forming (CFU-S) cell, a common myeloid progenitor (CMP) cell, a blastocyte colony-forming cell, a burst forming unit-erythroid (BFU-E), a megakaryocyte-erythroid progenitor (MEP) cell, an erythroid colony-forming unit (CFU-E), a reticulocyte, an erythrocyte, an induced pluripotent stem cell (iPSC), a mesenchymal stem cell (MSC), a polychromatic normoblast, an orthochromatic normoblast, is an erythroid cell. A preparation of erythroid cells can include any of these cells or a combination thereof. In some embodiments, the erythroid cells are immortal or immortalized cells. For example, immortalized erythroblast cells can be generated by retroviral transduction of CD34+ hematopoietic progenitor cells to express Oct4, Sox2, Klf4, cMyc, and suppress TP53 (e.g., as described in Huang et al. (2014) Mol. Ther. 22(2): 451-463). In addition, the cells may be intended for autologous use or provide a source for allogeneic transfusion. In some embodiments, erythroid cells are cultured. In an embodiment an erythroid cell is an enucleated red blood cell. In some instances, the term “erythroid cell” refers to a red blood cell (RBC).

As used herein, the term “exogenous nucleic acid” is used to refer to a nucleic acid that is not present in a wild-type cell or virus of a given type or is present at a lower level in a wild-type cell or virus than in a cell or virus containing the exogenous nucleic acid. In some embodiments, an exogenous nucleic acid is a nucleic acid, e.g., an RNA, that was introduced into the cell. In some embodiments, an exogenous nucleic acid is a nucleic acid, e.g., an RNA, that was produced in the cell, e.g., transcribed from an exogenous DNA sequence. In embodiments, the exogenous nucleic acid is an RNA or a DNA. In some embodiments, the exogenous nucleic acid is exogenous to the virus from which the 5′ UTR is derived; the exogenous nucleic acid may, in embodiments, also be exogenous to an erythroid cell in which the exogenous nucleic acid is disposed. In some embodiments, “exogenous nucleic acid” refers to a portion of a nucleic acid construct (e.g., as described herein). In certain embodiments, this portion comprises a nucleic acid sequence encoding at least one exogenous protein (e.g., a protein exogenous to one or more flavivirus-derived nucleic acid sequences in the nucleic acid construct and/or a protein exogenous to a cell into which the nucleic acid construct is introduced). In certain embodiments, the exogenous protein comprises a therapeutic agent. In some embodiments, the exogenous nucleic acid comprises a nucleic acid encoding an RdRP (e.g., NS5). In some embodiments, the exogenous nucleic acid comprises a nucleic acid encoding a helicase (e.g., NS3). In some embodiments, the exogenous nucleic acid comprises a nucleic acid encoding an exogenous protein and an RdRP. In some embodiments, the exogenous nucleic acid comprises a nucleic acid encoding an exogenous protein, an RdRP, and a helicase.

As used herein, the terms “exogenous polypeptide” and “exogenous protein” with respect to a cell are used interchangeably to refer to a polypeptide that is not produced by a wild-type cell of a given type or is present at a lower level in a wild-type cell than in a cell containing the exogenous polypeptide. In some embodiments, an exogenous polypeptide is a polypeptide encoded by a nucleic acid that was introduced into the cell, which nucleic acid is optionally not retained by the cell. In some embodiments, an exogenous polypeptide is a polypeptide conjugated to the surface of the cell by chemical or enzymatic means.

As used herein, the terms “flavivirus 5′ UTR” refer to an RdRP-responsive 5′ UTR derived from flavivirus. In some embodiments, the flavivirus 5′ UTR is a wild-type sequence or a reverse complement thereof. In other embodiments, the flavivirus 5′ UTR is an active mutant or variant of a reverse complement of the active mutant.

As used herein, the term “RdRP-responsive 5′ UTR” refers to an untranslated region of an RNA molecule that, when disposed 5′ of an RNA sequence of interest in the RNA molecule, is capable of directing synthesis of an RNA strand that is the reverse complement of the RNA molecule (or a portion thereof), in the presence of a compatible RdRP, or the reverse complement of said untranslated region. In some embodiments, an RdRP binds to the RdRP-responsive 5′ UTR. In some embodiments, e.g., on a positive strand having an RdRP-responsive 5′ UTR, a 3′ UTR is required for the RdRP to direct synthesis of a new RNA strand, e.g. by circularizing the RNA. In some embodiments, e.g., on a negative strand having an RdRP-responsive 5′ UTR (e.g., disposed at the 3′ end of the negative strand), a 3′ UTR is not required for the RdRP to direct synthesis of a new RNA strand. In some embodiments, the RdRP-responsive 5′ UTR comprises a 5′ cyclization sequence (CS) region (e.g., as described in Gebhard et al. 2011; Viruses 3: 1739-1756; incorporated herein by reference in its entirety). In some embodiments, the RdRP-responsive 5′ UTR does not comprise a 5′ CS region. In certain embodiments, the RdRP-responsive 5′ UTR is located 5′ to an open reading frame encoding an exogenous protein (e.g., comprising a therapeutic agent), wherein the open reading frame comprises a 5′ CS region. In certain embodiments, the RdRP-responsive 5′ UTR is located 5′ to an open reading frame and the 5′ CS region is located within about 30 nucleotides (e.g., within 5, 10, 11, 12, 13, 14, 15, 16, 17, 18, 19, 20, 21, 22, 23, 24, 25, or 30 nucleotides) of the 5′ end of the open reading frame. In certain embodiments, the RdRP-responsive 5′ UTR is located 5′ to an open reading frame and the 5′ CS region overlaps at least partially with, e.g., is located within, nucleotides 13-16 of the open reading frame. In some embodiments, the RdRP-responsive RdRP-responsive 5′ UTR comprises an SLA domain (e.g., comprising one, two, or all three of an S1, S2, an S3 helical region), an oligo-U spacer, and an SLB domain. e.g., as described in Gebhard et al., supra.

As used herein, the terms “flavivirus 3′ UTR” refer to an RdRP-responsive 3′ UTR derived from flavivirus. In some embodiments, the flavivirus 3′ UTR is a wild-type sequence or a reverse complement thereof. In other embodiments, the flavivirus 3′ UTR is an active mutant or variant of a reverse complement of the active mutant.

As used herein, the term “RdRP-responsive 3′ UTR” refers to an untranslated region of an RNA molecule that, when disposed at the 3′ terminal end of an RNA molecule, is capable of interacting with a compatible RdRP. In some embodiments, an RdRP binds to the RdRP-responsive 3′ UTR. In some embodiments, e.g., on a positive strand RNA having an RdRP-responsive 3′ UTR, the RdRP-responsive 3′ UTR is required for the RdRP to direct synthesis of a new RNA strand, e.g., by circularizing the RNA. In some embodiments, e.g., on a negative strand having an RdRP-responsive 3′ UTR (e.g., disposed at the 5′ end of the negative strand), the RdRP-responsive 3′ UTR is not required for the RdRP to direct synthesis of a new RNA strand. In some embodiments, the RdRP-responsive 3′ UTR comprises a 3′ CS region (e.g., as described in Gebhard et al. (2011) Viruses 3: 1739-1756). In some embodiments, the RdRP-responsive 3′ UTR comprises a Domain I, Domain II (e.g., comprising an RCS2 and/or CS2 sequence), and/or Domain III (e.g., comprising a 3′ CS region and/or a 3′SL structure). e.g., as described in Gebhard et al., supra. In certain embodiments, the RdRP-responsive 3′ UTR comprises Domain III and not Domain I or Domain II.

As used herein, the terms “peptide,” “polypeptide.” and “protein” are used interchangeably to refer to any chain of two or more natural or unnatural amino acid residues, regardless of post-translational modification (e.g., glycosylation or phosphorylation), constituting all or part of a naturally-occurring or non-naturally occurring polypeptide or peptide (e.g., a protein).

As used herein, the term “primate gene” refers to gene derived from a primate. In embodiments, the gene is wild-type. In embodiments, the gene is a fragment or variant of a wild-type gene. The gene may be codon-optimized, e.g., relative to the wild-type gene.

As used herein, the term “variant” of a polypeptide refers to a polypeptide having at least one sequence difference compared to that polypeptide, e.g., one or more substitutions, insertions, or deletions. In some embodiments, the variant has at least 70%, 80%, 85%, 90%, 95%, 96%, 97%, 98%, or 99% identity to that polypeptide. A variant includes a fragment. In some embodiments, a fragment lacks up to 1, 2, 3, 4, 5, 10, 20, or 100 amino acids on the N-terminus. C-terminus, or both (each independently), compared to the full-length polypeptide.

### Overview

The present disclosure provides amplifiable nucleic acid constructs for expression of genes of interest and methods involving same (e.g., methods for expressing the genes of interest and/or methods of amplifying the nucleic acid constructs). In one system for producing a protein (e.g., a therapeutic agent) in a cell, the RNA construct comprises an RdRP-responsive 5′ UTR (e.g., a flavivirus 5′ UTR. e.g., a Dengue virus 5′ UTR) and the cell comprises an RdRP (e.g., a flavivirus RdRP, e.g., a Dengue virus RdRP). In some instances, the flavivirus RdRP is encoded by a flavivirus NS5 gene, e.g., in the same RNA construct or in a second RNA construct.

Flavivirus RdRP generally operates by binding to a compatible 5′ UTR, such as a flavivirus 5′ UTR, and then directing synthesis of a reverse complement RNA strand. Flavivirus RdRP initiates RNA synthesis at the 3′ end of the template strand, such that synthesis of the reverse complement strand proceeds in a 5′-to-3′ direction on the new strand. Thus, for a flavivirus RdRP that binds to a 5′ UTR at the 5′ end of an RNA construct to initiate RNA synthesis, the RdRP generally also binds a 3′ UTR on the other end of the construct, leading to circularization of the RNA construct and access to its 3′ end by the RdRP. Once the RNA construct has circularized in this fashion, the flavivirus RdRP begins synthesis of the reverse complement strand. In other instances, the flavivirus RdRP can bind to a flavivirus 5′ UTR sequence located at the 3′ end of the RNA construct. In this scenario, the flavivirus RdRP can initiate RNA synthesis without necessarily binding to a 3′ UTR, as it is already positioned at the 3′ end of the template strand.

The 5′ UTR sequence need not be located at the 5′ end of the RNA construct in question. In fact, in the negative strand of the virus, the 5′ UTR sequence exists as a reverse complement of the 5′ UTR of the positive strand, and is positioned at the 3′ end of the RNA, and is competent to direct RdRP-dependent synthesis of a new RNA strand. Thus, it is contemplated that the 5′ UTR may be located at the 5′ end or the 3′ end of a nucleic acid (e.g., RNA) construct described herein. It is also contemplated that the 5′ UTR may be located 5′ to a gene of interest, but may not be located at the 5′ end of the construct. It is, alternatively, contemplated that the 5′ UTR may be located 3′ to a gene of interest, but may not be located at the 3′ end of the construct.

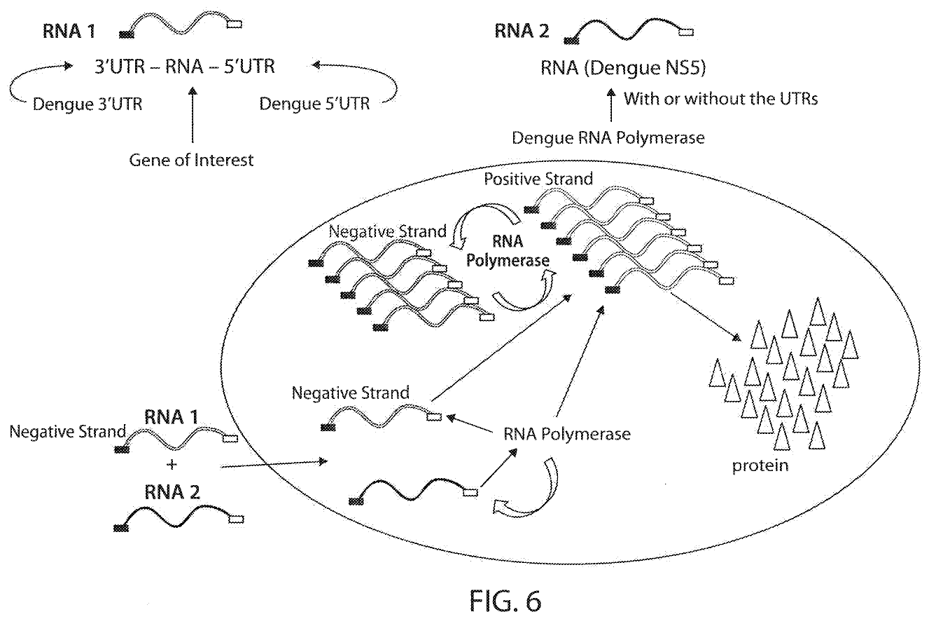

Constructs disclosed herein present numerous advantages. In some embodiments, a construct herein yields prolonged expression of an exogenous protein, e.g., compared to a construct that lacks RdRP-responsive UTRs (e.g., an RdRP-responsive 5′ UTR and/or an RdRP-responsive 3′ UTR). In some embodiments, a construct yields increased expression of an exogenous protein, e.g., compared to a construct that lacks viral UTRs. In some embodiments, the constructs described herein can be used for temporal control of expression of a gene of interest (e.g., encoding a protein, e.g., encoding a therapeutic agent) and/or amplification of the construct in a cell (e.g., by delivering negative strand RNA constructs to the cell, e.g., as shown in FIG. 6). In some embodiments, temporal control is achieved by limiting the quantity of RdRP in the cell (e.g., by introducing non-replicating RdRP-encoding nucleic acids, such that the RdRP is gradually diluted out over time, e.g., as the cell proliferates).

In some embodiments, temporal control is achieved by introducing into the cell quenching nucleic acid molecules, wherein said nucleic acid molecules comprise either an RdRP-responsive 5′ UTR sequences or an RdRP-responsive 3′ UTR sequences. These quenching nucleic acid molecules are capable of binding to an RdRP present in the cell and limit the amount of RdRP that is available for processing (e.g., amplifying) an RNA construct comprising a nucleic acid encoding an exogenous protein. The quenching nucleic acid molecules may be introduced to a cell at any given time to limit RdRP-mediated amplification of an RNA construct. In some embodiments, the quenching nucleic acid molecules (e.g., non-replicating quenching nucleic acid molecules) can be introduced to the cell alongside the nucleic acid constructs (e.g., RNA constructs) encoding the gene of interest, e.g., to delay or limit the expression of a gene encoded by the nucleic acid construct. In some embodiments, the quenching nucleic acid molecules (e.g., replicating or non-replicating quenching nucleic acid molecules) can be introduced to the cell after the nucleic acid constructs encoding the gene of interest, e.g., in order to cause expression of the gene of interest to decline over time. In some embodiments, the quenching nucleic acid molecules can be introduced to daughter cells of the cell into which the nucleic acid constructs encoding the gene of interest was introduced.

In some embodiments, a composition described herein comprises:

(i) a first nucleic acid construct comprising a 5′ UTR responsive to a first RdRP and a first exogenous nucleic acid sequence, and optionally a 3′ UTR responsive to the first RdRP;

(ii) a second nucleic acid construct comprising the first RdRP;

(iii) a third nucleic acid construct comprising a 5′ UTR responsive to a second RdRP and a second exogenous nucleic acid sequence, and optionally a 3′ UTR responsive to the second RdRP;

(iv) a fourth nucleic acid construct comprising the second RdRP. In some embodiments, the RdRP is instead provided as a protein (e.g., a protein introduced into the cell). In some embodiments, the first RdRP amplifies the first nucleic acid construct. In some embodiments, the second RdRP amplifies the third nucleic acid construct. The present disclosure also provides a cell (e.g., an erythroid cell) comprising the composition or portions thereof (e.g., one, two, three, or all four of the first, second, third, and fourth nucleic acid constructs).

### Nucleic Acid Constructs

The present disclosure provides nucleic acid constructs comprising an exogenous nucleic acid sequence encoding one or more genes of interest (e.g., a gene encoding a polypeptide or a functional RNA) that can be introduced into a cell. In some embodiments, the nucleic acid construct is an RNA construct comprising an RdRP-responsive 5′ UTR (e.g., a flavivirus 5′ UTR, e.g., as described herein) and the exogenous nucleic acid sequence, which is capable of being amplified in the presence of a compatible RdRP. In some embodiments, the amplification is self-limiting. In certain embodiments, the amount of polymerase is diluted over time (e.g., due to degradation of the RdRP and/or mRNA encoding the RdRP, or by replication of the cell without replenishment of the RdRP or mRNA encoding the RdRP), thereby reducing amplification over time. In some embodiments, the nucleic acid construct is capable of self-replicating. In some embodiments, the nucleic acid construct does not undergo self-replication.

In some embodiments, the nucleic acid construct comprises a gene of interest (e.g., encoding an exogenous protein or a functional RNA). In some embodiments, the gene of interest is in the sense orientation relative to the 5′ UTR. In some embodiments, the gene of interest is in the antisense orientation relative to the 5′ UTR. In some embodiments, the nucleic acid construct comprises a plurality of genes of interest (e.g., 2, 3, 4, 5, 6, 7, 8, 9, or 10 genes of interest). In some embodiments, one or more of (e.g., each of) the plurality of genes of interest is a different gene (e.g., encoding a different protein or functional RNA molecule). In some embodiments, two genes of interest are separated by a nucleic acid sequence comprising an IRES sequence (e.g., as described herein) and/or a 2A sequence (e.g., as described herein), e.g., a T2A sequence. In some embodiments, there is no IRES sequence (e.g., as described herein) and/or there is no 2A sequence (e.g., as described herein), e.g., a T2A sequence between the two genes of interest.

In some embodiments, a first gene of interest is oriented as a sense strand relative to the 5′ UTR and a second gene of interest is in the sense orientation relative to the 5′ UTR. In some embodiments, a first gene of interest is in the sense orientation relative to the 5′ UTR and a second gene of interest is in the antisense orientation relative to the 5′ UTR. In some embodiments, a first gene of interest is oriented in the antisense orientation relative to the 5′ UTR and a second gene of interest is in the sense orientation strand relative to the 5′ UTR. In some embodiments, a first gene of interest is in the antisense orientation relative to the 5′ UTR and a second gene of interest is in the antisense orientation relative to the 5′ UTR. In certain embodiments, the first gene of interest and the second gene of interest are comprised in a positive strand RNA construct, e.g., as described herein. In other embodiments, the first gene of interest and the second gene of interest are comprised in a negative strand RNA construct. e.g., as described herein.

In certain embodiments, the nucleic acid construct is a positive strand RNA construct, the first gene of interest is in the sense orientation relative to the 5′ UTR, and the second gene of interest is in the antisense orientation relative to the 5′ UTR. e.g., such that the first gene of interest can be expressed from the positive strand RNA construct, and the second gene of interest is only expressed from a corresponding negative strand RNA produced from the positive strand RNA construct, e.g., by an RdRP. In other embodiments, the nucleic acid construct is a negative strand RNA construct, the first gene of interest is in the sense orientation, and the second gene of interest is in the antisense orientation, e.g., such that the second gene of interest can be expressed from the negative strand RNA construct, and the second gene of interest is only expressed from a corresponding positive strand RNA produced from the negative strand RNA construct, e.g., by an RdRP.

In some embodiments, the nucleic acid construct further encodes a compatible RdRP. For example, the nucleic acid construct may comprise an open reading frame encoding a gene of interest (e.g., an exogenous protein, e.g., comprising a therapeutic agent) and an open reading frame encoding the compatible RdRP. In some embodiments, the sequences encoding the gene of interest and the compatible RdRP comprise an IRES, e.g., disposed between the gene of interest and the compatible RdRP. In some embodiments, the compatible RdRP is supplied to a cell in trans. e.g., by delivering a second nucleic acid construct encoding the RdRP to the cell, by integrating an RdRP-encoding gene into the genome of the cell, or by providing the RdRP to the cell as a purified protein (e.g., as described below).

In certain embodiments, the nucleic acid construct is an RNA construct as described herein, which can be introduced into the cell, e.g., by electroporation or in a lipid nanoparticle. In certain embodiments, the nucleic acid construct is a DNA (e.g., a vector, e.g., a viral vector, e.g., a lentiviral vector) comprising a nucleic acid sequence encoding an RNA construct as described herein, which DNA can be introduced into the cell, e.g., by electroporation, transfection, or in a lipid nanoparticle. A DNA vector can also be used as a template for an in vitro transcription reaction, to create RNA suitable to be introduced into a cell.

In some embodiments, a nucleic acid construct described herein lacks at least one functional flavivirus gene or a fragment of the gene. In some embodiments, a nucleic acid construct that lacks at least one functional flavivirus gene comprises a nonfunctional mutant or variant of the gene, e.g., an enzymatically inactive mutant or variant, or a mutant or variant that is ineffective for viral assembly. In some embodiments, the nucleic acid construct does not comprise any sequences encoding a functional flaviviral structural protein. In some embodiments, the nucleic acid construct does not comprise any sequences encoding a functional flaviviral nonstructural protein. In embodiments, the nucleic acid construct does not comprise any sequences encoding a functional flaviviral structural protein or any sequences encoding a functional flaviviral nonstructural protein. In some embodiments, the only functional flaviviral nonstructural protein encoded by the nucleic acid construct is a gene encoding an RdRP compatible with the 5′ UTR of the nucleic acid construct. In some embodiments, the nucleic acid construct encodes a helicase protein (e.g., a viral helicase, e.g., a flavivirus NS3 protein, e.g., as described herein, or a protein having at least 75%, 80%, 85%, 90%, 95%, 96%, 97%, 98%, 99%, or 100% sequence identity thereto). In certain embodiments, the helicase protein comprises a Dengue virus NS3 protein, e.g., as described herein, or a protein having at least 75%, 80%, 85%, 90%, 95%, 96%, 97%, 98%, 99%, or 100% sequence identity thereto. In certain embodiments, the helicase protein comprises a WNV NS3 protein, e.g., as described herein, or a protein having at least 75%, 80%, 85%, 90%, 95%, 96%, 97%, 98%, 99%, or 100% sequence identity thereto. In certain embodiments, the helicase protein comprises a SLEV NS3 protein, e.g., as described herein, or a protein having at least 75%, 80%, 85%, 90%, 95%, 96%, 97%, 98%, 99%, or 100% sequence identity thereto. In some embodiments, the helicase protein is not a flavivirus helicase protein. In certain embodiments, the nucleic acid construct encodes both the helicase gene and the compatible RdRP. In certain embodiments, the helicase is comprised in another protein encoded by the nucleic acid construct, e.g., the exogenous protein (e.g., the therapeutic agent) and/or the RdRP. In certain embodiments, the helicase is a domain of the other protein encoded by the nucleic acid construct.

The nucleic acid construct (e.g., RNA construct) may comprise a plurality of, e.g., two, CS regions (e.g., as described in Gebhard et al. (2011) Viruses 3: 1739-1756 and/or as described herein), which are involved in circularization of the nucleic acid construct and which allow for the cyclization RdRP amplification of the construct. Generally, such a nucleic acid will comprise a 5′ CS region and a 3′ CS region capable of hybridizing to (e.g., complementary to) the 5′ CS region. In some embodiments the 5′ CS region and the 3′ CS region are perfectly complementary. In some embodiments the 5′ CS region and the 3′ CS region have one or more mismatch (e.g., bulges or gaps) but are still capable of hybridizing to each other. In some embodiments, the RdRP-responsive 5′ UTR comprises the 5′ CS region. In some embodiments, the RdRP-responsive 5′ UTR does not comprise the 5′ CS region. In certain embodiments, the RdRP-responsive 5′ UTR is located 5′ to an open reading frame encoding an exogenous protein (e.g., comprising a therapeutic agent), wherein the open reading frame comprises the 5′ CS region. In some embodiments, the 5′ CS region is native to the RdRP-responsive 5′ UTR. In some embodiments, the 5′ CS region is synthetic (e.g., not naturally-occurring). For instance, a pair of CS regions (e.g., a 5′ CS region and a 3′ CS region) can be designed such that both regions have sufficient complementarity to each other and are capable of hybridizing with each other to facilitate the circularization of an RNA construct. In some embodiments, each CS region is at least about 7 nucleotide in length (e.g., 7, 8, 9, 10, 11, 12, 13, 14, 15, 16, 17, 18, 19, 20, or more nucleotides in length). In certain embodiments, the RdRP-responsive 5′ UTR comprises a 5′ CS region. In some embodiments, the 5′ CS region is native to the RdRP-responsive 5′ UTR. In some embodiments, the 5′CS region is synthetic. In certain embodiments, the RdRP-responsive 3′ UTR comprises a 3′ CS region. In certain embodiments, the 3′ CS region is located in Domain III of the RdRP-responsive 3′ UTR (e.g., in CS1 of the RdRP-responsive 3′ UTR), e.g., as described in Gebhard et al. 2011, supra. In some embodiments, the 3′ CS region is native to the RdRP-responsive 3′ UTR. In some embodiments, the 3′ CS region is synthetic.

### Flavivirus-Derived Constructs

In some embodiments, the nucleic acid construct (e.g., RNA construct) comprises one or more nucleic acid sequences derived from a flavivirus sequence. In some embodiments, the flavivirus is selected from Dengue virus, West Nile virus (WNV), Saint Louis encephalitis virus (SLEV), yellow fever virus (YFV), and Murray Valley encephalitis virus (MVEV), e.g., comprising a sequence (e.g., an RdRP, helicase, 5′ UTR, or 3′ UTR sequence, or a functional portion thereof) as described herein, or a sequence having at least 75% (e.g., at least 75%, 80%, 85%, 90%, 95%, 96%, 97%, 98%, 99%, or 100%) sequence identity thereto. In some embodiments, the nucleic acid construct comprises a plurality of nucleic acid sequences derived from a single flavivirus (e.g., Dengue virus, West Nile virus (WNV), Saint Louis encephalitis virus (SLEV), yellow fever virus (YFV), and Murray Valley encephalitis virus (MVEV)). In some embodiments, the nucleic acid construct comprises a plurality of nucleic acid sequences derived from more than one flavivirus.

In some embodiments, the nucleic acid construct comprises a flavivirus 5′ UTR (e.g., as described herein). In some embodiments, the nucleic acid construct comprises a 5′ CS region. (e.g., as described in Gebhard et al. (2011) Viruses 3: 1739-1756), or a sequence derived therefrom (e.g., a sequence having at least 80%, 85%, 90%, 95%, 96%, 97%, 98%, 99%, or 100% sequence identity thereto). In some embodiments, the flavivirus 5′ UTR comprises the 5′ CS region. In some embodiments, the flavivirus 5′ UTR does not comprise the 5′ CS region. In certain embodiments, the flavivirus 5′ UTR is located 5′ to an open reading frame encoding an exogenous protein (e.g., comprising a therapeutic agent), wherein the open reading frame comprises a flavivirus 5′ CS region. In certain embodiments, the flavivirus 5′ CS region is located within 30 nucleotides (e.g., within 5, 10, 11, 12, 13, 14, 15, 16, 17, 18, 19, 20, 21, 22, 23, 24, 25, or 30 nucleotides) of the 5′ end of the open reading frame. In certain embodiments, the flavivirus 5′ CS region is located within nucleotides 13-16 of the open reading frame. In some embodiments, the flavivirus 5′ CS region is capable of hybridizing to (e.g., complementary to) a flavivirus 3′ CS region comprised in the nucleic acid construct (e.g., in a 3′ UTR of the nucleic acid construct). In certain embodiments, the flavivirus 5′ CS region is derived from a Dengue virus, West Nile virus (WNV), Saint Louis encephalitis virus (SLEV), yellow fever virus (YFV), or Murray Valley encephalitis virus (MVEV) 5′ CS region. In certain embodiments, the flavivirus 5′ CS region is derived from a Dengue virus 5′ CS region.

In some embodiments, the nucleic acid construct comprises a flavivirus 3′ UTR (e.g., as described herein). In some embodiments, the nucleic acid construct comprises a 3′ CS region (e.g., as described in Gebhard et al. (2011) Viruses 3: 1739-1756), or a sequence derived therefrom (e.g., a sequence having at least 80%, 85%, 90%, 95%, 96%, 97%, 98%, 99%, or 100% sequence identity thereto). In certain embodiments, the flavivirus 3′ UTR comprises the flavivirus 3′ CS region. In certain embodiments, the flavivirus 3′ CS is located in Domain III of the flavivirus 3′ UTR (e.g., in CS1 of the flavivirus 3′ UTR), e.g., as described in Gebhard et al. 2011, supra. In some embodiments, the flavivirus 3′ CS region is capable of hybridizing to (e.g., complementary to) a flavivirus 5′ CS region comprised in the nucleic acid construct (e.g., in an open reading frame of the nucleic acid construct). In certain embodiments, the flavivirus 3′ CS region is derived from a Dengue virus, West Nile virus (WNV), Saint Louis encephalitis virus (SLEV), yellow fever virus (YFV), or Murray Valley encephalitis virus (MVEV) 3′ CS region. In certain embodiments, the flavivirus 5′ CS region is derived from a Dengue virus 3′ CS region.

### Dengue Virus-Derived Constructs

In some embodiments, the flavivirus comprises Dengue virus. Dengue virus is a flavivirus whose genome encodes three structural proteins (C, prM, and E) and five nonstructural proteins (NS1, NS2, NS3, NS4, and NS5), NS5 is the Dengue virus RdRP. Four Dengue virus serotypes are commonly known, referred to as DEN-1. DEN-2. DEN-3, and DEN-4. Any of the Dengue virus-derived constructs described herein may include Dengue virus sequences derived from one or more of serotypes DEN-1, DEN-2, DEN-3, and DEN-4. Any of the Dengue virus-derived constructs described herein may lack genes encoding functional structural proteins of the Dengue virus genome (e.g., C, prM, and E). Any of the Dengue virus-derived constructs described herein may lack genes encoding functional nonstructural proteins of the Dengue virus genome (e.g., NS1, NS2, NS3, NS4, and NS5).

Exemplary Dengue virus-derived RdRP amino acid sequences, RdRP-responsive 5′UTR nucleic acid sequences, and RdRP-responsive 3′UTR nucleic acid sequences are provided below in Table 1. In some embodiments, the nucleic acid construct comprises a nucleic acid sequence having at least 75% (e.g., at least 75%, 80%, 85%, 90%, 95%, 96%, 97%, 98%, 99%, or 100%) sequence identity to a Dengue virus RdRP-responsive 5′UTR sequence (e.g., as listed in Table 1). In some embodiments, the nucleic acid construct comprises a nucleic acid sequence having at least 75% (e.g., at least 75%, 80%, 85%, 90%, 95%, 96%, 97%, 98%, 99%, or 100%) sequence identity to a truncated Dengue virus RdRP-responsive 5′UTR sequence (e.g., as listed in Table 1). In some embodiments, the nucleic acid construct comprises a nucleic acid sequence having at least 75% (e.g., at least 75%, 80%, 85%, 90%, 95%, 96%, 97%, 98%, 99%, or 100%) sequence identity to a Dengue virus RdRP-responsive 3′UTR sequence (e.g., as listed in Table 1). In some embodiments, the nucleic acid construct comprises a nucleic acid sequence having at least 75% (e.g., at least 75%, 80%, 85%, 90%, 95%, 96%, 97%, 98%, 99%, or 100%) sequence identity to a truncated Dengue virus RdRP-responsive 3′UTR sequence (e.g., as listed in Table 1). In some embodiments, the nucleic acid construct comprises a nucleic acid sequence having at least 75% (e.g., at least 75%, 80%, 85%, 90%, 95%, 96%, 97%, 98%, 99%, or 100%) sequence identity to a Dengue virus 5′ CS region (e.g., as listed in Table 1). In some embodiments, the nucleic acid construct comprises a nucleic acid sequence having at least 75% (e.g., at least 75%, 80%, 85%, 90%, 95%, 96%, 97%, 98%, 99%, or 100%) sequence identity to a Dengue virus 3′ CS region (e.g., as listed in Table 1). In some embodiments, the nucleic acid construct comprises a nucleic acid sequence having at least 75% (e.g., at least 75%, 80%, 85%, 90%, 95%, 96%, 97%, 98%, 99%, or 100%) sequence identity to a positive strand Dengue virus RdRP-responsive 5′UTR sequence (e.g., as listed in Table 1). In some embodiments, the nucleic acid construct comprises a nucleic acid sequence having at least 75% (e.g., at least 75%, 80%, 85%, 90%, 95%, 96%, 97%, 98%, 99%, or 100%) sequence identity to a positive strand Dengue virus RdRP-responsive 3′UTR sequence (e.g., as listed in Table 1). In some embodiments, the nucleic acid construct comprises a nucleic acid sequence having at least 75% (e.g., at least 75%, 80%, 85%, 90%, 95%, 96%, 97%, 98%, 99%, or 100%) sequence identity to a negative strand Dengue virus RdRP-responsive 5′UTR sequence (e.g., as listed in Table 1). In some embodiments, the nucleic acid construct comprises a nucleic acid sequence having at least 75% (e.g., at least 75%, 80%, 85%, 90%, 95%, 96%, 97%, 98%, 99%, or 100%) sequence identity to a negative strand Dengue virus RdRP-responsive 3′UTR sequence (e.g., as listed in Table 1). In some embodiments, the nucleic acid construct comprises a nucleic acid sequence having at least 75% (e.g., at least 75%, 80%, 85%, 90%, 95%, 96%, 97%, 98%, 99%, or 100%) sequence identity to a Dengue virus NS5 sequence (e.g., as listed in Table 1), or the reverse complement thereof. In some embodiments, the nucleic acid construct comprises a nucleic acid sequence having at least 75% (e.g., at least 75%, 80%, 85%, 90%, 95%, 96%, 97%, 98%, 99%, or 100%) sequence identity to a Dengue virus NS3 sequence (e.g., as listed in Table 1), or the reverse complement thereof.

TABLE 1
Exemplary Dengue virus sequences

| Description | Sequence | SEQ ID NO: |
|---|---|---|
| Dengue virus | AGTTGTTAGTCTACGTGGACCGACAAGAACAGTTT | 5 |
| RdRP-responsive | CGAATCGGAAGCTTGCTTAACGTAGTTCTGACAGT | |
| 5′uTR (positive | TTTTTATTTAGAGAGCAGATCTCTG | |
| strand) | | |
| Dengue virus | TCACAAAACAAAGGAAAATAAGAAATCAAACAAG | 6 |
| RdRP-responsive | GCAAGAAGTCAGGCCGGATTAAGCCATAGTACGGT | |
| 3′ UTR (positive | AAGAGCTATGCTGCCTGTGAGCCCCGTCTAAGGAC | |
| strand) | GTAAAATGAAGTCAGGCCGGAAGCCACGGTTTGA | |
| | GCAAACCGTGCTGCCTGTAGCTCCATCGTGGGGAT | |
| | GTAAAAACCCGGGAGGCTGCAACCCATGGAAGCT | |
| | GTACGCATGGGGTAGCAGACTAGTGGTTAGAGGA | |
| | GACCCCTCCCAAAACACAACGCAGCAGCGGGGCC | |
| | CAACACCAGGGGAAGCTGTACCCTGGTGGTAAGG | |
| | ACTAGAGGTTAGAGGAGACCCCCCGCACAACAAT | |
| | AAACAGCATATTGACGATGGGAGAGACCAGAGAT | |
| | CCTGCTGTCTCTACAGCATCATTCCAGGCACAGAA | |
| | CGCCAGAAAATGGAATGGTGCTGTTGAATCAACAG | |
| | GTTCT | |
| Dengue virus RdRP | TCAACAATCAGATGCACCTGGCTGTTCTTGTCAAA | 7 |
| responsive 5′ UTR | GCTTAGCCTTCGAACGAATTGCATCAAGACTGTCA | |
| (negative strand) | AAAAATAAATCTCTCGTCTAGAGAC | |
| Dengue virus RdRP | AGTGTTTTGTTTCCTTTTATTCTTTAGTTTGTTCCGT | 8 |
| responsive 3′ UTR | TCTTCAGTCCGGCCTAATTCGGTATCATGCCATTCT | |
| (negative strand) | CGATACGACGGACACTCGGGGCAGATTCCTGCATT | |
| | TTACTTCAGTCCGGCCTTCGGTGCCAAACTCGTTTG | |
| | GCACGACGGACATCGAGGTAGCACCCCTACATTTT | |
| | TGGGCCCTCCGACGTTGGGTACCTTCGACATGCGT | |
| | ACCCCATCGTCTGATCACCAATCTCCTCTGGGGAG | |
| | GGTTTTGTGTTGCGTCGTCGCCCCGGGTTGTGGTCC | |
| | CCTTCGACATGGGACCACCATTCCTGATCTCCAATC | |
| | TCCTCTGGGGGGCGTGTTGTTATTTGTCGTATAACT | |
| | GCTACCCTCTCTGGTCTCTAGGACGACAGAGATGT | |
| | CGTAGTAAGGTCCGTGTCTTGCGGTCTTTTACCTTA | |
| | CCACGACAACTTAGTTGTCCAAGA | |
| Dengue virus NS5 | GTGNIGETLGEKWKSRLNALGKSEFQIYKKSGIQEVD | 9 |
| RdRP, (GenBank | RTLAKEGIKRGETDHHAVSRGSAKLRWFVERNMVTP | |
| Accession No. | EGKVVDLGCGRGGWSYYCGGLKNVREVKGLTKGG | |
| NP_739590) | PGHEEPIPMSTYGWNLVRLQSGVDVFFIPPEKCDTLL | |
| | CDIGESSPNPTVEAGRILRVLNLVENWLNNMFQFCIK | |
| | VLNPYMPSVIEKMEALQRKYGGALVRNPLSRNSTHE | |
| | MYWVSNASGNIVSSVNMISRMLINRFTMRYKKATYE | |
| | PDVDLGSGTRNIGIESEIPNLDIIGKRIEKIKQEHETSW | |
| | HYDQDHPYKTWAYHGSYETKQTGSASSMVNGVVRL | |
| | LTKPWDVVPMVTQMAMTDTTPFGQQRVFKEKVDTR | |
| | TQEPKEGTKKLMKITAEWLWKELGKKKTPRMCTRE | |
| | EFTRKVRSNAALGAIFTDENKKSAREAVEDSRFWE | |
| | LVDKERNLHLEGKCETCVYNMMGKREKKLGEFGKA | |
| | KGSRAIWYMWLGARFLEFEALGFLNEDHWFSRENSL | |
| | SGVEGEGLHKLGYILRDVSKKEGGAMYADDTAGWD | |
| | TRITLEDLKNEEMVTNHMEGEHKKLAEAIFKLTYQN | |
| | KVVRVQRPTPRGTVMDIISRRDQRGSGQVGTYGLNT | |
| | FTNMEAQLIRQMEGEGVFKSIQHLTITEEIAVQNWLA | |
| | RVGRERLSRMAISGDDCVVKPLDDRFASALTALNDM | |
| | GKIRKDIQQWEPSRGWNDWTQVPFCSHHEHELIMKD | |
| | GRVLVVPCRNQDELIGRARISQGAGWSLRETACLGK | |
| | SYAQMWSLMYFHRRDLRLAANAICSAVPSHWVPTS | |
| | RTTWSIHAKHEWMTTEDMLTVWNRVWIQENPWME | |
| | DKTPVESWEEIPYLGKREDQWCGSLIGLTSRATWAK | |
| | NIQAAINQVRSLIGNEEYTDYMPSMKRFRREEEEAGV | |
| | LW | |
| Truncated Dengue | ACAAAGACAGATTCTTTGAGGGAGCTAAGCTTAAC | 10 |
| Virus Type II | GTAGTTCTAACAGTTTTTTAATTAGAGAGCAGATCT | |
| RdRP-responsive | CTGATGAACAACCAACGGAAAAAGGCGAGAAATA | |
| 5′ UTR with CS | CGCCTTTCAATATGCTG | |
| (truncated to | | |
| exclude nucleotides | | |
| downstream from | | |
| the cyclization | | |
| sequence (CS); | | |
| * CS underlined) | | |
| Truncated Dengue | ACAAAGACAGATTCTTTGAGGGAGCTAAGCTTAAC | 11 |
| Virus Type II | GTAGTTCTAACAGTTTTTTAATTAGAGAGCAGATCT | |
| RdRP-responsive | CTGATGAACAACCAACGGAAAAAGGCGAGAAATA | |
| 5′ UTR (truncated | CGCCTT | |
| to exclude | | |
| nucleotides | | |
| beginning at the | | |
| CS) | | |
| Truncated Dengue | CAGCATATTGACGCTGGGAAAGACCAGAGATCCTG | 12 |
| Virus Type II | CTGTCTCCTCAGCATCATTCCAGGCACAGAACGCC | |
| RdRP-responsive | AGAAAATGGAATGGTGTTGT | |
| 3′ UTR with CS | | |
| (truncated to | | |
| exclude nucleotides | | |
| upstream from the | | |
| CS; * CS | | |
| underlined) | | |
| Truncated Dengue | CGCTGGGAAAGACCAGAGATCCTGCTGTCTCCTCA | 13 |
| Virus Type II | GCATCATTCCAGGCACAGAACGCCAGAAAATGGA | |
| RdRP-responsive | ATGGTGTTGT | |
| 3′ UTR (truncated | | |
| to exclude | | |
| nucleotides | | |
| beginning at the | | |
| CS) | | |
| Full length Dengue | ACAAAGACAGATTCTTTGAGGGAGCTAAGCTTAAC | 14 |
| Virus Type II | GTAGTTCTAACAGTTTTTTAATTAGAGAGCAGATCT | |
| RdRP-responsive | CTGATGAACAACCAACGGAAAAAGGCGAGAAATA | |
| 5′ UTR | CGCCTTTCAATATGCTGAAACGCGAGAGAAACCGC | |
| | GTGTCGACTGTGCAACAGTT | |
| Full Length Dengue | GAAGGCAGAATTAACATGAGATAAGATTAGAAGT | 15 |
| Virus Type II | CAGGTCGCATTAAGCCATAGTACGGGAAAAACTAT | |
| RdRP-responsive | GCTACCTGTGAGCCCCGTCCAAGGACGTTAAAAGA | |
| 3′ UTR | AGTCAGGCCACTACAAGTGCCATAACTTGAGCAAA | |
| | CTATGCAGCCTGTAGCTCCACCTGGGAAGGTGTAA | |
| | AAAATCTGGGAGGCCACAAACCATGGAAGCTGTA | |
| | CGCATGGCGTAGTGGACTAGCGGTTAGAGGAGACC | |
| | CCTCCCCTGCAAATCGCAGCAACAATGGGGGCCCA | |
| | AGGTGAGATGAAGCTGTAGTCTCACTGGAAGGACT | |
| | AGAGGTAGAGGAGACCCCCCCGAAACAAAAAAC | |
| | AGCATATTGACGCTGGGAAAGACCAGAGATCCTGC | |
| | TGTCTCCTCAGCATCATTCCAGGCACAGAACGCCA | |
| | GAAAATGGAATGGTGTTGT | |
| Full Length Dengue | AGVLWDVPSPPPVGKAELEDGAYRIKQKGILGYSQIG | 16 |
| Virus Type II NS3 | AGVYKEGTFHTMWHVTRGAVLMTIKGKRIEPSWAD | |
| (helicase) amino | VRKDLISYGGGWKLEGEWKEGEEVQVLALEPGKNP | |
| acid sequence | RAVQTKPGLFKTDTGTIGAVSLDFSPGTSGSPIVDKK | |
| | GKVVGLYGNGVVTRSGTYVSAIAQTEKSIEDNPEIED | |
| | DIFRKRRLTIMDLHPGAGKTKRYLPAIVREAIKRGLR | |
| | TLILAPTRVVAAEMEEALRGLPIRYQTPAIRAEHTGRE | |
| | IVDLMCHATFTMRLLSPVRVPNYNLIIMDEAHFTDPA | |
| | SIAARGYISTRVEMGEAAGIFMTATPPGSRDPFPQSN | |
| | APIMDEEREIPERSWNSGHEWVTDFKGKTVWFVPSIK | |
| | AGNDIAACLRKNGKKVIQLSRKTFDSEYIKTRTNDW | |
| | DFVVTTDISEMGANFKAERVIDPRRCMKPVILTDGEE | |
| | RVILAGPMPVTHSSAAQRRGRIGRNPKNENDQYIYM | |
| | GEPLENDEDCAHWKEAKMLLDNINTPEGIIPSMFEPE | |
| | REHKVDAIDGEYRLRGEARKTFVDLMRRGDLPVWLA | |
| | YRVAAEGINYADRRWCFDGVKNNQILEENNEVEIWT | |
| | KEGERKKLKPRWLDARIYSDPLALKEFKEFAAGRK | |

In some aspects, the present disclosure provides a composition comprising:

(i) a first nucleic acid molecule comprising a Dengue Virus 5′ UTR (e.g., having at least 75%, 80%, 85%, 90%, 95%, 96%, 97%, 98%, 99%, or 100% sequence identity to SEQ ID NO: 5, 7, 10, 11, or 14) and an exogenous nucleic acid sequence; wherein the first nucleic acid molecule does not comprise a sequence encoding a functional RNA-dependent RNA polymerase (RdRP), and

(ii) a second nucleic acid molecule comprising a sequence encoding a compatible RdRP;

wherein the first nucleic acid molecule and the second nucleic acid molecule are each non-naturally occurring nucleic acid molecules. In some embodiments, the first nucleic acid comprises a Dengue Virus 3′ UTR (e.g., having at least 75%, 80%, 85%, 90%, 95%, 96%, 97%, 98%, 99%, or 100% sequence identity to SEQ ID NO: 6, 8, 12, 13, or 15).

In some embodiments, the nucleic acid construct is an RNA construct comprising a Dengue virus RdRP-responsive 5′ UTR and an exogenous nucleic acid sequence (e.g., a sequence encoding at least one exogenous gene). In some embodiments, the Dengue virus RdRP-responsive 5′UTR comprises or consists of the nucleic acid sequence of SEQ ID NO: 5 or 7. Such an RNA construct can undergo amplification in the presence of a compatible RdRP. e.g., in an erythroid cell expressing the compatible RdRP. In some embodiments, the amplification is self-limiting. In certain embodiments, the amount of polymerase is diluted over time (e.g., due to degradation of the RdRPs or mRNAs), thereby reducing amplification over time. In some embodiments, the RNA construct comprises a Dengue virus RdRP-responsive 5′ UTR and the cell comprises a compatible Dengue virus RdRP (e.g., encoded by a Dengue virus NS5 gene in the RNA construct or in a second RNA construct present in the cell). In some embodiments, the RNA construct comprises a Dengue virus RdRP-responsive 3′ UTR. In some embodiments, the Dengue virus RdRP responsive 3′UTR comprises or consists of the nucleic acid sequence of SEQ ID NO: 6 or 8. In some embodiments, the RNA construct comprises a Dengue virus 5′ CS region and a Dengue virus 3′ CS region capable of hybridizing to (e.g., complementary to) the 5′ CS region. Embodiments in which the RNA construct comprises both a Dengue virus 5′ UTR and a Dengue virus 3′ UTR can undergo RdRP-mediated RNA synthesis in both the positive strand and negative strand forms. e.g., thereby resulting in exponential amplification of the construct. Embodiments in which the RNA construct comprises only a Dengue virus 5′ UTR can only undergo RdRP-mediated RNA synthesis in the negative strand form, e.g., thereby resulting in linear amplification of the construct.

A Dengue virus-based RNA construct generally comprises one or more of the following elements, arranged in a variety of orientations and orders: Dengue virus 5′ UTR, exogenous nucleic acid sequence. Dengue virus 3′ UTR, and/or gene encoding Dengue virus RdRP. In some embodiments, the Dengue virus-based RNA construct does not encode a functional Dengue virus structural protein. In some embodiments, the Dengue virus-based RNA construct does not encode a functional Dengue virus nonstructural protein. In some embodiments, the Dengue virus-based RNA construct does not encode a functional Dengue virus structural protein or a functional Dengue virus nonstructural protein.

### Other Flavivirus-Derived Constructs

In some embodiments, a nucleic acid construct provided herein comprises sequences derived from a flavivirus selected from West Nile virus (WNV), Saint Louis encephalitis virus (SLEV), yellow fever virus (YFV), and Murray Valley encephalitis virus (MVEV). Exemplary nucleotide and amino acid sequences that can be used to generate the systems described herein (e.g., RdRP amino acid sequences, helicase amino acid sequences, RdRP-responsive 5′ UTRs, and/or RdRP-responsive 3′ UTR sequence) are provided at Tables 2-5 below. The sequences in Tables 2-5, sequences having at least 75% (e.g., at least 75%, 80%, 85%, 90%, 95%, 96%, 97%, 98%, 99%, or 100%) sequence identity thereto may be used as described herein.

In some aspects, the present disclosure provides a composition comprising:

(i) a first nucleic acid molecule comprising a West Nile Virus 5′ UTR (e.g., having at least 75%, 80%, 85%, 90%, 95%, 96%, 97%, 98%, 99%, or 100% sequence identity to SEQ ID NO: 19 or 20) and an exogenous nucleic acid sequence; wherein the first nucleic acid molecule does not comprise a sequence encoding a functional RNA-dependent RNA polymerase (RdRP), and

(ii) a second nucleic acid molecule comprising a sequence encoding a compatible RdRP;

wherein the first nucleic acid molecule and the second nucleic acid molecule are each non-naturally occurring nucleic acid molecules. In some embodiments, the first nucleic acid comprises a West Nile Virus 3′ UTR (e.g., having at least 75%, 80%, 85%, 90%, 95%, 96%, 97%, 98%, 99%, or 100% sequence identity to SEQ ID NO: 21 or 22).

In some aspects, the present disclosure provides a composition comprising:

(i) a first nucleic acid molecule comprising a Saint Louis Encephalitis Virus 5′ UTR (e.g., having at least 75%, 80%, 85%, 90%, 95%, 96%, 97%, 98%, 99%, or 100% sequence identity to SEQ ID NO: 25 or 26) and an exogenous nucleic acid sequence; wherein the first nucleic acid molecule does not comprise a sequence encoding a functional RNA-dependent RNA polymerase (RdRP), and

(ii) a second nucleic acid molecule comprising a sequence encoding a compatible RdRP;

wherein the first nucleic acid molecule and the second nucleic acid molecule are each non-naturally occurring nucleic acid molecules. In some embodiments, the first nucleic acid comprises an SLEV 3′ UTR (e.g., having at least 75%, 80%, 85%, 90%, 95%, 96%, 97%, 98%, 99%, or 100% sequence identity to SEQ ID NO: 27 or 28).

In some aspects, the present disclosure provides a composition comprising:

(i) a first nucleic acid molecule comprising a Yellow Fever Virus 5′ UTR (e.g., having at least 75%, 80%, 85%, 90%, 95%, 96%, 97%, 98%, 99%, or 100% sequence identity to SEQ ID NO: 30 or 31) and an exogenous nucleic acid sequence; wherein the first nucleic acid molecule does not comprise a sequence encoding a functional RNA-dependent RNA polymerase (RdRP), and

(ii) a second nucleic acid molecule comprising a sequence encoding a compatible RdRP;

wherein the first nucleic acid molecule and the second nucleic acid molecule are each non-naturally occurring nucleic acid molecules. In some embodiments, the first nucleic acid comprises a Yellow Fever Virus 3′ UTR (e.g., having at least 75%, 80%, 85%, 90%, 95%, 96%, 97%, 98%, 99%, or 100% sequence identity to SEQ ID NO: 32 or 33).

In some aspects, the present disclosure provides a composition comprising:

(i) a first nucleic acid molecule comprising a Murray Valley Encephalitis Virus 5′ UTR (e.g., having at least 75%, 80%, 85%, 90%, 95%, 96%, 97%, 98%, 99%, or 100% sequence identity to SEQ ID NO: 35 or 36) and an exogenous nucleic acid sequence; wherein the first nucleic acid molecule does not comprise a sequence encoding a functional RNA-dependent RNA polymerase (RdRP), and

(ii) a second nucleic acid molecule comprising a sequence encoding a compatible RdRP;

wherein the first nucleic acid molecule and the second nucleic acid molecule are each non-naturally occurring nucleic acid molecules. In some embodiments, the first nucleic acid comprises a Murray Valley Encephalitis Virus 3′ UTR (e.g., having at least 75%, 80%, 85%, 90%, 95%, 96%, 97%, 98%, 99%, or 100% sequence identity to SEQ ID NO: 37 or 38).

In some embodiments, the nucleic acid construct comprises a nucleic acid sequence having at least 75% (e.g., at least 75%, 80%, 85%, 90%, 95%, 96%, 97%, 98%, 99%, or 100%) sequence identity to a WNV 5′ UTR sequence (e.g., as listed in Table 2), or the reverse complement thereof. In some embodiments, the nucleic acid construct comprises a nucleic acid sequence having at least 75% (e.g., at least 75%, 80%, 85%, 90%, 95%, 96%, 97%, 98%, 99%, or 100%) sequence identity to a truncated WNV 5′ UTR sequence (e.g., as listed in Table 2), or the reverse complement thereof. In some embodiments, the nucleic acid construct comprises a nucleic acid sequence having at least 75% (e.g., at least 75%, 80%, 85%, 90%, 95%, 96%, 97%, 98%, 99%, or 100%) sequence identity to a WNV 3′ UTR sequence (e.g., as listed in Table 2), or the reverse complement thereof. In some embodiments, the nucleic acid construct comprises a nucleic acid sequence having at least 75% (e.g., at least 75%, 80%, 85%, 90%, 95%, 96%, 97%, 98%, 99%, or 100%) sequence identity to a truncated WNV 3′ UTR sequence (e.g., as listed in Table 2), or the reverse complement thereof. In some embodiments, the nucleic acid construct comprises a nucleic acid sequence having at least 75% (e.g., at least 75%, 80%, 85%, 90%, 95%, 96%, 97%, 98%, 99%, or 100%) sequence identity to a WNV 5′ CS region (e.g., as listed in Table 2). In some embodiments, the nucleic acid construct comprises a nucleic acid sequence having at least 75% (e.g., at least 75%, 80%, 85%, 90%, 95%, 96%, 97%, 98%, 99%, or 100%) sequence identity to a WNV 3′ CS region (e.g., as listed in Table 2). In some embodiments, the nucleic acid construct comprises a nucleic acid sequence having at least 75% (e.g., at least 75%, 80%, 85%, 90%, 95%, 96%, 97%, 98%, 99%, or 100%) sequence identity to a WNV NS3 sequence (e.g., as listed in Table 2), or the reverse complement thereof. In some embodiments, the nucleic acid construct comprises a nucleic acid sequence having at least 75% (e.g., at least 75%, 80%, 85%, 90%, 95%, 96%, 97%, 98%, 99%, or 100%) sequence identity to a WNV NS5 sequence (e.g., as listed in Table 2), or the reverse complement thereof.

In some embodiments, the nucleic acid construct comprises a nucleic acid sequence having at least 75% (e.g., at least 75%, 80%, 85%, 90%, 95%, 96%, 97%, 98%, 99%, or 100%) sequence identity to a truncated SLEV 5′ UTR sequence (e.g., as listed in Table 3), or the reverse complement thereof. In some embodiments, the nucleic acid construct comprises a nucleic acid sequence having at least 75% (e.g., at least 75%, 80%, 85%, 90%, 95%, 96%, 97%, 98%, 99%, or 100%) sequence identity to a truncated SLEV 3′ UTR sequence (e.g., as listed in Table 3), or the reverse complement thereof. In some embodiments, the nucleic acid construct comprises a nucleic acid sequence having at least 75% (e.g., at least 75%, 80%, 85%, 90%, 95%, 96%, 97%, 98%, 99%, or 100%) sequence identity to a SLEV 5′ CS region (e.g., as listed in Table 3). In some embodiments, the nucleic acid construct comprises a nucleic acid sequence having at least 75% (e.g., at least 75%, 80%, 85%, 90%, 95%, 96%, 97%, 98%, 99%, or 100%) sequence identity to a SLEV 3′ CS region (e.g., as listed in Table 3). In some embodiments, the nucleic acid construct comprises a nucleic acid sequence having at least 75% (e.g., at least 75%, 80%, 85%, 90%, 95%, 96%, 97%, 98%, 99%, or 100%) sequence identity to an SLEV NS3 sequence (e.g., as listed in Table 3), or the reverse complement thereof. In some embodiments, the nucleic acid construct comprises a nucleic acid sequence having at least 75% (e.g., at least 75%, 80%, 85%, 90%, 95%, 96%, 97%, 98%, 99%, or 100%) sequence identity to an SLEV NS5 sequence (e.g., as listed in Table 3), or the reverse complement thereof.

In some embodiments, the nucleic acid construct comprises a nucleic acid sequence having at least 75% (e.g., at least 75%, 80%, 85%, 90%, 95%, 96%, 97%, 98%, 99%, or 100%) sequence identity to an YFV 5′ UTR sequence (e.g., as listed in Table 4), or the reverse complement thereof. In some embodiments, the nucleic acid construct comprises a nucleic acid sequence having at least 75% (e.g., at least 75%, 80%, 85%, 90%, 95%, 96%, 97%, 98%, 99%, or 100%) sequence identity to an YFV 3′ UTR sequence (e.g., as listed in Table 4), or the reverse complement thereof. In some embodiments, the nucleic acid construct comprises a nucleic acid sequence having at least 75% (e.g., at least 75%, 80%, 85%, 90%, 95%, 96%, 97%, 98%, 99%, or 100%) sequence identity to a truncated YFV 5′ UTR sequence (e.g., as listed in Table 4), or the reverse complement thereof. In some embodiments, the nucleic acid construct comprises a nucleic acid sequence having at least 75% (e.g., at least 75%, 80%, 85%, 90%, 95%, 96%, 97%, 98%, 99%, or 100%) sequence identity to a truncated YFV 3′ UTR sequence (e.g., as listed in Table 4), or the reverse complement thereof. In some embodiments, the nucleic acid construct comprises a nucleic acid sequence having at least 75% (e.g., at least 75%, 80%, 85%, 90%, 95%, 96%, 97%, 98%, 99%, or 100%) sequence identity to a YFV 5′ CS region (e.g., as listed in Table 4). In some embodiments, the nucleic acid construct comprises a nucleic acid sequence having at least 75% (e.g., at least 75%, 80%, 85%, 90%, 95%, 96%, 97%, 98%, 99%, or 100%) sequence identity to a YFV 3′ CS region (e.g., as listed in Table 4). In some embodiments, the nucleic acid construct comprises a nucleic acid sequence having at least 75% (e.g., at least 75%, 80%, 85%, 90%, 95%, 96%, 97%, 98%, 99%, or 100%) sequence identity to an YFV NS5 sequence (e.g., as listed in Table 4), or the reverse complement thereof.

In some embodiments, the nucleic acid construct comprises a nucleic acid sequence having at least 75% (e.g., at least 75%, 80%, 85%, 90%, 95%, 96%, 97%, 98%, 99%, or 100%) sequence identity to an MVEV 5′ UTR sequence (e.g., as listed in Table 5), or the reverse complement thereof. In some embodiments, the nucleic acid construct comprises a nucleic acid sequence having at least 75% (e.g., at least 75%, 80%, 85%, 90%, 95%, 96%, 97%, 98%, 99%, or 100%) sequence identity to an MVEV 3′ UTR sequence (e.g., as listed in Table 5), or the reverse complement thereof. In some embodiments, the nucleic acid construct comprises a nucleic acid sequence having at least 75% (e.g., at least 75%, 80%, 85%, 90%, 95%, 96%, 97%, 98%, 99%, or 10%) sequence identity to a truncated MVEV in UTR sequence (e.g., as listed in Table 5), or the reverse complement thereof. In some embodiments, the nucleic acid construct comprises a nucleic acid sequence having at least 75% (e.g., at least 75%, 80%, 85%, 90%, 95%, 96%, 97%, 98%, 99%, or 100%) sequence identity to a truncated MVEV 3′ UTR sequence (e.g., as listed in Table 5), or the reverse complement thereof. In some embodiments, the nucleic acid construct comprises a nucleic acid sequence having at least 75% (e.g., at least 75%, 80%, 85%, 90%, 95%, 96%, 97%, 98%, 99%, or 100%) sequence identity to a MVEV 5′ CS region (e.g., as listed in Table 5). In some embodiments, the nucleic acid construct comprises a nucleic acid sequence having at least 75% (e.g., at least 75%, 80%, 85%, 90%, 95%, 96%, 97%, 98%, 99%, or 100%) sequence identity to a MVEV 3′ CS region (e.g., as listed in Table 5). In some embodiments, the nucleic acid construct comprises a nucleic acid sequence having at least 75% (e.g., at least 75%, 80%, 85%, 90%, 95%, 96%, 97%, 98%, 99%, or 100%) sequence identity to an MVEV NS5 sequence (e.g., as listed in Table 5), or the reverse complement thereof.

TABLE 2
Exemplary West Nile Virus (WNV) Sequences

| Description | Sequence | SEQ ID NO: |
|---|---|---|
| Full Length West | TTGVYRIMTRGLLGSYQAGAGVMVEGVFHTL | 17 |
| Nile Virus NS3 | WHTTKGAALMSGEGRLDPYWGSVICEDRLCYG | |
| (helicase) amino | GPWKLQHKWNGHDEVQMIVVEPGKNVKNVQ | |
| acid sequence | TKPGVFKTPEGEIGAVTLDYPTGTSGSPIVDKNG | |
| (Accession No. | DVIGLYGNGVIMPNGSYISAIVQ | |
| ABR19639.1: | | |
| 1524-1672) | | |
| Full Length West | EEDVNLGSGTRAVGKPLLNSDTSKIKNRIERLR | 18 |
| Nile Virus NS5 | REYSSTWHHDENHPYRTWNYHGSYDVRPTGS | |
| (RdRP) amino acid | ASSLVNGVVRLLSKPWDITFNVTTMAMTLYTIP | |
| sequence | FCQQRVFKEKVDTKAPEPPEGVKYVLNETTNW | |
| (Accession No. | LWAFLAREKRPRMCSREEFIRKVNSNAALGAM | |
| ACV90471.1: | FEEQNQWRSAREAVEDPKFWEMVDEEREAHL | |
| 2783-3433) | RGECHTCIYNMMGKREKKPGEFGKAKGSRAIW | |
| | FMWLGARFLEFEALGFLNEDHWLGRKNSGGG | |
| | VEGLGLQKLGYILREVGTRPGGKIYADDTAGW | |
| | DTRITRADLENEAKVLELLDGEHRRLARAIIELT | |
| | YRHKVVKVMRPAADGRTVMDVISREDQRGSG | |
| | QVVTYALNTFTNLAVQLVRMMEGEGVVGPDD | |
| | VEKLTKGKGPKVRTWLFENGEERLSRMAVSGD | |
| | DCVVKPLDDRFATSLHFLNAMSKVRKDIQEWK | |
| | PSTGWYDWQQVPFCSNHFTELIMKDGRTLVVP | |
| | CRGQDELVGRARISPGAGWNVRDTACLAKSYA | |
| | QMWLLLYFHRRDLRLMANAICSAVPVNWVPT | |
| | GRTTWSIHAGGEWMTTEDMLEVWNRVWIEEN | |
| | EWMEDKTPVEKWSDVPYSGKREDIWCGSLVG | |
| | TRARATWAENIQVAINQVRAIIGDEKYVDYMSS | |
| | LKRYEDTTLVEDTVL | |
| Truncated West | AGTAGTTCGCCTGTGTGAGCTGACAAACTTAG | 19 |
| Nile Virus (WNV) | TAGTGTTTGTGAGGATTAACAACAATTAACAC | |
| RdRP-responsive | AGTGCGAGCTGTTTCTTGGCACGAAGATCTCG | |
| 5′ UTR with CS | ATGTCTAAGAAACCAGGAGGGCCCGGTAAAA | |
| (truncated to | ACCGGGCTGTCAATATGCTA | |
| exclude | | |
| nucleotides | | |
| downstream from | | |
| the CS; *CS | | |
| underlined; | | |
| Accession No. | | |
| M12294.2: 1-147) | | |
| Truncated West | AGTAGTTCGCCTGTGTGAGCTGACAAACTTAG | 20 |
| Nile Virus RdRP- | TAGTGTTTGTGAGGATTAACAACAATTAACAC | |
| responsive 5′ UTR | AGTGCGAGCTGTTTCTTGGCACGAAGATCTCG | |
| (truncated to | ATGTCTAAGAAACCAGGAGGGCCCGGTAAAA | |
| exclude | ACCGGGCT | |
| nucleotides | | |
| beginning at the | | |
| CS) | | |
| Truncated West | AGCATATTGACACCTGGGATAGACTAGGGGA | 21 |
| Nile Virus RdRP- | TCTTCTGCTCTGCACAACCAGCCACACGGCAC | |
| responsive 3′ UTR | AGTGCGCCGACATAGGTGGCTGGTGGTGCTA | |
| with CS (truncated | GAACACAGGATCT | |
| to exclude | | |
| nucleotides | | |
| upstream from the | | |
| CS; * CS | | |
| underlined; | | |
| Accession No. | | |
| M12294.2: | | |
| 10856-10962) | | |
| Truncated West | ACCTGGGATAGACTAGGGGATCTTCTGCTCTG | 22 |
| Nile Virus RdRP- | CACAACCAGCCACACGGCACAGTGCGCCGAC | |
| responsive 3′ UTR | ATAGGTGGCTGGTGGTGCTAGAACACAGGAT | |
| (truncated to | CT | |
| exclude | | |
| nucleotides | | |
| beginning at the | | |
| CS) | | |

TABLE 3
Exemplary Saint Louis Encephalitis Virus (SLEV) Sequences

| Description | Sequence | SEQ ID NO: |
|---|---|---|
| Full Length Saint: | GGALWDVPSPKVYPKCETKPGIYRIMTRGILGT | 23 |
| Louis Encephalitis | FQAGVGVMHEGVFHTMWHATEGAVLRNGEG | |
| Virus NS3 | RLDPYAGDVRNDLISYGGPWKLSATWDGTEEV | |
| (helicase) | QMIAVAPGICPAINVQTTPGVEKTPLGTIGAVTL | |
| (Accession No. | DFPKGTSGSPIINKKGEIIGLYGNGVLIGQGEYVS | |
| YP_001008348.1.: | GIIQGERTEEPIPDAYNEEMLRKRKLTVLELHPG | |
| 1500-2117) | AGKTRKVLPQIIKDCIQKRLRTAVLAPTRVVAC | |
| | EIAEALKGLPIRYLTPAVRNEHQGNEIVDVMCH | |
| | ATLTQKLLTPTRVPNYQVYIMDEAHFIDPASIAA | |
| | RGYISTKVELGEAAAIFMTATPPGTNDPFPDSNS | |
| | PILDVEAQVPDKAWSTGYEWITNFTGRTVWFV | |
| | PSVKSGNEIAICLQKAGKRVIQLNRKSFDTEYPK | |
| | TKNNEWDFVVTTDISEMGANFGAHRVIDSRKC | |
| | VKPVILEDDDRVILNGPMAITSASAAQRRGRIGR | |
| | NPSQIGDEYHYGGATNEDDHDLANWTEAKILL | |
| | DNIYLPNGLVAQMYCPERDKVFTMDGEFRLRG | |
| | EERKNFVELMRNGDLPVWLAYKVASNGHSYQ | |
| | DRSWCFTGQTNNTILEDNNEVEVFTKTGDRKIL | |
| | RPKWMDARVCCDYQALKSFKEFAAGKR | |
| Full Length Saint | EEDVNLGSGTRSVGKLTEKPDPRKVGERIRRLR | 24 |
| Louis Encephalitis | EEYQQTWTYDHNNPYRTWNYHGSYEVKPTGS | |
| Virus NS5 (RdRP) | ASSMVNGVVRLLSKPWDMITNVTTMAMTDTT | |
| (Accession No. | PFGQQRVFKEKVDTKAPEPPLGVAQIMDVTTD | |
| YP_001008348.1: | WLWDFVAREKKPRVCTPEEFKAKVNSHAALG | |
| 2779-341.8) | AMFEEQNQWSSAREAVEDPKFWEMVDEEREA | |
| | HLKGECHTCIYNMMGKREKKTGEFGKAKGSR | |
| | AIWYMWLGARFLEFEALGFLNEDHWMSRENS | |
| | YGGVEGKGLQKLGYIWEISQIPGGKMYADDT | |
| | AGWDTRITKEDLKNEAKITKRMEERHRKLAEAI | |
| | IDLTYRHKVVKVMRPGPDGKTYMDVISREDQR | |
| | GSGQVVTYALNTFTNLAVQLIRCMEAEGVVDE | |
| | DDITRVRLGRLAKAVEWLRKNGPERLSRMAVS | |
| | GDDCVVKPIDDRFATALHFLNNMSKIRKDIQEW | |
| | KPSTGWHNWQEVPFCSHHFNELMLKDGRTIVV | |
| | PCRSQDELIGRARISPGAGWNVKETACLSKSYA | |
| | QMWLLMYFHRRDLRMMANAICSAVPVNWVPT | |
| | GRTTWSIHGKGEWMTTEDMLSVWNRVWIEEN | |
| | EYMIKDKTPLAAWNDIPYLGKREDIWCGSLIGT | |
| | RTRATWAENIYAPIMQIRNLIGEEEYRDYMVAQ | |
| | NRF | |
| Truncated Saint | AGATGTTCGCGTCGGTGAGCGGAGAGGAAAC | 25 |
| Louis Encephalitis | AGATTTCCTTTTGGAGGATAATAACTTTAACT | |
| Virus (SLEV) | TGACTGCGAACAGTTTTTTAGCAGGGAATTAC | |
| RdRP-responsive | CCAATGTCTAAAAAACCAGGAAAACCCGGTA | |
| 5′ UTR with CS | GAAACCGGGTTGTCAATATGCT | |
| (truncated to | | |
| exclude | | |
| nucleotides | | |
| beginning at the | | |
| CS; *CS | | |
| underlined; | | |
| Accession No. | | |
| NC_007580.2: | | |
| 1-130) | | |
| Truncated Saint | AGATGTTCGCGTCGGTGAGCGGAGAGGAAAC | 26 |
| Louis Encephalitis | AGATTTCCTTTTTGGAGGATAATAACTTAACT | |
| Virus RdRP- | TGACTGCGAACAGTTTTTTAGCAGGGAATTAC | |
| responsive 5′ UTR | CCAATGTCTAAAAAACCAGGAAAACCCGGTA | |
| (truncated to | GAAACCGGGTT | |
| exclude | | |
| nucleotides | | |
| beginning at the | | |
| CS) | | |
| Truncated Saint | AGCATATTGACACCTGGAAAGACAGGAGATC | 27 |
| Louis Encephalitis | CCCTGCTTTTTCAACACCAGCCACAAGGCACA | |
| Virus RdRP- | GAGCGCCGTAAAGTGTGGCTGCTGGTGAAAA | |
| responsive 3′ UTR | AATCACAGGATCT | |
| with CS (truncated | | |
| to exclude | | |
| nucleotides | | |
| upstream from the | | |
| CS; * CS | | |
| underlined; | | |
| Accession No. | | |
| NC_007580.2: | | |
| 10389-10940) | | |
| Truncated Saint | ACCTGGAAAGACAGGAGATCCCCTGCTTTTTC | 28 |
| Louis Encephalitis | AACACCAGCCACAAGGCACAGAGCGCCGTAA | |
| Virus RdRP- | AGTGTGGCTGGTGGTGAAAAAATCACAGGAT | |
| responsive 3′ UTR | CT | |
| (truncated to | | |
| exclude | | |
| nucleotides | | |
| beginning at the | | |
| CS) | | |

TABLE 4
Exemplary Yellow Fever Virus (YFV) Sequences

| Description | Sequence | SEQ ID NO: |
|---|---|---|
| Full Length | TLEADVILPIGTRSVETDKGPLDRAAIFERVERIK | 29 |
| Yellow Fever | SEYTATWFHDNDNPYRTWHYCGSYVTRTSGSA | |
| Virus NS5 (RdRP) | ASMINGVIKILTYPWDRIEEVTRMAMTDTTPFG | |
| amino acid | QQRVFKEKVDTRAKDPPAGTRKIMKVVNRWLF | |
| sequence | RHLAREKNPRLCTKEEFIAKVRSHAAIGAFLEE | |
| (Accession No. | QEQWKTANEAVQDPKFWELVDEERRLHQQGR | |
| AIZ07887.1: | CRTCVYNMMGKREKKLASEFGKAKGSRAIWYM | |
| 2758-3406) | WLGARYLEFEALGTLNEDHWASRENSGGGVEG | |
| | IGLQYLGYVIRDLATLEGGGFYADDTAGWDTRI | |
| | TEADLDDEQEILNYMSPHHRKLALAVMEMTYK | |
| | NKVVKVLRPAPGGKAYMDVISRRDQRGSGQV | |
| | VTYALNTITNLKVQLIRMAEAEMVIHHQHVQD | |
| | CDDTVLTKLEAWLTEHGCDRLKRMAVSGDDC | |
| | VVRPIDDRFGLALSHLNAMSKVRKDISEWQPSK | |
| | GWDDWENVPFCSHHFHELQLKDGRRIVVPCRD | |
| | QDELVGRGRVSPGNGWMIKETACLSKAYANM | |
| | WSLMYFHKRDMRLLSLAVSSAVPTSWVPQGRT | |
| | TWSVHGKGEWMTTEDMLEVWNRVWITNNPH | |
| | MQDKTTVKEWRDIPYLTKRQDKLCGSLIGMTN | |
| | RATWASHIHLVIHRIRTLIGKERYTDYLTVMDR | |
| | YSVDADLQ | |
| Truncated Yellow | AGTAAATCCTGTGTGCTAATTGAGGTGCATTG | 30 |
| Fever Virus (YFV) | GTCTGCAAATCGAGTTGCTAGGCAATAAACA | |
| RdRP-responsive | CATTTGGATTAATTTTAATCGTTCGTTGAGCG | |
| 5′ UTR with CS | ATTAGCAGAGAACTGACCAGAACATGTCTGG | |
| (truncated to | TCGTAAAGCTCAGGGAAAAACCCTGGGCGTC | |
| exclude | AATATGGT | |
| nucleotides | | |
| downstream of the | | |
| cyclization | | |
| sequence (CS); | | |
| *CS underlined: | | |
| Accession No. | | |
| NC_002031.1: | | |
| 1-165) | | |
| Truncated Yellow | AGTAAATCCTGTGTGCTAATTGAGGTGCATTG | 31 |
| Fever Virus RdRP- | GTCTGCAAATCGAGTTGCTAGGCAATAAACA | |
| responsive 5′ UTR | CATTTGGATTAATTTTAATCGTTCGTTGAGCG | |
| (truncated to | ATTAGCAGAGAACTGACCAGAACATGTCTGG | |
| exclude | TCGTAAAGCTCAGGGAAAAA | |
| nucleotides | | |
| beginning at the | | |
| CS) | | |
| Truncated Yellow | ACCATATTGACGCCAGGGAAAGACCGGAGTG | 32 |
| Fever Virus RdRP- | GTTCTCTGCTITTCCTCCAGAGGTCTGTGAGC | |
| responsive 3′ UTR | ACAGTTTGCTCAAGAATAAGCAGACCTTTGG | |
| with CS (truncated | ATGACAAACACAAAACCACT | |
| to exclude | | |
| nucleotides | | |
| upstream from the | | |
| CS; *CS | | |
| underlined; | | |
| Accession No. | | |
| NC_002031.1: | | |
| 10749-10862) | | |
| Truncated Yellow | AAAGACCGGAGTGGTTCTCTGCTTTTCCTCCA | 33 |
| Fever Virus RdRP- | GAGGTCTGTGAGCACAGTTTGCTCAAGAATA | |
| responsive 3′ UTR | AGCAGACCTTTGGATGACAAACACAAAACCA | |
| (truncated to | CT | |
| exclude | | |
| nucleotides | | |
| beginning at the | | |
| CS) | | |

TABLE 5
Exemplary Murray Valley Encephalitis Virus (MVEV) Sequences

| Description | Sequence | SEQ ID NO: |
|---|---|---|
| Full length Murray | EEDVNLGSGTRAVGKGVQHTDYKRIKSRIEKLK | 34 |
| Valley Encephalitis | EEYAATWHTDDNHPYRTWTYHGSYEVKPSGS | |
| Virus NS5 (RdRP) | ASTLYNGVVRLLSKPWDAITGVTTMAMTDTTP | |
| amino acid | FGQQRVFKEKVDTKAPEPPQGVKTVMDETTN | |
| sequence | WLWAYLARNKKARLCTREEFVKKVNSHAALG | |
| (Accession No. | AMFEEQNQWKNAREAVEDPKFWEMVDEEREC | |
| NP_051124.1: | HLRGECRTCIYNMMGKREKKPGEFGKAKGSRA | |
| 2784-3423) | IWFMWLGARFLEFEALGFLMHDHWMSRENSGG | |
| | GVEGAGIQKLGYILRDVAQKPGGKIYADDTAG | |
| | WDTRITQADLENEAKVLELMEGEQRTLARAIIE | |
| | LTYRHKVVKVMRPAAGGKTVMDVISREDQRG | |
| | SGQVVTYALNTFTNIAVQLVRLMEAEAVIGPDD | |
| | IESIERKKKFAVRTWLFENAEERVQRMAVSGDD | |
| | CVVKPLDDRFSTALLIFLNAMSKVRKDIQEWKP | |
| | SQGWYDWQQVPFCSNHFQEVIMKDGRTLVVP | |
| | CRGQDEUGRARISPGSGWNVRDTACLAKAYA | |
| | QMWILNLYFFIRRDLRLMANAICSSVPVDWVPT | |
| | GRTTWSIHGKGEWMTTEDMLSVWNRVWILEN | |
| | EWMEDKTTVSDWTEVPYVGKREDIWCGSLIGT | |
| | RTRATWAENIYAAINQVRSVICiKEKYVDYVQS | |
| | LRRY | |
| Truncated Murray | AGACGTTCATCTGCGTGAGCTTCCGATCTCAG | 35 |
| Valley Encephalitis | TATTGTTTGGAAGCATCATTGATTAACGCGGT | |
| Virus (MVEV) | TTGAACAGTTTTTTGGAGCTTTTCiATTTCAAA | |
| RdRP-responsive | TGTCTAAAAAACCAGGAGGACCCGGGAAGCC | |
| 5′ UTR with CS | CCGGGTCGTCAATATGCTA | |
| (truncated to | | |
| exclude | | |
| nucleotides | | |
| downstream of the | | |
| cyclization | | |
| sequence (CS); | | |
| *CS underlined; | | |
| Accession No. | | |
| NC_00943.1: | | |
| 1-146) | | |
| Truncated Murray | AGACGTTCATCTGCGTGAGCTTCCGATCTCAG | 36 |
| Valley Encephalitis | TATTGTTTGGAAGGATCATTGATTAACGCGGT | |
| Virus RdRP- | TTGAACAGTTTTTTGGAGCTTTTGATTTCAAA | |
| responsive 5′ UTR | TGTCTAAAAAACCAGGAGGACCCGGGAAGCC | |
| (truncated to | CCGGGTC | |
| exclude | | |
| nucleotides | | |
| beginning at the | | |
| CS) | | |
| Truncated Murray | AGCATATTGACACCTGGGAAAAGACTAGGAG | 37 |
| Valley Encephalitis | ATCTTCTGCTCTATTCCAACATCAGTCACAAG | |
| Virus RdRP- | GCACCGAGCGCCGAACACTGTGACTGATGGG | |
| responsive 3′ UTR | GGAGAAGACCACAGGATCT | |
| with CS (truncated | | |
| to exclude | | |
| nucleotides | | |
| upstream of the | | |
| CS; *CS | | |
| underlined; | | |
| Accession No. | | |
| NC_000943.1: | | |
| 10902-11014) | | |
| Truncated Murray | ACCTGGGAAAAGACTAGGAGATCTTCTGCTC | 38 |
| Valley Encephalitis | TATTCCAACATCAGTCACAAGGCACCGAGCG | |
| Virus RdRP- | CCGAACACTGTGACTGATGGGGGAGAAGACC | |
| responsive 3′ UTR | ACAGGATCT | |
| (truncated to | | |
| exclude | | |
| nucleotides | | |
| beginning at the | | |
| CS) | | |

### Exemplary Nucleic Acid Constructs

The present disclosure provides amplifiable nucleic acid constructs suitable for expression of a gene of interest. The nucleic acid constructs generally comprise RdRP-responsive 5′ UTR and/or RdRP-responsive 3′ UTR elements flanking an open reading frame comprising the gene of interest, e.g., as described herein. The nucleic acid constructs may include one or more such open reading frames (e.g., encoding one or more genes of interest and/or an RdRP). In some embodiments, the nucleic acid construct does not comprise an RdRP compatible with the RdRP-responsive UTRs comprised in the nucleic acid construct. In some embodiments, the nucleic acid construct encodes an RdRP compatible with the RdRP-responsive UTR(s) comprised in the nucleic acid construct. In some embodiments, when there are more than one (e.g. two or more) genes of interest, the genes of interest may be comprised in a single nucleic acid construct or in different nucleic acid constructs. When both a gene of interest and an RdRP are encoded by the same nucleic acid construct, there are multiple possible sub-strategies useful for co-expression of the gene of interest and the RdRP. In some embodiments, the nucleic acid construct comprises a separate promoter for the gene of interest and the RdRP-encoding gene. In some embodiments, the nucleic acid construct comprises an IRES positioned between the gene of interest and the RdRP-encoding gene. In some embodiments, the nucleic acid construct comprises a protease cleavage sequence and/or a self-cleaving sequence. e.g., a 2A sequence, positioned between the gene of interest and the RdRP-encoding gene, e.g., as described herein).

In some embodiments, a nucleic acid construct described herein comprises a linker, e.g., having a length of 1-10, 1-20, 1-50, or 1-100 nucleotides. In some embodiments, the linker is situated between the terminal end of a RdRP-responsive 5′ UTR and a nucleic acid encoding an exogenous protein. In some embodiments, the linker is situated between the terminal end of a nucleic acid encoding an exogenous protein and a RdRP-responsive 3′ UTR. In some embodiments, the linker is situated between the terminal end of a 5′ CS region and a nucleic acid encoding an exogenous protein. In some embodiments, the linker is situated between the terminal end of a nucleic acid encoding an exogenous protein and a 3′ CS region.

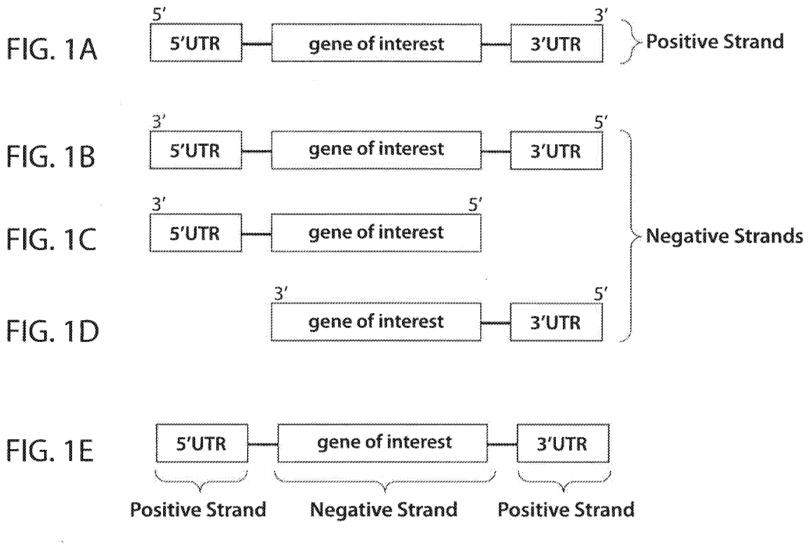

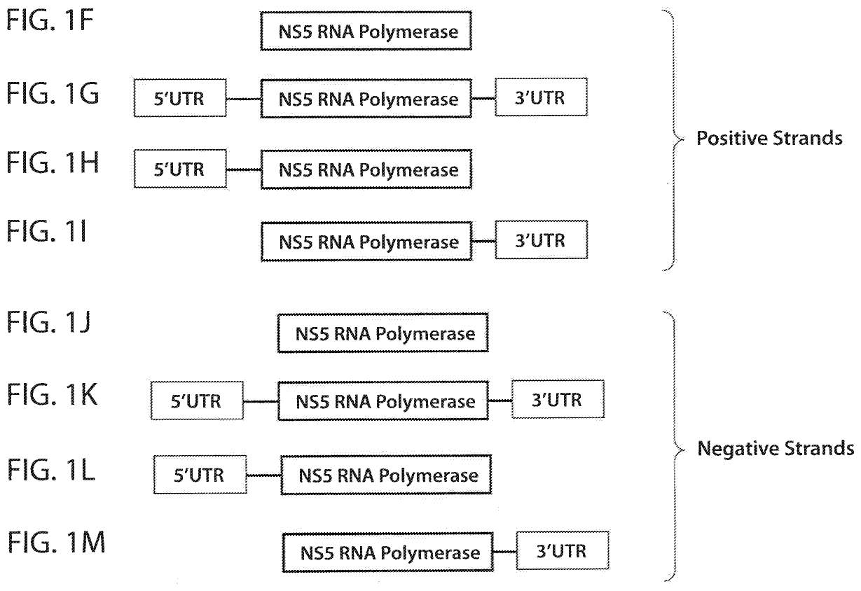

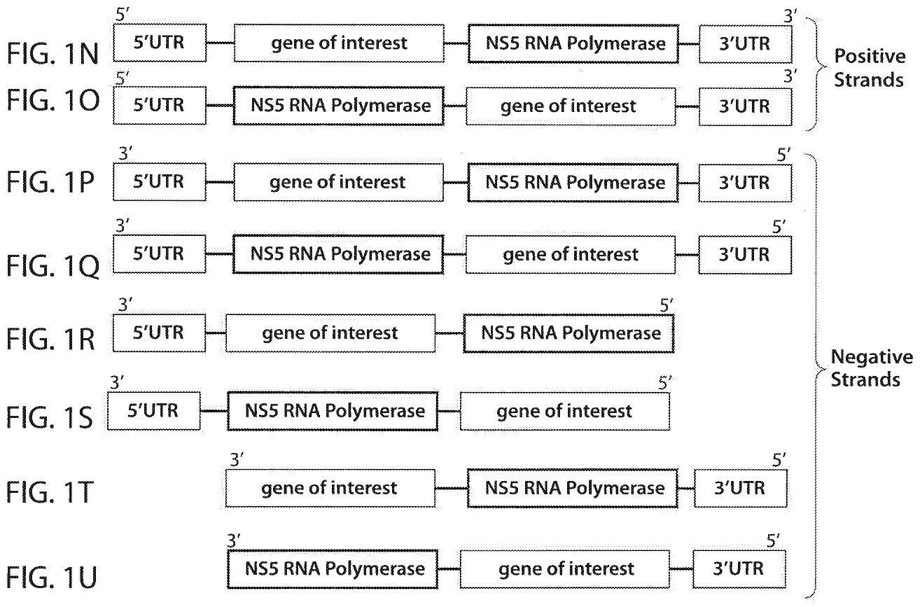

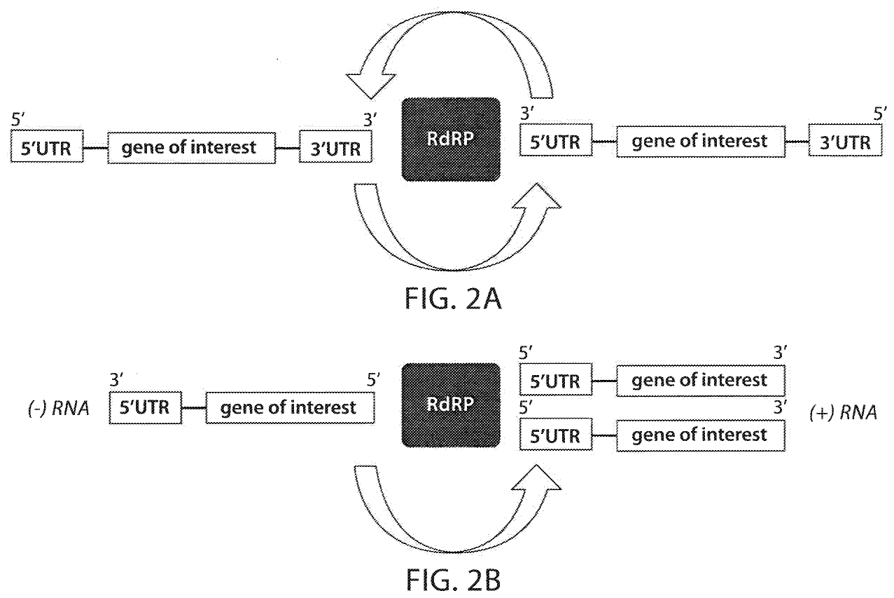

The nucleic acid constructs described herein can, in some instances, be RNA constructs. Non-limiting examples of arrangements for RNA constructs are provided below and in FIGS. 1A-2B.

In some embodiments, the RNA construct is a positive strand comprising, from 5′ to 3′, an RdRP-responsive 5′ UTR, an exogenous nucleic acid sequence, and an RdRP-responsive 3′ UTR (see, e.g., FIG. 1A). In some embodiments, the RNA construct is a positive strand comprising, from 5′ to 3′, an RdRP-responsive 3′ UTR, an exogenous nucleic acid sequence, and an RdRP-responsive 5′ UTR. In some embodiments, the RNA construct is a positive strand comprising, from 5′ to 3′, an RdRP-responsive 5′ UTR and an exogenous nucleic acid sequence. In some embodiments, the RNA construct is a positive strand comprising, from 5′ to 3′, an exogenous nucleic acid sequence and an RdRP-responsive 5′ UTR.

In some embodiments, the RNA construct is a negative strand comprising, from 5′ to 3′, an RdRP-responsive 5′ UTR, an exogenous nucleic acid sequence, and an RdRP-responsive 3′ UTR. In some embodiments, the RNA construct is a negative strand comprising, from 5′ to 3′, an RdRP-responsive 3′ UTR, an exogenous nucleic acid sequence, and an RdRP-responsive 5′ UTR (see, e.g., FIG. 1B). In some embodiments, the RNA construct is a negative strand comprising, from 5′ to 3′, an RdRP-responsive 5′ UTR and an exogenous nucleic acid sequence. In some embodiments, the RNA construct is a negative strand comprising, from 5′ to 3′, an exogenous nucleic acid sequence and an RdRP-responsive 5′ UTR (see. e.g., FIG. 1C). In some embodiments, the negative strand RNA construct lacks a functional RdRP-responsive 3′ UTR, e.g., lacks an RdRP-responsive 3′ UTR. In some embodiments, the negative strand RNA construct comprises a 3′ UTR that does not bind an RdRP, e.g., a mammalian 3′ UTR. In some embodiments, the negative strand RNA construct can undergo a round of amplification (e.g., mediated by an RdRP) to produce a positive strand RNA, but the resultant positive strand RNA cannot then undergo amplification to produce a copy of the original negative strand RNA (e.g., due to the presence of the RdRP-responsive 5′ UTR and the absence of a functional RdRP-responsive 3′ UTR in the original negative strand RNA).

In some embodiments, the RNA construct comprises, from 5′ to 3′, a positive strand RdRP-responsive 5′ UTR, a negative strand exogenous nucleic acid sequence, and a positive strand RdRP-responsive 3′ UTR. In some embodiments, the RNA construct comprises, from 5′ to 3′, a negative strand RdRP-responsive 5′ UTR, a positive strand exogenous nucleic acid sequence, and a negative strand RdRP-responsive 3′ UTR. In some embodiments, the RNA construct comprises, from 5′ to 3′, a positive strand RdRP-responsive 5′ UTR and a negative strand exogenous nucleic acid sequence.

In some embodiments, the RNA construct comprises one or more additional elements. In certain embodiments, the RNA construct comprises a 5′ cap (e.g., a m7GpppAMP cap). In certain embodiments, the RNA construct comprises a poly-A tail. In some embodiments, the RNA construct comprises a modified nucleotide and/or a modified cap. In some embodiments, the exogenous mRNA comprises one or more chemically modified nucleotides of Table 1 of PCT Publication No. WO/2018/009838 (incorporated by reference herein), one or more chemical backbone modifications of Table 2 of PCT Publication No. WO/2018/009838u and/or one or more chemically modified caps of Table 3 of PCT Publication No. WO/2018/009838. In some embodiments, the exogenous mRNA comprises one or more chemically modified nucleotides of Table 6. In some embodiments, the exogenous mRNA comprises one or more chemical backbone modifications of Table 7. In some embodiments, the exogenous mRNA comprises one or more chemically modified caps of Table 8.

TABLE 6
Exemplary modified nucleotides

| | |
|---|---|
| 5-aza-uridine | N2-methyl-6-thio-guanosine |
| 2-thio-5-aza-midine | N2,N2-dimethyl-6-thio-guanosine |
| 2-thiouridine | pyridin-4-one ribonucleoside |
| 4-thio-pseudouridine | 2-thio-5-aza-uridine |
| 2-thio-pseudouridine | 2-thiomidine |
| 5-hydroxyuridine | 4-thio-pseudomidine |
| 3-methyluridine | 2-thio-pseudowidine |
| 5-carboxymethyl-uridine | 3-methylmidine |
| 1-carboxymethyl-pseudouridine | 1-propynyl-pseudomidine |
| 5-propynyl-uridine | 1-methyl-1-deaza-pseudomidine |
| 1-propynyl-pseudouridine | 2-thio-1-methyl-1-deaza-pseudouridine |
| 5-taurinomethyluridine | 4-methoxy-pseudomidine |
| 1-taurinomethyl-pseudouridine | 5′-O-(1-Thiophosphate)-Adenosine |
| 5-taurinomethyl-2-thio-uridine | 5′-O-(1-Thiophosphate)-Cytidine |
| 1-taurinomethyl-4-thio-uridine | 5′-O-(1-thiophosphate)-Guanosine |
| 5-methyl-uridine | 5′-O-(1-Thiophophate)-Uridine |
| 1-methyl-pseudouridine | 5′-O-(1-Thiophosphate)-Pseudouridine |
| 4-thio-1-methyl-pseudouridine | 2′-O-methyl-Adenosine |
| 2-thio-1-methyl-pseudouridine | 2′-O-methyl-Cytidine |
| 1-methyl-1-deaza-pseudouridine | 2′-O-methyl-Guanosine |
| 2-thio-1-methyl-1-deaza-pseudomidine | 2′-O-methyl-Uridine |
| dihydrouridine | 2′-O-methyl-Pseudouridine |
| dihydropseudouridine | 2′-O-methyl-Inosine |
| 2-thio-dihydromidine | 2-methyladenosine |
| 2-thio-dihydropseudouridine | 2-methylthio-N6-methyladenosine |
| 2-methoxyuridine | 2-methylthio-N6 isopentenyladenosine |
| 2-methoxy-4-thio-uridine | 2-methylthio-N6-(cis- |
| 4-methoxy-pseudouridine | hydroxyisopentenyl)adenosine |
| 4-methoxy-2-thio-pseudouridine | N6-methyl-N6-threonylcarbamoyladenosine |
| 5-aza-cytidine | N6-hydroxynorvalylcarbamoyladenosine |
| pseudoisocytidine | 2-methylthio-N6-hydroxynorvalyl |
| 3-methyl-cytidine | carbamoyladenosine |
| N4-acetylcytidine | 2′-O-ribosyladenosine (phosphate) |
| 5-formylcytidine | 1,2′-O-dimethylinosine |
| N4-methylcytidine | 5,2′-O-dimethylcytidine |
| 5-hydroxvmethylcytidine | N4-acetyl-2′-O-methylcytidine |
| 1-methyl-pseudoisocytidine | Lysidine |
| pyrrolo-cytidine | 7-methylguanosine |
| pyrrolo-pseudoisocytidine | N2,2′-O-dimethylguanosine |
| 2-thio-cytidine | N2,N2,2′-O-trimethylguanosine |
| 2-thio-5-methyl-cytidine | 2′-O-ribosylguanosine (phosphate) |
| 4-thio-pseudoisocytidine | Wybutosine |
| 4-thio-1-methyl-pseudoisocytidine | Peroxywybutosine |
| 4-thio-1-methyl-1-deaza-pseudoisocytidine | Hydroxywybutosine |
| 1-methyl-1-deaza-pseudoisocytidine | undermodified hydroxywybutosine |
| zebularine | methylwyosine |
| 5-aza-zebularine | queuosine |
| 5-methyl-zebularine | epoxyqueuosine |
| 5-aza-2-thio-zebularine | galactosyl-queuosine |
| 2-thio-zebularine | mannosyl-queuosine |
| 2-methoxy-cytidine | 7-cyano-7-deazaguanosine |
| 2-methoxy-5-methyl-cytidine | 7-aminomethyl-7-deazaguanosine |
| 4-methoxy-pseudoisocytidine | archaeosine |
| 4-methoxy-1-methyl-pseudoisocytidine | 5,2′-O-dimethyluridine |
| 2-aminopurine | 4-thiouridine |
| 2,6-diaminopurine | 5-methyl-2-thiouridine |
| 7-deaza-adenine | 2-thio-2′-O-methyluridine |
| 7-deaza-8-aza-adenine | 3-(3-amino-3-carboxypropyl)uridine |
| 7-deaza-2-aminopurine | 5-methoxyuridine |
| 7-deaza-8-aza-2-aminopurine | uridine 5-oxyacetic acid |
| 7-deaza-2,6-diaminopurine | uridine 5-oxyacetic acid methyl ester |
| 7-deaza-8-aza-2,6-diaminopurine | 5-(carboxyhydroxymethyl)uridine) |
| 1-methyladenosine | 5-(carboxyhydroxymethyl)uridine methyl ester |
| N6-isopentenyladenosine | 5-methoxycarbonylmethyluridine |
| N6-(cis-hydroxyisopentenyl)adenosine | 5-methoxycarbonylmethyl-2′-O-methyluridine |
| 2-methylthio-N6-(cis-hydroxyisopentenyl) | 5-methoxycarbonylmethyl-2-thiouridine |
| adenosine | 5-aminomethyl-2-thiouridine |
| N6-glycinylcarbamoyladenosine | 5-methylaminomethyluridine |
| N6-threonylcarbamoyladenosine | 5-methylaminomethyl-2-thiouridine |
| 2-methylthio-N6-threonyl | 5-methylaminomethyl-2-selenouridine |
| carbamoyladenosine | 5-carbamoylmethyluridine |
| N6,N6-dimethyladenosine | 5-carbamoylmethyl-2′-O-methyluridine |
| 7-methyladenine | 5-carboxymethylaminomethyluridine |
| 2-methylthio-adenine | 5-carboxymethylaminomethyl-2′-O- |
| 2-methoxy-adenine | methyluridine |
| inosine | 5-carboxymethylaminomethyl-2-thiouridine |
| 1-methyl-inosine | N4,2′-O-dimethylcytidine |
| wyosine | 5-carboxymethyluridine |
| wybutosine | N6,2′-O-dimethyladenosine |
| 7-deaza-guanosine | N,N6,O-2′-trimethyladenosine |
| 7-deaza-8-aza-guanosine | N2,7-dimethylguanosine |
| 6-thio-guanosine | N2,N2,7-trimethylguanosine |
| 6-thio-7-deaza-guanosine | 3,2′-O-dimethyluridine |
| 6-thio-7-deaza-8-aza-guanosine | 5-methyldihydrouridine |
| 7-methyl-guanosine | 5-formyl-2′-O-methylcytidine |
| 6-thio-7-methyl-guanosine | 1,2′-O-dimethylguanosine |
| 7-methylinosine | 4-demethylwyosine |
| 6-methoxy-guanosine | Isowyosine |
| 1-methylguanosine | N6-acetyladenosine |
| N2-methylguanosine | |
| N2,N2-dimethylguanosine | |
| 8-oxo-guanosine | |
| 7-methyl-8-oxo-guanosine | |
| 1-methyl-6-thio-guanosine | |

TABLE 7
Exemplary backbone modifications

2′-O-Methyl backbone
Peptide Nucleic Acid (PNA) backbone
phosphorothioate backbone
morpholino backbone
carbamate backbone
siloxane backbone
sulfide backbone
sulfoxide backbone
sulfone backbone
formacetyl backbone
thioformacetyl backbone
methyleneformacetyl backbone
riboacetyl backbone
alkene containing backbone
sulfamate backbone
sulfonate backbone
sulfonamide backbone
methyleneimino backbone
methylenehydrazino backbone
amide backbone

TABLE 8
Exemplary modified caps

m7GpppA
m7GpppC
m2,7GpppG
m2,2,7GpppG
m7Gpppm7G
m7,2′OmeGpppG
m72′dGpppG
m7,3′OmeGpppG
m7,3′dGpppG
GppppG
m7GppppG
m7GppppA
m7GppppC
m2,7GppppG
m2,2,7GppppG
m7Gppppm7G
m7,2OmeGppppG
m72′dGppppG
m7,3′OmeGppppG
m7,3′dGppppG

In some embodiments, the RNA construct comprises one or more of ARCA: anti-reverse cap analog (m27.3′-OGP3G). GP3G (Unmethylated Cap Analog), m7GP3G (Monomethylated Cap Analog), m32.2.7GP3G (Trimethylated Cap Analog), m5CTP (5′-methyl-cytidine triphosphate), m6ATP (N6-methyl-adenosine-5′-triphosphate), s2UTP (2-thio-uridine triphosphate), and Ψ (pseudouridine triphosphate). In embodiments, the RNA construct comprises N6-methyladenosine. In embodiments, the RNA construct comprises pseudouridine.

In any of the embodiments described herein, e.g., embodiments in which a UTR does not provide an IRES sequence, an IRES sequence may be included, e.g., situated between the UTR and the coding region for the exogenous protein. In embodiments, the IRES sequence is situated upstream (e.g., immediately upstream) of the coding region (e.g., immediately upstream from the start codon of the gene encoding the exogenous protein. In some embodiments, the IRES sequence is situated within about 1, 2, 3, 4, 5, 6, 7, 8, 9, 10, 11, 12, 13, 14, 15, 16, 17, 18, 19, 20, 25, 30, 35, 40, 45, 50, 75, 100, 150, 200, or 250 nucleotides from the start codon of the gene encoding the exogenous protein. Multiple IRES sequences are known in the art, including but not limited to, IRESs derived from hepatitis C virus (HCV), murine stem cell virus (MSCV), and other IREs sequence described in U.S. Pat. No. 7,468,275, US Patent Publication Nos. US 2007/0048776 and US 2011/0124100 and International Patent Publication Nos. WO 2007/025008 and WO 2001/055369, each of which is herein incorporated by reference in its entirety. In some embodiments, the IRES is an MSCV-derived IRES that comprises or consists of the nucleic acid sequence provided in SEQ ID NO: 39 (below):

(SEQ ID NO: 39)
GCGGCCGCGCCCCTCTCCCTCCCCCCCCCCTAACGTTACTGGCCGAAGCC
GCTTGGAATAAGGCCGGTGTGCGTTTGTCTATATGTTATTTTCCACCATA
TTGCCGTCTTTTGGCAATGTGAGGGCCCGGAAACCTGGCCCTGTCTTCTT
GACGAGCATTCCTAGGGGTCTTTCCCCTCTCGCCAAAGGAATGCAAGGTC
TGTTGAATGTCGTGAAGGAAGCAGTTCCTCTGGAAGCTTCTTGAAGACAA
ACAACGTCTGTAGCGACCCTTTGGAGGCAGCGGAACCCCCCACCTGGCGA
CAGGTGCCTCTGCGGCCAAAAGCCACGTGTATAAGATACACCRGCAAAGG
CGGCACAACCCCAGTGCCACGTTGTGAGTTGGATAGTTGTGGAAAGAGTC
AAATGGCTCTCCTCAAGCGTATTCAACAAGGGGCTGAAGGATGCCCAGAA
GGTACCCCATTGTATGGGATCTGATCTGGGGCCTCGGTGCACATGCTTTA
CATGTGTTTAGTCGAGGTTAAAAAAACGTCTAGGCCCCCCGAACCACGGG
GACGTGGTTTTCCTTTGAAAAACACGATGATAATATGGCCA

In some embodiments, the RNA construct comprises a Kozak sequence. Kozak sequence refers to a short nucleotide sequence that facilitates the initial binding of mRNA to the small subunit of the ribosome and increases translation (see, e.g., Kozak (1986) Cell 44 (2): 283-92, and Kozak (1987) Nucleic Acids Res. 15(20): 8125-48). In some embodiments, the Kozak sequence comprises the consensus sequence (GCC)RCCATGG, where R is a purine (A or G) (SEQ ID NO: 39). Skilled artisans will readily understand that Kozak sequences generally overlap with the start codon of coding regions, and thus the coding region of a gene can be modified to include a functional Kosak sequence using methods known in the art.

RNA Constructs Comprising RdRP-Encoding Sequences

In some embodiments, the RNA construct further comprises a nucleic acid sequence encoding an RdRP that is compatible with the RdRP-responsive 5′ UTR and/or the RdRP-responsive 3′UTR of the construct (e.g., a nucleic acid sequence encoding a Dengue virus NS5 protein or a functional variant or fragment thereof). In some embodiments, the RdRP is a flavivirus RdRP (e.g., a Dengue virus RdRP), e.g., as described herein. In some embodiments, the RdRP is a non-flavivirus RdRP. Non-limiting examples of such RNA constructs are shown in FIGS. 1N-1U.

In some embodiments, the RNA construct comprises, in order, an RdRP-responsive 5′ UTR, an exogenous nucleic acid sequence, a nucleic acid sequence encoding an RdRP (e.g., a Dengue virus NS5 protein or a functional variant or fragment thereof), and an RdRP-responsive 3′ UTR. In some embodiments, the RNA construct comprises, in order, an RdRP-responsive 5′ UTR, a nucleic acid sequence encoding an RdRP (e.g., a Dengue virus NS5 protein or a functional variant or fragment thereof), an exogenous nucleic acid sequence, and an RdRP-responsive 3′ UTR. In certain embodiments, the order is 5′ to 3′. In other embodiments, the order is 3′ to 5′. In certain embodiments, the exogenous nucleic acid sequence is a positive strand sequence and the nucleic acid sequence encoding the RdRP is a positive strand sequence. In certain embodiments, the exogenous nucleic acid sequence is a positive strand sequence and the nucleic acid sequence encoding the RdRP is a negative strand sequence. In certain embodiments, the exogenous nucleic acid sequence is a negative strand sequence and the nucleic acid sequence encoding the RdRP is a positive strand sequence. In certain embodiments, the exogenous nucleic acid sequence is a negative strand sequence and the nucleic acid sequence encoding the RdRP is a negative strand sequence.

In embodiments, the RNA construct is a positive strand comprising, from 5′ to 3′, an RdRP-responsive 5′ UTR, an exogenous nucleic acid sequence, a nucleic acid sequence encoding an RdRP (e.g., a Dengue virus NS5 protein or a functional variant or fragment thereof), and an RdRP-responsive 3′ UTR. In embodiments, the RNA construct is a positive strand comprising, from 5′ to 3′, an RdRP-responsive 5′ UTR, a nucleic acid sequence encoding an RdRP (e.g., a Dengue virus NS5 protein or a functional variant or fragment thereof), an exogenous nucleic acid sequence, and an RdRP-responsive 3′ UTR. In embodiments, the RNA construct is a positive strand comprising, from 5′ to 3′, an RdRP-responsive 5′ UTR, an exogenous nucleic acid sequence, and a nucleic acid sequence encoding an RdRP (e.g., a Dengue virus NS5 protein or a functional variant or fragment thereof). In embodiments, the RNA construct is a positive strand comprising, from 5′ to 3′, an RdRP-responsive 5′ UTR, a nucleic acid sequence encoding an RdRP (e.g., a Dengue virus NS5 protein or a functional variant or fragment thereof), and an exogenous nucleic acid sequence. In embodiments, the RNA construct has the same orientation as any of the above constructs except that the positions of the Dengue virus 5′ UTR and the Dengue virus 3′ UTR are switched. In some negative control embodiments, the RNA construct is a positive strand comprising, from 5′ to 3′, a nucleic acid sequence encoding an RdRP, an exogenous nucleic acid sequence, and an RdRP-responsive 3′ UTR. In some negative control embodiments, the RNA construct is a positive strand comprising, from 5′ to 3′, a nucleic acid sequence encoding an exogenous nucleic acid sequence, an RdRP, and an RdRP-responsive 3′ UTR.

In embodiments, the RNA construct is a negative strand comprising, from 3′ to 5′, an RdRP-responsive 5′ UTR, an exogenous nucleic acid sequence, a nucleic acid sequence encoding an RdRP (e.g., a Dengue virus NS5 protein or a functional variant or fragment thereof), and an RdRP-responsive 3′ UTR. In embodiments, the RNA construct is a negative strand comprising, from 3′ to 5′, an RdRP-responsive 5′ UTR, a nucleic acid sequence encoding an RdRP (e.g., a Dengue virus NS5 protein or a functional variant or fragment thereof), an exogenous nucleic acid sequence, and an RdRP-responsive 3′ UTR. In embodiments, the RNA construct is a negative strand comprising, from 3′ to 5′, an RdRP-responsive 5′ UTR, an exogenous nucleic acid sequence, and a nucleic acid sequence encoding an RdRP (e.g., a Dengue virus NS5 protein or a functional variant or fragment thereof). In embodiments, the RNA construct is a negative strand comprising, from 3′ to 5′, an RdRP-responsive 5′ UTR, a nucleic acid sequence encoding an RdRP (e.g., a Dengue virus NS5 protein or a functional variant or fragment thereof), and an exogenous nucleic acid sequence. In embodiments, the RNA construct has the same orientation as any of the above constructs except that the positions of the 5′ UTR and the 3′ UTR are switched. In some negative control embodiments, the RNA construct is a negative strand comprising, from 3′ to 5′, a nucleic acid sequence encoding an RdRP, an exogenous nucleic acid sequence, and an RdRP-responsive 3′ UTR. In some negative control embodiments, the RNA construct is a negative strand comprising, from 3′ to 5′, a nucleic acid sequence encoding an exogenous nucleic acid sequence, a nucleic acid encoding an RdRP, and an RdRP-responsive 3′ UTR.

In some embodiments, the RNA construct comprises, from 5′ to 3′, a positive strand RdRP-responsive 5′ UTR, a positive strand nucleic acid sequence encoding an RdRP (e.g., a Dengue virus NS5 protein or a functional variant or fragment thereof), and a negative strand exogenous nucleic acid sequence. In some embodiments, this construct produces a higher number of protein copies of the exogenous protein encoded by the exogenous nucleic acid sequence than the number of protein copies of RdRP. In certain embodiments, the RNA construct further comprises a positive strand RdRP-responsive 3′ UTR (e.g., disposed 3′ relative to the negative strand exogenous nucleic acid sequence). In some embodiments, the RNA construct comprises, from 3′ to 5′, a negative strand RdRP-responsive 5′ UTR, a negative strand nucleic acid sequence encoding an RdRP (e.g., a Dengue virus NS5 protein or a functional variant or fragment thereof), and a positive strand exogenous nucleic acid sequence. In certain embodiments, the RNA construct further comprises a negative strand RdRP-responsive 3′ UTR (e.g., disposed 5′ relative to the negative strand exogenous nucleic acid sequence).

In any of the above embodiments, e.g., embodiments in which a UTR does not provide an IRES sequence, an IRES sequence may be included, e.g., situated between the UTR and the coding region (e.g., a coding region for an exogenous protein or an RdRP). In embodiments, the IRES sequence is situated upstream (e.g., immediately upstream) of the coding region.

In any of the above embodiments, a protease cleavage sequence (e.g., a 2A sequence) may be included. e.g., situated between the coding region for the exogenous protein and the coding region for the RdRP. In some embodiments, the nucleic acid construct encodes the gene encoding the exogenous protein and the RdRP, whereby a “self-cleaving” 2A element is disposed between the cistrons encoding the genes. The 2A element is believed to function by making the ribosome skip the synthesis of a peptide bond at the C-terminus of a 2A element, leading to separation between the end of the 2A sequence and the next polypeptide downstream (see, e.g., Holst et al. (2008) Nat. Immunol. 6:658-66). In some embodiments, the gene encoding the exogenous protein is separated from the gene encoding the RdRP by a viral-derived 2A element or a sequence having at least 75% (e.g., at least 75%, 80%, 85%, 90%, 95%, 96%, 97%, 98%, 99%, or 100%) sequence identity thereto. In some embodiments, the construct encodes a 2A protein sequence encoded by the viral-derived 2A element, or a polypeptide sequence with at least 75%, 80%, 85%, 90%, 95%, 96%, 97%, 98%, 99%, or 100% sequence identity thereto. Multiple 2A elements are known in the art and can be used as described herein, including, e.g., T2A. P2A, E2A, and F2A (see, e.g., Liu et al. (2017) Sci. Rep. 7(1): 2193. Additional exemplary 2A element amino acid sequences are provided in Table 9 below.

TABLE 9
Exemplary 2A peptide sequences.
(GSC residues can be optionally added to the
5′ end of each peptide, as shown, e.g.,
to improve cleavage efficiency.)

| Peptide | Amino Add Sequence | SEQ ED NO: |
|---|---|---|
| T2A | (GSG)EGRGSLLTCGDVEENPGP | 40 |
| P2A | (GSG)ATNFSLLKQAGDVEENPGP | 41 |
| E2A | (GSG)QCTNYALLKLAGDVESNPGP | 42 |
| F2A | (GSG)VKQTLNFDLLKLAGDVESNPGP | 43 |

Providing RdRP in Trans

In any of the above embodiments, replication of the RNA construct may involve providing a cell with an RdRP that is compatible with the RdRP-responsive 5′UTR and/or RdRP-responsive 3′UTR of a nucleic acid construct that is also provided to the cell (e.g., an RdRP encoded by a flavivirus NS5 gene). e.g., in trans. In some embodiments, the RdRP is a flavivirus RdRP (e.g., a Dengue virus RdRP), e.g., as described herein. In some embodiments, the RdRP is a non-flavivirus RdRP (e.g., a tomato RdRP or a tobacco RdRP). The RdRP may be provided to the cell as a protein or as a nucleic acid (e.g., DNA (e.g., plasmid DNA) or RNA (e.g., mRNA or an RNA construct)) encoding the RdRP. In some embodiments, a DNA molecule (e.g., a plasmid, cosmid, episome, or artificial chromosome) comprising a sequence encoding the RdRP is introduced into the cell. In some embodiments, an RNA molecule comprising a sequence encoding the RdRP is introduced into the cell. Alternatively or in combination, the cell may be genetically-modified to comprise one or more copies of an RdRP-encoding sequence in its genome, such that it is capable of expressing the RdRP. In some embodiments, an RdRP-encoding gene is stably transfected into the cell. In some embodiments, an RdRP-encoding gene is stably integrating into the genome of the cell. In some embodiments, RdRP protein is introduced into the cell.

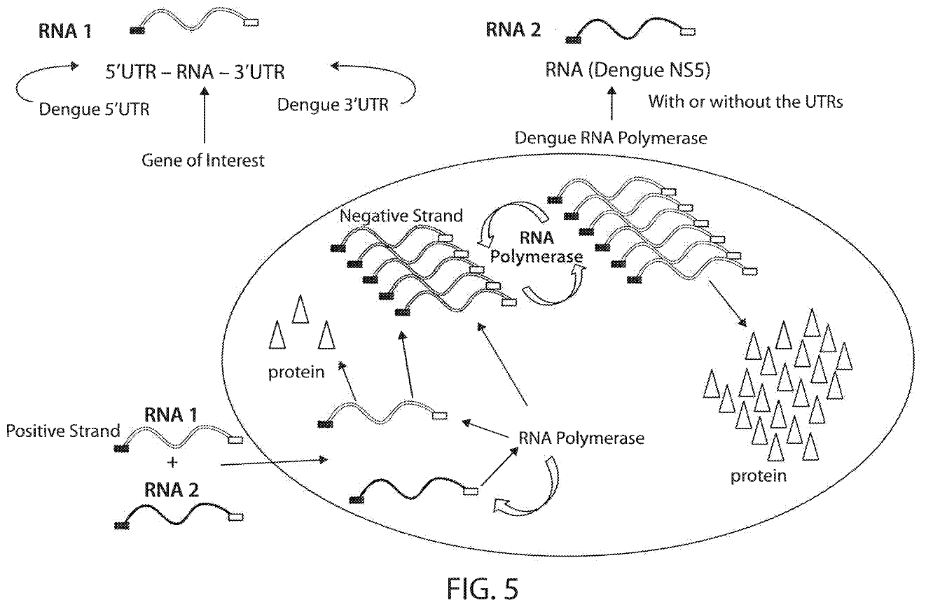

In one set of examples, FIGS. 5 and 6 show exemplary schematics in which an RNA construct encoding a gene of interest flanked by RdRP-responsive UTR sequences (RNA 1) is delivered to a cell alongside an RNA construct encoding a compatible RdRP (RNA 2). The RdRP can thus be translated from RNA 2, and the resultant RdRP protein can then induce amplification of RNA 1 or a complement thereof. In some embodiments, the cell comprises a nucleic acid encoding the compatible RdRP, e.g., in its genomic DNA. Such a cell may be produced, for example, by stably transfecting the cell with a construct comprising an open reading frame encoding the compatible RdRP. e.g., such that the open reading frame encoding the compatible RdRP is stably integrated into the genome of the cell. In some embodiments, compatible RdRP protein can be introduced into a cell using methods known in the art. The RdRP protein can be introduced into the cell, for example, prior to, concurrently with, or after the introduction of the nucleic acid construct to the cell.

In some embodiments, the RdRP is generated in the same cell that comprises the RNA construct, e.g., from a second nucleic acid (e.g., RNA) construct. In some embodiments, providing an RdRP in trans using a second nucleic acid construct is used. e.g., when the RNA construct does not comprise a sequence encoding a functional RdRP. Non-limiting examples of second nucleic acid constructs that can be used to provide an RdRP are shown as FIGS. 1F-1M. In some embodiments, the second nucleic acid construct further comprises an RdRP-responsive 5′ UTR. In some embodiments, the second nucleic acid construct further comprises an RdRP-responsive 3′ UTR. In some embodiments, the second nucleic acid construct further comprises an RdRP-responsive 5′ UTR and an RdRP-responsive 3′ UTR. In some embodiments, the second nucleic acid construct does not comprise an RdRP-responsive 5′ UTR or an RdRP-responsive 3′ UTR. In some embodiments, the second nucleic acid construct is a DNA construct or an RNA construct. In embodiments, the second nucleic acid construct (e.g., a positive strand RNA or a negative strand RNA) comprises a nucleic acid sequence encoding an RdRP (e.g., a Dengue virus NS5 protein or a functional variant or fragment thereof). In embodiments, the second nucleic acid construct is a positive strand RNA comprising, in order from 5′ to 3′, an RdRP-responsive 5′ UTR, a nucleic acid sequence encoding an RdRP (e.g., a Dengue virus NS5 protein or a functional variant or fragment thereof), and an RdRP-responsive 3′ UTR. In embodiments, the second nucleic acid construct is a positive strand RNA comprising, in order from 5′ to 3′, an RdRP-responsive 5′ UTR and a nucleic acid sequence encoding an RdRP (e.g., a Dengue virus NS5 protein or a functional variant or fragment thereof). In embodiments, the second nucleic acid construct is a positive strand RNA comprising, in order from 5′ to 3′, a nucleic acid sequence encoding an RdRP (e.g., a Dengue virus NS5 protein or a functional variant or fragment thereof) and an RdRP-responsive 3′ UTR (e.g., a construct that can be transcribed to produce mRNA encoding the NS5 protein or a functional variant or fragment thereof, but which would not be amplified by the encoded RdRP). In embodiments, the second nucleic acid construct is a negative strand RNA comprising, in order from 3′ to 5′, an RdRP-responsive 5′ UTR, a nucleic acid sequence encoding an RdRP (e.g., a Dengue virus NS5 protein or a functional variant or fragment thereof), and an RdRP-responsive 3′ UTR. In embodiments, the second nucleic acid construct is a negative strand RNA comprising, in order from 3′ to 5′, an RdRP-responsive 5′ UTR and a nucleic acid sequence encoding an RdRP (e.g., a Dengue virus NS5 protein or a functional variant or fragment thereof). In embodiments, the second nucleic acid construct is a negative strand RNA comprising, in order from 3′ to 5′, a nucleic acid sequence encoding an RdRP (e.g., a Dengue virus NS5 protein or a functional variant or fragment thereof) and an RdRP-responsive 3′ UTR (e.g., a construct that can be transcribed to produce mRNA encoding the NS5 protein or a functional variant or fragment thereof, but which would not be amplified by the encoded RdRP).

In any of the above embodiments, e.g., embodiments in which a UTR does not provide an IRES sequence, an IRES sequence may be included, e.g., situated between the UTR and the coding region for the RdRP. In embodiments, the IRES sequence is situated upstream (e.g., immediately upstream) of the coding region.

Providing Helicase in Trans

In any of the embodiments herein, e.g., the embodiments above, replication of the RNA construct may involve providing a cell with a helicase (e.g., a helicase encoded by a flavivirus NS3 gene, e.g., as described herein), e.g., in trans. While not wishing to be bound by theory, in some embodiments, the relatively high conservation of helicases allows a helicase to be used that is not derived from the same organism as the RdRP. The helicase may be provided to the cell as a protein or as a nucleic acid (e.g., DNA (e.g., plasmid DNA) or RNA (e.g., mRNA or an RNA construct)) encoding the helicase. Alternatively or in combination, the cell may be genetically-modified to comprise one or more copies of the helicase in its genome, such that it is capable of expressing the helicase. In other embodiments, a helicase protein can be introduced into a cell using methods known in the art. The helicase can be introduced into the cell, for example, prior to, concurrently with, or after the introduction of the nucleic acid construct to the cell. In some embodiments, the helicase is a flavivirus NS3 protein provided herein.

Additional RNA Constructs, e.g., for Quenching Nucleic Acids or Negative Controls

In some embodiments, the RNA construct is a negative strand comprising, from 5′ to 3′, an RdRP-responsive 3′ UTR and an exogenous nucleic acid sequence. In some embodiments, the RNA construct is a negative strand comprising, from 5′ to 3′, an exogenous nucleic acid sequence and an RdRP-responsive 3′ UTR. In some embodiments, the RNA construct is a positive strand comprising, from 5′ to 3′, an RdRP-responsive 3′ UTR and an exogenous nucleic acid sequence. In some embodiments, the RNA construct is a positive strand comprising, from 5′ to 3′, an exogenous nucleic acid sequence and an RdRP-responsive 3′ UTR. It is contemplated that such constructs would be negative control constructs that would not be replicated in the presence of an RdRP due to the absence of 5′ UTRs.

Exemplary Dengue Virus-Derived Construct Sequences

Exemplary sequences for RNA constructs comprising a Dengue virus UTR sequence and an exogenous nucleic acid sequence encoding eGFP are provided in Table 12. In some embodiments, an RNA construct comprises one or more exemplary Dengue virus sequences, e.g., as listed in Table 1. For example, one exemplary construct consists of a positive strand comprising a Dengue virus 5′ UTR, a nucleic acid sequence encoding eGFP, and a Dengue virus 3′ UTR (SEQ ID NO: 1). Another exemplary construct consists of a negative strand comprising a Dengue virus 5′ UTR, a nucleic acid sequence encoding eGFP, and a Dengue virus 3′ UTR (SEQ ID NO: 2). Another exemplary construct consists of a negative strand comprising a Dengue virus 5′ UTR and a nucleic acid sequence encoding eGFP (SEQ ID NO: 3). Another exemplary construct consists of a negative strand comprising a nucleic acid sequence encoding eGFP and a Dengue virus 3′ UTR (SEQ ID NO: 4).

Other Flavivirus-Based Constructs

It is contemplated that any of the above arrangements described, e.g., for a Dengue virus RNA construct, can be applied for any flavivirus-based RNA construct. e.g., by replacing the Dengue virus elements with the corresponding elements from another flavivirus.

### Other Viruses

It is contemplated that elements of the nucleic acid constructs (e.g., RdRPs, helicases (e.g., flavivirus helicases, e.g., as described herein), RdRP-responsive 5′ UTRs and RdRP responsive 3′ UTRs) of the nucleic acid constructs described herein can be replaced with corresponding elements from another virus that encodes an RdRP. In some embodiments, the element to be replaced comprises an RdRP-responsive 5′ UTR, an RdRP-responsive 3′ UTR, a helicase-encoding gene (e.g., a gene encoding a flavivirus helicase, e.g., an NS3 protein as described herein, or a non-flavivirus helicase), or an RdRP-encoding gene (e.g., a gene encoding a flavivirus RdRP, e.g., NS5). In some embodiments, a nucleic acid construct comprising a RdRP-responsive 5′ UTR from a particular viral clade may be amplified in the presence of a compatible RdRP, e.g., an RdRP from the same viral clade. In some embodiments, a nucleic acid construct comprising a RdRP-responsive 3′ UTR from a particular viral clade may be amplified in the presence of a compatible RdRP, e.g., an RdRP from the same viral clade. Examples of viral clades known to include RdRPs (corresponding RdRP-responsive 5′ UTRs and RdRP responsive 3′ UTRs, and optionally a helicase) are known in the art and include, without limitation. Flaviviridae, Birnaviridae, Cystoviridae, Reoviridae, Hypoviridae, Partitiviridae, Totiviridae, Mononegavirales. Leviviridae, Cystoviridae, Reoviridae, Orthomyxoviridae (e.g., influenza A, B, and C viruses, Thogotoviruses, and the infectious salmon anemia virus), Arenaviridae. Bunyaviridae (e.g., Phenuiviridae), Hantaviridae, Nairoviridae, Phleboviridae, Tenuivirus, and Tospoviridae. Exemplary viruses that encode RdRPs include, without limitation, Dengue virus (encoding RdRP NS5). Kunjin virus, West Nile virus, tick-borne encephalitis virus, yellow fever virus, Zika virus, cell fusing agent virus (CFAV), Palm Creek virus (PCV), and Parramatta River virus (PaRV), poliovirus (encoding RdRP 3Dpol), vesicular stomatitis virus (encoding RdRP L), hepatitis C virus (encoding RdRP NS5B), mengovirus, influenza A. B, and C viruses. Thogotoviruses, and the infectious salmon anemia virus. In some embodiments, the virus is not an alphavirus.

### Exemplary Genes of Interest

One or more exogenous genes of interest may be included in a nucleic acid construct described herein (e.g., in an exogenous nucleic acid). In some embodiments, the nucleic acid construct comprises one gene of interest. In some embodiments, the nucleic acid construct comprises a plurality of genes of interest (e.g., 2, 3, 4, 5, 6, 7, 8, 9, or 10 genes of interest). In some embodiments, each of the plurality of genes of interest is a distinct gene (e.g., encoding a distinct protein or functional RNA molecule). Generally, the exogenous gene of interest is to be expressed in a cell to which the nucleic acid construct is introduced. The gene of interest may be exogenous to the cell and/or exogenous to the virus from which the 5′ UTR of the nucleic acid construct was derived. In some embodiments, the exogenous gene of interest encodes an exogenous polypeptide (e.g., an exogenous polypeptide comprising a therapeutic agent). In some embodiments, the exogenous gene of interest encodes an exogenous protein (e.g., an antibody molecule, enzyme, cytokine, cytokine receptor, chemokine, chemokine receptor, interleukin, receptor, ligand, hormone, growth factor, blood factor, lysosomal storage enzyme, immune stimulatory molecule (e.g., a costimulatory molecule), intein, DNA-binding protein. RNA-binding protein, a complement regulatory molecule, a complement cascade molecule, a clotting cascade molecule, a chelator, or a functional fragment thereof). In certain embodiments, the enzyme comprises asparaginase, a protease, a nuclease, a glycosidase, a lipase, or a DNase. In certain embodiments, the antibody molecule comprises a whole antibody or a fragment thereof, single-chain antibody, humanized antibody; murine antibody; chimeric, mouse-human, mouse-primate, primate-human monoclonal antibody, anti-idiotype antibody, or an antibody fragment, such as, e.g., scFv. (scFv)2. Fab, Fab′, and F(ab′)2, F(ab1)2, Fv, dAb, and Fd fragments, diabodies, duobodies, and an antibody-related polypeptide. In certain embodiments, the antibody molecule comprises a multispecific (e.g., bispecific) antibody. In some embodiments, the exogenous gene of interest encodes an exogenous protein listed in PCT Publication No. WO 2018/151829, incorporated herein by reference. Additional non-limiting examples of exogenous proteins are found, e.g., in WO2015/073587, WO2015/153102, and WO2016/183482, each of which is incorporated by reference in its entirety.

In some embodiments the exogenous gene of interest encodes an exogenous nucleic acid, e.g., a DNA or RNA (e.g., a functional RNA, e.g., a microRNA, ribozyme, aptamer, siRNA, piRNA, snoRNA, snRNA, long ncRNA, or tRNA).

In some embodiments, the exogenous gene of interest is a mammalian gene, e.g., primate gene, e.g., human gene. In some embodiments, the exogenous gene of interest comprises a fungal gene, bacterial gene (e.g., a gene encoding an asparaginase), plant gene, viral gene, or prion gene. In embodiments, the exogenous gene of interest is a wild-type gene. In some embodiments, the exogenous gene of interest encodes a wild-type protein. The gene may be codon-optimized. The gene may encode a therapeutic agent of interest, such that a cell (e.g., an erythroid cell) may be modified to express the therapeutic agent of interest according to the methods herein. The cell may then be used as a vehicle for delivering the therapeutic agent of interest into a subject in need thereof (e.g., according to the methods described herein).

### Exemplary Polypeptides

An erythroid cell described herein may comprise an exogenous protein, e.g., expressed from an amplifiable RNA as described herein. An exemplary exogenous protein, e.g., may include:

a) a naturally occurring form of the protein;

b) the protein having a sequence appearing in a database, e.g., GenBank database, on Jan. 10, 2018;

c) a protein having a sequence that differs by no more than 1, 2, 3, 4, 5 or 10 amino acid residues from a sequence of a) or b);

d) a protein having a sequence that differs at no more than 1, 2, 3, 4, 5 or 10% its amino acids residues from a sequence of a) or b);

e) a protein having a sequence that does not differ substantially from a sequence of a) or b); or

f) a protein having a sequence of c), d), or e) that does not differ substantially in a biological activity, e.g., an enzymatic activity (e.g., specificity or turnover) or binding activity (e.g., binding specificity or affinity) from a protein having the sequence of a) or b).

In embodiments, the protein comprises a protein or fragment thereof, e.g., all or a fragment of a protein of a), b), c), d), e), or f) of the preceding paragraph. In embodiments, the protein comprises an exogenous protein described herein, or an active protein having an amino acid sequence identity of at least 70%, 75%, 80%, 85%, 90%, 95%, 96%, 97%, 98%, or 99% identity thereto.

In some embodiments, an exogenous polypeptide described herein is at least 200, 300, 400, 500, 600, 700, or 800 amino acids in length. In some embodiments, the exogenous polypeptide is between 200-300, 300-400, 400-500, 500-600, 600-700, or 700-800 amino acids in length.

In some embodiments, an erythroid cell, e.g., an enucleated erythroid cell, comprises at least 1,000, 5,000, 10,000, 15,000, 20,000, 25,000, 30,000, 50,000, 100,000, 200,000, or 500,000 copies of an exogenous polypeptide described herein.

In some embodiments, the exogenous protein described herein comprises a leader sequence (e.g., a naturally-occurring leader sequence or a synthetic leader sequence). In some embodiments, the exogenous protein lacks a leader sequence (e.g., is genetically modified to remove a naturally-occurring leader sequence). In some embodiments, the exogenous protein comprises an N-terminal methionine residue. In some embodiments, the exogenous protein lacks an N-terminal methionine residue. In some embodiments, a naturally-occurring protein (e.g., a viral protein) lacking an N-terminal methionine residue can be designed to include an N-terminal methionine residue for expression in a cell.

In some embodiments, the exogenous protein comprises an antibody molecule, enzyme, cytokine, cytokine receptor, chemokine, chemokine receptor, interleukin, receptor, ligand, hormone, growth factor, blood factor, lysosomal storage enzyme, immune stimulatory molecule (e.g., a costimulatory molecule), intein. DNA-binding protein, RNA-binding protein, a complement regulatory molecule, a complement cascade molecule, a clotting cascade molecule, a chelator, or a functional fragment thereof. In certain embodiments, the enzyme comprises asparaginase, a protease, a nuclease, a glycosidase, a lipase, or a DNase.

In certain embodiments, the exogenous protein comprises an enzymatic recognition sequence, a transpeptidase recognition sequence, a protease recognition sequence, a cleavable domain, a complement regulatory domain, an SCR domain, a CCP domain, an immunoglobulin or immunoglobulin-like domain, an armadillo repeat, a leucine zipper, a death effector domain, a cadherin repeat, an EF hand, a phosphotyrosine binding domain, a pleckstrin homology domain, an SCR homology 2 domain, a zinc finger domain, a cyclic peptide, a cell-penetrating peptide, a chaperone molecule, an integrin, a collagen, a carrier protein (e.g., albumin), a toxin binding peptide (e.g., a peptide that binds to a toxin from a bacterium, a parasite, a fungus or the environment), a myelination molecule, a prion protein binding molecule, a cluster of differentiation (CD) molecule, an immunomodulatory molecule (e.g., a co-stimulatory molecule, an activator of a co-stimulatory molecule, an inhibitor of a co-stimulatory molecule, a co-inhibitory molecule, an inhibitor of a co-inhibitory molecule or an activator of a co-inhibitory molecule), a cancer antigen or cancer cell marker, an antigen-presenting molecule, a pro-apoptotic molecule, a targeting moiety, an Fc receptor binding molecule, a tumor starvation enzyme, a DNA damage inhibitor, a cell-cycle inhibitor, a flexible linker, or an epitope tag. In certain embodiments, the exogenous protein comprises an extracellular domain, counterligand binding domain, or other biologically active domain, or a fragment or variant thereof. In some embodiments, the exogenous protein comprises an antigen, e.g., a tumor antigen, infectious disease antigen, or autoantigen.

In some embodiments, the exogenous polypeptide is a protein comprising a transmembrane domain (e.g., a Type I, Type II, or Type III transmembrane domain). In embodiments, the transmembrane domain comprises a transmembrane domain present endogenously in mammalian. e.g., human, erythroid cells. e.g., erythrocytes. In embodiments, the exogenous polypeptide comprises a signal sequence that directs the protein to the cell surface. e.g., to display a signaling domain and/or a binding domain on the exterior of the cell.

In some embodiments, the exogenous polypeptide comprises a secretory signal peptide. In some embodiments, the exogenous polypeptide is secreted by the cell.

### Antibody Molecules

The present disclosure provides nucleic acid constructs comprising a gene of interest. In some embodiments, the gene of interest encodes an antibody molecule.

In some embodiments, the antibody molecule comprises one or more CDRs, e.g., one or more of a heavy chain CDR1, a heavy chain CDR2, a heavy chain CDR3, a light chain CDR1, a light chain CDR2, or a light chain CDR3. In some embodiments, the antibody molecule comprises a heavy chain CDR3 (e.g., in the absence of other CDRs). In some embodiments, the antibody molecule comprises a heavy chain CDR1, a heavy chain CDR2, and a heavy chain CDR3. In some embodiments, light chain CDRs are not present. In some embodiments, the antibody molecule comprises one or more of (e.g., 2 or 3 of) a light chain CDR1, a light chain CDR2, and a light chain CDR3 (e.g., in addition to the three heavy chain CDRs). In some embodiments, the antibody molecule comprises a heavy chain CDR1, a heavy chain CDR2, a heavy chain CDR3, a light chain CDR1, a light chain CDR2, and a light chain CDR3.

The antibody molecule may comprise an immunoglobulin. Depending on the antibody amino acid sequence of the constant domain of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4. IgA1 and IgA2. The heavy-chain constant domains that correspond to the different classes of immunoglobulins are called alpha, delta, epsilon, gamma, and mu, respectively. The subunit structures and three-dimensional configurations of different classes of immunoglobulins are well known. In some embodiments, the antibody molecule is or comprises an antibody fragment (e.g., antigen-binding fragment) such as an Fv fragment, a Fab fragment, a F(ab′)2 fragment, and a Fab′ fragment. Additional examples of antibody fragments include an antigen-binding fragment of an IgG (e.g., an antigen-binding fragment of IgG1, IgG2, IgG3, or IgG4) (e.g., an antigen-binding fragment of a human or humanized IgG, e.g., human or humanized IgG1, IgG2, IgG3, or IgG4); an antigen-binding fragment of an IgA (e.g., an antigen-binding fragment of IgA1 or IgA2) (e.g., an antigen-binding fragment of a human or humanized IgA, e.g., a human or humanized IgA1 or IgA2): an antigen-binding fragment of an IgD (e.g., an antigen-binding fragment of a human or humanized IgD); an antigen-binding fragment of an IgE (e.g., an antigen-binding fragment of a human or humanized IgE); or an antigen-binding fragment of an IgM (e.g., an antigen-binding fragment of a human or humanized IgM). The antibody molecule may be of any class, such as IgG. IgA, or IgM (or sub-class thereof), and the antibody molecule need not be of any particular class.

Examples of antibody molecules include, but are not limited to, Fab, Fab′, F(ab′)₂, Fv fragments, scFv antibody fragments, disulfide-linked Fvs (sdFv), a Fd fragment consisting of the VH and CH1 domains, linear antibodies, single domain antibodies such as sdAb (either VL or VH), camelid VHH domains, multi-specific antibodies formed from antibody fragments such as a bivalent fragment comprising two Fab fragments linked by a disulfide bridge at the hinge region, an isolated epitope binding fragment of an antibody, maxibodies, minibodies, nanobodies, intrabodies, diabodies, triabodies, tetrabodies, v-NAR and bis-scFv.

In some embodiments, the antibody molecule is a multispecific antibody molecule, e.g., a bispecific antibody molecule.

In some embodiments, the antibody molecule is a humanized antibody, a chimeric antibody, a multivalent antibody, or a fragment thereof. In some embodiments, the antibody molecule is a scFv-Fc (see, e.g., Sokolowska-Wedzina et al., Mol. Cancer Res. 15(8):1040-1050, 2017), a VHH domain (see, e.g., Li et al., Immunol. Lett. 188:89-95, 2017), a VNAR domain (see. e.g., Hasler et al., Mol. Immunol. 75:28-37, 2016), a (scFv)₂, a minibody (see. e.g., Kim et al., PLoS One 10(1):el 13442, 2014), or a BiTE. In some embodiments, the antibody molecule is a DVD-Ig (see, e.g., Wu et al., Nat. Biotechnol. 25(11):1290-1297, 2007; WO 08/024188; and WO 07/024715), or a dual-affinity re-targeting antibody (DART) (Tsai et al., Mol. Ther. Oncolytics 3:15024, 2016), a triomab (see, e.g., Chelius et al., MAbs 2(3):309-319, 2010), kih IgG with a common LC (see. e.g., Kontermann et al., Drug Discovery Today 20(7):838-847, 2015), a crossmab (see, e.g., Regula et al., EMBO Mol. Med. 9(7):985, 2017), an ortho-Fab IgG, a 2-in-1-IgG, IgG-scFv (see. e.g., Cheat et al., Mol. Cancer Ther. 13(7):1803-1812, 2014), scFv2-Fc (see, e.g., Natsume et al., J. Biochem. 140(3):359-368, 2006), a bi-nanobody, tandem antibody, a DART-Fc, a scFv-HSA-scFv, a DNL-Fab3, a DAF (two-in-one or four-in-one), a DutaMab, a DT-IgG, a knobs-in-holes common LC, a knobs-in-holes assembly, a charge pair antibody, a Fab-arm exchange antibody, a SEEDbody, a Triomab, a LUZ-Y, a Fcab, a kλ-body, a orthogonal Fab, a DVD-IgG, a IgG(H)-scFv, a scFv-(H)IgG, a IgG(L)-scFv, a scFv-(L)-IgG, a IgG (L,H)-Fc, a IgG(H)-V, a V(H)-IgG, a IgG(L)-V, a V(L)-IgG, a KIH IgG-scFab, a 2scFv-IgG, a IgG-2scFv, a scFv4-Ig, a Zybody, a DVI-IgG, a nanobody (e.g., antibodies derived from Camelus bactriamus, Calelus dromaderius, or Lama paccos) (see, e.g., U.S. Pat. No. 5,759,808; and Stijlemans et al., J. Biol. Chem. 279:1256-1261, 2004; Dumoulin et al., Nature 424:783-788, 2003; and Pleschberger et al., Bioconjugate Chem. 14:440-448, 2003), a nanobody-HSA, a diabody (see, e.g., Poljak. Structure 2(12):1121-1123, 1994; and Hudson et al., J. Immunol. Methods 23(1-2):177-189, 1999), a TandAb (see, e.g., Reusch et al., mAbs 6(3):727-738, 2014), a scDiabody (see, e.g., Cuesta et al., Trends in Biotechnol. 28(7):355-362, 2010), a scDiabody-CH3 (see, e.g., Sanz et al., Trends in Immunol. 25(2):85-91, 2004), a diabody-CH3, a Triple Body, a miniantibody, a minibody, a TriBi minibody, a scFv-CH3 KIH, a Fab-scFv, a scFv-CH-CL-scFv, a F(ab′)2-scFV2, a scFv-KIH, a Fab-scFv-Fe, a tetravalent HCAb, a scDiabody-Fc, a diabody-Fc, a tandem scFv-Fc, an intrabody (see, e.g., Huston et al., Human Antibodies 10(3-4):127-142, 2001; Wheeler et al., Mol. Ther. 8(3):355-366, 2003; and Stocks, Drug Discov. Today 9(22):960-966, 2004), a dock and lock bispecific antibody, an ImmTAC, a HSAbody, a scDiabody-HSA, a tandem scFv, an IgG-IgG, a Cov-X-Body, and a scFv1-PEG-scFv2. In some embodiments, the antibody molecule can be an IgNAR, a bispecific antibody (see. e.g., Milstein and Cuello, Nature 305:537-539, 1983; Suresh et al., Methods in Enzymology 121:210, 1986; WO 96/27011; Brennan et al., Science 229:81, 1985; Shalaby et al., J. Exp. Med. 175:217-225, 1992; Kolstelny et al., J. Immunol. 148(5):1547-1553, 1992; Hollinger et al., Proc. Natl. Acad. Sci. U.S.A. 90:6444-6448, 1993; Gruber et al., J. Immunol. 152:5368, 1994: and Tuft et al., J. Immunol. 147:60, 1991), a bispecific diabody, a triabody (Schoonooghe et al., BMC Biotechnol. 9:70, 2009), a tetrabody, a scFv-Fc knobs-into-holes, a scFv-Fc-scFv, a (Fab′scFv)2, a V-IgG, a IvG-V, a dual V domain IgG, a heavy chain immunoglobulin or a camelid (Holt et al., Trends Biotechnol. 21(11):484-490, 2003), an intrabody, a heteroconjugate antibody (e.g., U.S. Pat. No. 4,676,980), a linear antibody (Zapata et al., Protein Eng. 8(10:1057-1062, 1995), a trispecific antibody (Tuft et al., J. Immunol. 147:60, 1991), a Fabs-in-Tandem immunoglobulin (WO 15/103072), or a humanized camelid antibody. In some embodiments, the antibody molecule is a synthetic antibody (also known as an antibody mimetic) (see. e.g., Yu et al. (2017) Annu. Rev. Anal. Chem. (Palo Alto Calif.) 10(1): 293-320; and Hey et al. (2005) Trends Biotechnol. 23(10): 514-22). For example, in some embodiments, the antibody molecule comprises an adnectin, an affibody, an affilin, an affimer, an affitin, an alphabody, an anticalin, an aptamer, an armadillo repeat protein-based scaffold, an atrimer, an avimer, a DARPin, a fynomer, a knottin, a Kunitz domain peptide, a monobody or a nanofitin.

### Methods of Making Cells Expressing Exogenous Genes

Cells expressing exogenous genes of interest (e.g., therapeutic agents) can be produced using the compositions described herein. Generally, a nucleic acid construct described herein (e.g., an RNA construct described herein) comprising an exogenous nucleic acid sequence is introduced into a cell (e.g., an erythroid cell). In certain embodiments, the nucleic acid construct is an RNA construct as described herein, which can be introduced into the cell. e.g., by electroporation or in a lipid nanoparticle. In certain embodiments, the nucleic acid construct is a DNA (e.g., a vector, e.g., a viral vector, e.g., a lentiviral vector) comprising a nucleic acid sequence encoding an RNA construct as described herein, which can be introduced into the cell, e.g., by electroporation, transfection, or in a lipid nanoparticle (e.g., lipid nanoparticle-mediated transfection).

If an RdRP is present in the cell, the nucleic acid construct may be amplified in the cell. In some embodiments, the nucleic acid construct comprises an RdRP-responsive 5′ UTR (e.g., a flavivirus 5′ UTR) and an RdRP-responsive 3′ UTR (e.g., a flavivirus 3′ UTR) and is exponentially amplified in the cell by the RdRP. In some embodiments, the nucleic acid construct comprises an RdRP-responsive 5′ UTR (e.g., flavivirus 5′ UTR), but does not comprise an RdRP-responsive 3′ UTR (e.g., flavivirus 3′ UTR), and is linearly amplified in the cell by the RdRP. The RdRP can be expressed from the nucleic acid construct or can be supplied in trans, e.g., as described herein.

In some embodiments, the cell is then cultured under conditions whereby the exogenous nucleic acid sequence is expressed, thereby producing an exogenous product (e.g., an exogenous protein or an exogenous functional RNA). In embodiments, the exogenous product is a therapeutic agent. In embodiments, the culturing results in enucleation of the cell. The cultured cell can, in some embodiments, be prepared for administration to a subject. e.g., to treat a disease or disorder treatable with the exogenous product.

In some embodiments, an erythroid cell precursor is transfected with an RNA construct comprising an exogenous nucleic acid sequence as described herein. The erythroid cell precursor can then be incubated under conditions that allow differentiation of the cell, e.g., into an enucleated erythroid cell. e.g., as described herein. The enucleated erythroid cell can be prepared for administration to a subject. e.g., to treat a disease or disorder treatable with a product (e.g., a protein or functional RNA) encoded by the exogenous nucleic acid and expressed by the erythroid cell.

The nucleic acid construct (e.g., an RNA) can be introduced into the cell (e.g., a nucleated cell precursor) using at various timepoints that support expression.

In some embodiments, a method of making an erythroid cell comprising an RNA described herein (e.g., an RNA comprising an RdRP-dependent 5′ UTR and/or 3′ UTR), comprises:

a) providing an erythroid cell in maturation phase. e.g., as defined in International Application WO2018/009838, which is herein incorporated by reference in its entirety, and

b) contacting the erythroid cell with an mRNA encoding the exogenous protein, under conditions that allow uptake of the mRNA by the erythroid cell,

thereby making an erythroid cell comprising an mRNA encoding an exogenous protein.

In embodiments, the method comprises providing a population of erythroid cells in maturation phase and contacting a plurality of cells of the population of erythroid cells with the RNA described herein. In embodiments, the population of erythroid cells in maturation phase is a population of cells expanded in a maturation medium for 3-7 days, e.g., 4-5 or 4-6 days, e.g., a maturation medium described in International Application WO2018/009838. In embodiments, the population of erythroid cells is a population of erythroid cells comprising one or more (e.g., 2, 3, 4, 5, 6, 7, 8, 9, 10, 11, 12, 13, 14, 15, 16, 17, or more) of the following properties:

i.a) 2-40%, 3-33%, 5-30%, 10-25%, or 15-20% of the cells in the population are enucleated;

i.b) greater than 0%, 0.1%, 0.2%, or 0.5%, but less than 2%, 3%, 4%, or 5% of the cells in the population are enucleated;

i.c) greater than 0%, 0.1%, 0.2%, or 0.5%, but less than 6%, 10%, 15%, 20%, or 25% of the cells in the population are enucleated;

i.d) greater than 0%, 0.1%, 0.2%, or 0.5%, but less than 30%, 35%, 40%, 45%, or 50% of the cells in the population are enucleated;

i.e) no more than 1%, 2%, 3%, 5%, 10%, 15%, 20%, or 30% of the cells in the population are enucleated;

i.f) no more than 25%, 30%, 35%, 40%, 45%, or 50% of the cells in the population are enucleated;

i.g) the population of cells has reached 6-70%, 10-60%, 20-50%, or 30-40% of maximal enucleation;

i.h) the population of cells has reached no more than 1%, 2%, 3%, 5%, %, 6%, 10%, 15%, 20%, 30%, 40%, 50%, or 60% of maximal enucleation;

i.i) the population of cells has reached no more than 25%, 30%, 35%, 40%, 45%, 50%, or 60% of maximal enucleation;

ii.a) the population of cells is fewer than 3, 2, or 1 population doubling from a plateau in cell division:

ii.b) the population of cells is capable of fewer than 3, 2, or 1 population doubling;

ii.c) the population will increase by no more than 1.5, 2, or 3 fold before the population reaches an enucleation level of at least 70% of cells in the population;

iii.a) at least 80%, 85%, 90%, 95%, or 99% of the cells in the population are normoblasts (e.g., polychromatic or orthochromatic normoblasts);

iii.b) at least 50%, 60%, 70%, 75%, or 79% of the cells in the population are normoblasts (e.g., polychromatic or orthochromatic normoblasts);

iii.c) 30-90%, 40-90%, 50-90%, 60-90%, or 70-90% of the cells in the population are normoblasts (e.g., polychromatic or orthochromatic normoblasts);

iii.d) at least 80%, 85%, 90%, 95%, or 99% of the cells in the population exhibit the morphology of a normoblast (e.g., a polychromatic or orthochromatic normoblast);

iii.e) at least 50%, 60%, 70%, 75%, or 79% of the cells in the population exhibit the morphology of a normoblast (e.g., a polychromatic or orthochromatic normoblast);

iii.f) 30-90%, 40-90%, 50-90%, 60-90%, or 70-90% of the cells in the population exhibit the morphology of a normoblast (e.g., a polychromatic or orthochromatic normoblast);

iii.g) at least 50%, 60%, 70%, 80%, 90%, or 30-90%, 40-90%, 50-90%, 60-90%, or 70-90% of the cells in the population are, or exhibit the morphology of, a hematopoietic stem cell;

iii.h) at least 50%, 60%, 70%, 80%, 90%, or 30-90%, 40-90%, 50-90%, 60-90%, or 70-90% of the cells in the population are, or exhibit the morphology of, a common myeloid progenitor;

iii.i) at least 50%, 60%, 70%, 80%, 90%, or 30-90%, 40-90%, 50-90%, 60-90%, or 70-90% of the cells in the population are, or exhibit the morphology of, a CFU-S cell;

iii.j) at least 50%, 60%, 70%, 80%, 90%, or 30-90%, 40-90%, 50-90%, 60-90%, or 70-90% of the cells in the population are, or exhibit the morphology of, a BFU-E cell;

iii.k) at least 50%, 60%, 70%, 80%, 90%, or 30-90%, 40-90%, 50-90%, 60-90%, or 70-90% of the cells in the population are, or exhibit the morphology of, a CFU-E cell;

iii.l) at least 50%, 60%, 70%, 80%, 90%, or 30-90%, 40-90%, 50-90%, 60-90%, or 70-90% of the cells in the population are, or exhibit the morphology of, a pronormoblast;

iv.a) the population of cells has a translational activity of at least 600,000, 800,000, 1,000,000, 1,200,000, 1,400,000, 1,600,000, 1,800,000, 2,000,000, 2,200,000, or 2,400,000 as measured by a BONCAT assay, e.g., by a translation assay of Example 10 of International Application WO2018/009838;

iv.b) the population of cells has a translational activity of 600,000-2,400,000, 800,000-2,200,000, 1,000,000-2,000,000, 1,200,000-1,800,000, or 1,400,000-1,600,000 as measured by a BONCAT assay. e.g., by a translation assay of Example 10 of International Application WO2018/009838;

iv.c) the population of cells in maturation phase has at least 10%, 20%, 30%, 40%, 50%, 60%, 70%, 80%, or 90% of maximal translational activity, wherein maximal translational activity refers to the maximal translational activity of a similar number of precursors or progenitors of the cells in maturation phase. e.g., CD34+ cells;

v.a) between 0.1-25% of the cells in the population are enucleated and the population of cells is fewer than 1, 2 or 3 population doublings from a plateau in cell division;

vi.a) 84-99%, 85-95%, or about 90% of the cells in the population are GPA-positive (e.g., as measured by a flow cytometry assay, e.g., a flow cytometry assay of Example 10 of International Application WO2018/009838);

vi.b) at least 84%, 85%, 90%, 95%, or 99% of the cells in the population are GPA-positive (e.g., as measured by a flow cytometry assay, e.g., a flow cytometry assay of Example 10 of International Application WO2018/009838);

vi.c) 54-99%, 55-98%, 60-95%, 65-90%, 70-85%, or 75-80% of the cells in the population are band3-positive (e.g., as measured by a flow cytometry assay, e.g., a flow cytometry assay of Example 10 of International Application WO2018/009838);

vi.d) at least 54%, 55%, 60%, 65%, 70%, 75%, 80%, 85%, 90%, 95%, 98%, or 99% of the cells in the population are band3-positive (e.g., as measured by a flow cytometry assay, e.g., a flow cytometry assay of Example 10 of International Application WO2018/009838);

vi.e) 96-100%, 97-99%, or about 98% of the cells in the population are alpha4 integrin-positive (e.g., as measured by a flow cytometry assay, e.g., a flow cytometry assay of Example 10 of International Application WO2018/009838);

vi.f) at least 95%, 96%, 97%, 98%, or 99% of the cells in the population are alpha4 integrin-positive (e.g., as measured by a flow cytometry assay, e.g., a flow cytometry assay of Example 10 of International Application WO2018/009838);

vi.g) at least 50% (e.g., at least 60%, 70%, 80%, 85%, 90%, 92%, 94%, 96%) of the cells in the population are alpha4 integrin-positive and band3-positive; or

vi.h) at least 50% of the cells in the population are band3-positive and at least 90%-95% are alpha4 integrin-positive.

In some embodiments, the contacting is performed when the population of differentiating erythroid cells is between 0.1 and 25% enucleated (e.g., between 0.1 and 20% enucleated, between 0.1 and 15% enucleated, between 0.1 and 12% enucleated, or between 0.1 and 10% enucleated). In some embodiments, the contacting is performed when the population of differentiating erythroid cells has fewer than 3, 2, or 1 population doubling before a plateau in cell division. In some embodiments, the contacting is performed when at least 50% (at least 60%, 70%, 75%, 80%, 90%, or 95%) of the differentiating erythroid cells exhibit the morphology of a normoblast (e.g., a polychromatic or orthochromatic normoblast). In embodiments, the contacting comprises performing electroporation.

In some embodiments, a cell is genetically modified to express an RdRP and/or a helicase, e.g., by inserting a gene encoding the RdRP and/or helicase into the genome of the cell (e.g., as described herein). A nucleic acid construct of the present disclosure may then be introduced into the cell, thereby causing the cell to express a gene of interest encoded by the nucleic acid construct.

In some embodiments, a nucleic acid molecule encoding an RdRP and/or a helicase is introduced into the cell, thereby driving expression of the RdRP and/or helicase in the cell. In certain embodiments, the nucleic acid molecule is an mRNA encoding the RdRP or helicase. In certain embodiments, the nucleic acid molecule is a DNA comprising a gene encoding the RdRP and/or a gene encoding the helicase. In certain embodiments, the RdRP and/or helicase are constitutively expressed from the DNA. In certain embodiments, the RdRP and/or helicase are under the control of a tissue-specific, cell-specific, and/or inducible promoter on the DNA.

In some embodiments, a plurality of distinct nucleic acid constructs of the present disclosure are introduced into the cell. In certain embodiments, the nucleic acid constructs each comprise the same UTRs (e.g., RdRP-responsive 5′ or 3′ UTRs as described herein). In certain embodiments, the nucleic acid constructs comprise different RdRP-responsive UTRs that respond to different RdRPs (e.g., a plurality of distinct RdRPs present in the cell).

In some embodiments, a negative strand form of an RNA construct (e.g., as described herein) is introduced into a cell. Without wishing to be bound by theory, this may advantageously delay the expression of the gene encoded by the RNA construct until an RdRP present in the cell converts it to the corresponding positive strand.

In some embodiments, a positive strand form of an RNA construct (e.g., as described herein) is introduced into a cell. Without wishing to be bound by theory, this may advantageously increase the speed at which expression of the gene encoded by the RNA construct occurs, as the gene can be translated into protein product immediately upon entry of the positive strand into the cell.

In some embodiments, a nucleic acid construct of the present disclosure is a linear nucleic acid (e.g., a linear RNA) or a circular nucleic acid (e.g., a circular RNA). In certain embodiments, the nucleic acid construct is a circular RNA. Use of such a nucleic acid construct may expedite replication by a compatible RdRP in the cell.

In some embodiments, an RdRP is introduced into the cell prior to, concurrently with, or after the introduction of a nucleic acid construct (e.g., as described herein) into the cell. In some embodiments, a helicase is introduced into the cell prior to, concurrently with, or after the introduction of a nucleic acid construct (e.g., as described herein) into the cell.

### Delivery to a Cell

The components, e.g., an RNA molecule, an RdRP, and/or a helicase described herein, can be delivered to cells in a variety of forms (see, e.g., Table 10 below). For instance, each of the components can be delivered by transfection of the target cell with a nucleic acid molecule (e.g., a DNA plasmid) encoding each of the components (e.g., an RNA construct and/or an RdRP).

TABLE 10
Exemplary Cell Delivery Methods
Components

| RdRP | RNA construct | Comments |
|---|---|---|
| DNA | DNA | In some embodiments, an RdRP and an RNA construct |
| | | described herein (e.g., comprising a RdRP-responsive 5′- |
| | | UTR, at least one gene of interest, and a 3′UTR) are encoded |
| | | (e.g., transcribed and/or translated) by a DNA molecule that is |
| | | delivered to a cell. In some embodiments, an RdRP and an |
| | | RNA molecule described herein are encoded by the same |
| | | DNA molecule. In some embodiments, an RdRP and an |
| | | RNA molecule described herein are encoded by separate |
| | | DNA molecules. |
| DNA | RNA | In some embodiments, an RdRP described herein is encoded |
| | | (e.g., transcribed and translated) by a DNA molecule, and |
| | | both the DNA molecule and an RNA construct (e.g., mRNA) |
| | | described herein are delivered to a cell. |
| RNA | DNA | In some embodiments, an RdRP described herein is encoded |
| | | by an RNA molecule (e.g., an mRNA), and an RNA construct |
| | | described herein (e.g., comprising a RdRP-responsive 5′- |
| | | UTR, at least one gene of interest, and a 3′UTR) is encoded |
| | | (e.g., transcribed and/or translated) by a DNA molecule, and |
| | | both the RNA molecule and the DNA molecule are delivered |
| | | to a cell. In some embodiments the RNA molecule encoding |
| | | the RdRP (e.g., an mRNA) comprises a RdRP-responsive 5′- |
| | | UTR and a 3′UTR. |
| RNA | RNA | In some embodiments, an RdRP described herein is encoded |
| | | by an RNA molecule (e.g,, an mRNA), and both the RNA |
| | | molecule encoding the RdRP and an RNA construct (e.g., an |
| | | mRNA) described herein are delivered to a cell. |
| Protein | DNA | In some embodiments, the RdRP is provided as a protein, and |
| | | an RNA construct described herein (e.g., comprising a RdRP- |
| | | responsive 5′-UTR, at least one gene of interest, and a |
| | | 3′UTR) is encoded (e.g., transcribed and/or translated) by a |
| | | DNA molecule, and both the protein and the DNA molecule |
| | | are delivered to a cell. |
| Protein | RNA | In some embodiments, the RdRP is provided as a protein, and |
| | | an RNA construct described herein (e.g., comprising a RdRP- |
| | | responsive 5′-UTR, at least one gene of interest, and a |
| | | 3′UTR) and both the protein and the RNA construct are |
| | | delivered to a cell. |

DNA-based delivery to a cell or a population of cells of an RdRP, a helicase, and/or an RNA construct described herein may be performed using any method known in the art (e.g., via electroporation, calcium phosphate transfection, using cationic liposomes, using polymeric nanoparticles, or a combination thereof). In some embodiments, a vector (e.g., a viral vector, a non-viral vector or a plasmid) comprising DNA encoding an RdRP, a helicase, and/or an RNA construct described herein is delivered to a cell. In some embodiments, a vector can comprise a sequence that encodes a RdRP, a helicase, and/or an RNA construct described herein. In some embodiments, the sequence that encodes a RdRP, a helicase, and/or an RNA construct is codon-optimized.

In some embodiments, a DNA molecule (e.g., vector) comprising a sequence that encodes any of the proteins or RNA constructs described herein is provided. For example, the DNA molecule may encode a RdRP, a helicase, and/or an RNA construct described herein comprises one or more regulatory/control elements (e.g., a promoter, an enhancer, a polyadenylation signal, a Kozak consensus sequence, an internal ribosome entry site (IRES), a 2A sequence, a splice acceptor or donor sequence). In some embodiments, the DNA molecule comprises a promoter (e.g., a constitutive promoter or an inducible promoter) operably-liked to the sequence encoding an RdRP. In some embodiments, the DNA molecule comprises a promoter (e.g., a constitutive promoter or an inducible promoter) operably-liked to the sequence encoding an RNA construct described herein. In some embodiments, the promoter is recognized by RNA polymerase I (i.e., a Pol I promoter). In some embodiments, the promoter is recognized by RNA polymerase II (i.e., a Pol II promoter). In some embodiments, the promoter is an inducible promoter. Examples of inducible promoters include, but are not limited to, steroid-inducible promoters (e.g., glucorticoid-inducible promoters or estrogen-inducible promoters), metallothionine promoter (inducible by treatment with various heavy metals), MX-1 promoter (inducible by interferon), the cumate-inducible gene switch (see, e.g., International Application Publ. No. WO 2002/088346), a tetracycline-inducible promoter, etc. In some embodiments, the promoter is a constitutive promoter. Examples of constitutive promoters include, but are not limited to, a cytomegalovirus (CMV) immediate early promoter, a viral simian virus 40 (SV40) (e.g., early or late) promoter, a Moloney murine leukemia virus (MoMLV) LTR promoter, a herpes simplex virus (HSV) (thymidine kinase) promoter, H5. P7.5, and P11 promoters from vaccinia virus, an elongation factor 1-alpha (EF1a) promoter, an early growth response 1 (EGR1) promoter, a ferritin H (FerH) promoter, a ferritin L (FerL) promoter, a glyceraldehyde 3-phosphate dehydrogenase (GAPDH) promoter, an eukaryotic translation initiation factor 4A1 (EIF4A 1) promoter, a heat shock 70 kDa protein 5 (HSPA5) promoter, a heat shock protein 90 kDa beta promoter, heat shock protein 70 kDa (HSP70) promoter, a β-kinesin (β-KIN) promoter, a ubiquitin C promoter (UBC), a phosphoglycerate kinase-1 (PGK) promoter, a cytomegalovirus enhancer/chicken β-actin (CAG) promoter, a β-actin promoter, and a d1587rev primer-binding site substituted (MND) promoter. In some embodiments, the promoter is a cell or tissue-type specific promoter that allows expression in a restricted variety of cell and tissue types, respectively.

The RNA constructs described herein can be delivered into a cell or into a population of cells by any method known in the art, including, but not limited to, microinjection, electroporation, lipid-mediated transfection, peptide-mediated delivery, or a combination thereof. In some embodiments, the RNA construct comprises single-stranded RNA. In some embodiments, the RNA construct comprises double-stranded RNA. In some embodiments, the RNA construct is in vitro transcribed. In some embodiments, the RNA construct comprises synthesized RNA. In some embodiments, the RNA construct comprises a linear nucleic acid molecule. In some embodiments, the RNA construct comprises a circularized nucleic acid molecule. Methods of circularizing RNA constructs are known in the art and include, but are not limited to chemical ligation and synthesis (e.g., using cyanogen bromide in the presence of a morpholino derivative as activator), enzymatic ligation (e.g., using T4 DNA ligase. T4 RNA ligase 1, T4 RNA ligase 2), and the PIE method (RNA cyclase ribozyme), described, for example, in Petkovic and Muller (2015) Nucleic Acids Research 43: 2454-65, incorporated herein by reference.

The cells and/or nucleic acid constructs described herein may comprise a helicase (e.g., a flavivirus helicase or a non-flavivirus helicase) or a gene encoding the helicase. In some embodiments, the helicase has at least 75%, 80%, 85%, 90%, 95%, 96%, 97%, 98%, 99%, or 100% sequence identity to a flavivirus helicase described herein (e.g., a Dengue virus NS3, WNV NS3, or SLEV NS3, e.g., as listed in any of Tables 1-5). A helicase can be delivered to the cell, for example, using any of the methods described herein (e.g., as listed in Table 10).

### Lipid Nanoparticle Methods

In some embodiments, an RNA described herein is introduced into a cell, e.g., an erythroid cell, using lipid nanoparticle (LNPs), e.g., by transfection. Thus, in some aspects, the disclosure provides a method of introducing an RNA described herein into a cell. e.g., an erythroid cell, comprising contacting the cell with the RNA and an LNP, e.g., an LNP described herein. The disclosure also provides reaction mixtures comprising a cell (e.g., an erythroid cell), an RNA described herein, and an LNP. In some embodiments, the RNA is complexed with the LNP. In embodiments, the population of cells contacted with the LNPs comprises at least 1×10⁷, 2×10⁷, 5×10⁷, 1×10⁸, 2×10⁸, 5×10⁸, 1×10⁹, 2×10⁹, or 5×10⁹, 1×10¹⁰, 2×10¹⁰, or 5×10¹⁰ cells.

An exemplary LNP comprises a cationic trialkyl lipid, a non-cationic lipid (e.g., PEG-lipid conjugate and a phospholipid), and an RNA molecule that is encapsulated within the lipid particle. In embodiments, the phospholipid comprises dipalmitoylphosphatidylcholine (DPPC), distearoylphosphatidylcholine (DSPC), or a mixture thereof. In embodiments, the PEG-lipid conjugate is selected from the group consisting of a PEG-diacylglycerol (PEG-DAG) conjugate, a PEG-dialkyloxypropyl (PEG-DAA) conjugate, a PEG-phospholipid conjugate, a PEG-ceramide (PEG-Cer) conjugate, and a mixture thereof. In embodiments, the PEG-DAA conjugate is selected from the group consisting of a PEG-didecyloxypropyl (C₁₀) conjugate, a PEG-dilauryloxypropyl (C₁₂) conjugate, a PEG-dimyristyloxypropyl (C₁₄) conjugate, a PEG-dipalmityloxypropyl (C₁₆) conjugate, a PEG-distearyloxypropyl (C₁₈) conjugate, and a mixture thereof. In embodiments, the LNP further comprises cholesterol. Additional LNPs are described, e.g., in US Pat. Pub. 20160256567, which is herein incorporated by reference in its entirety.

Another exemplary LNP can comprise a lipid having a structural Formula (I):

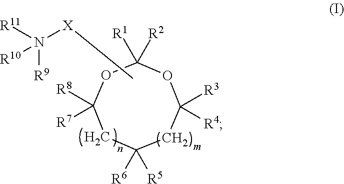

or salts thereof, wherein:
R¹, R², R³, R⁴, R⁵, R⁶, R⁷, and R⁸ are independently selected from the group consisting of hydrogen, optionally substituted C₇-C₃₀ alkyl, optionally substituted C₇-C₃₀ alkenyl and optionally substituted C₇-C₃₀ alkynyl;
provided that (a) at least two of R¹, R², R³, R⁴, R⁵, R⁶, R⁷, and Ra are not hydrogen, and (b) two of the at least two of R¹, R², R³, R⁴, R⁵, R⁶, R⁷, and R⁸ that are not hydrogen are present in a 1, 3 arrangement, a 1, 4 arrangement or a 1, 5 arrangement with respect to each other;
X is selected from the group consisting of C₁-C₆ alkyl, C₂-C₆ alkenyl and C₂-C₆ alkynyl;
R⁹, R¹⁰, and R¹¹ are independently selected from the group consisting of hydrogen, optionally substituted C₁-C₇ alkyl, optionally substituted C₂-C₇ alkenyl and optionally substituted C₂-C₇ alkynyl, provided that one of R⁹, R¹⁰, and R¹¹ may be absent; and
n and m are each independently 0 or 1.
For instance, the lipid can comprise one of the following structures:

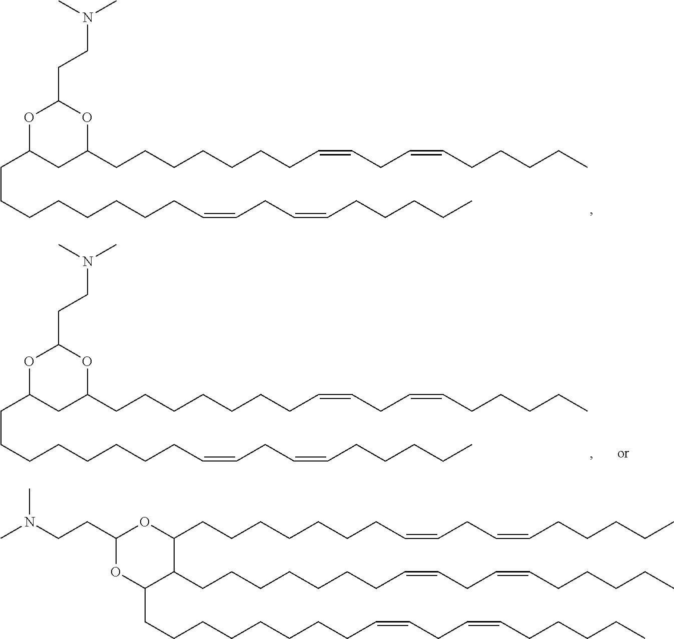

In embodiments, the LNP further comprises a non-cationic lipid such as a phospholipid, cholesterol, or a mixture of a phospholipid and cholesterol. In embodiments, the phospholipid comprises dipalmitoylphosphatidylcholine (DPPC), distearoylphosphatidylcholine (DSPC), or a mixture thereof. Additional LNPs are described, e.g., in US Pat. Pub. 20130064894, which is herein incorporated by reference in its entirety.

Another exemplary LNP comprises: (a) a nucleic acid, e.g., mRNA; (b) a cationic lipid comprising from 50 mol % to 65 mol % (e.g., 52 mol % to 62 mol %) of the total lipid present in the particle; (c) a non-cationic lipid comprising a mixture of a phospholipid and cholesterol or a derivative thereof, wherein the phospholipid comprises from 4 mol % to 10 mol % of the total lipid present in the particle and the cholesterol or derivative thereof comprises from 30 mol % to 40 mol % of the total lipid present in the particle; and (d) a conjugated lipid that inhibits aggregation of particles comprising from 0.5 mol % to 2 mol % of the total lipid present in the particle. In embodiments, the phospholipid comprises dipalmitoylphosphatidylcholine (DPPC), distearoylphosphatidylcholine (DSPC), or a mixture thereof. In embodiments, the conjugated lipid that inhibits aggregation of particles comprises a polyethyleneglycol (PEG)-lipid conjugate. In embodiments, the PEG-lipid conjugate comprises a PEG-diacylglycerol (PEG-DAG) conjugate, a PEG-dialkyloxypropyl (PEG-DAA) conjugate, or a mixture thereof. In embodiments, the PEG-DAA conjugate comprises a PEG-dimyristyloxypropyl (PEG-DMA) conjugate, a PEG-distearyloxypropyl (PEG-DSA) conjugate, or a mixture thereof. Additional LNPs are described, e.g., in U.S. Pat. No. 8,058,069, which is herein incorporated by reference in its entirety.

### Exemplary Cell Types

A nucleic acid construct (e.g., an RNA construct comprising a flavivirus 5′ UTR) as described herein can be introduced to a cell, which can in turn produce an exogenous product (e.g., an exogenous polypeptide, protein, or functional RNA) encoded by the nucleic acid construct. In some embodiments, the cell may be used to deliver the exogenous product (e.g., a therapeutic agent) into a particular region or regions within the subject (e.g., a desired target organ, site of disease, tissue, cell type, or cell). In some embodiments, the cell is a mammalian cell (e.g., a human cell, e.g., an erythroid cell, e.g., an erythroid cell precursor). In some embodiments, the cell is a plant cell, bacterial cell, fungal cell, a protozoan cell, or insect cell. The cell may be a nucleated cell (e.g., an erythroid cell precursor as described herein) or an enucleated cell (e.g., an enucleated erythroid cell, e.g., as described herein). Other exemplary cell types contemplated for use with the nucleic acid constructs herein, e.g., according to the methods herein, include mesenchymal cells, circulatory cells, stem cells (e.g., hematopoietic stem cells, mesenchymal stem cells, and neural stem cells), tumor cells, tumor-derived cells, immortalized cells, and immune cells (e.g., T cells, B cells, and antigen-presenting cells). The cell may be heterologous to the subject to which it will be administered (e.g., a cell from a cell culture or a cell obtained from a host, e.g., a universal donor) or an autologous cell obtained from or derived from the subject.

### Enucleated Erythroid Cells

In some embodiments, the erythroid cells described herein have one or more (e.g., 2, 3, 4, or more) physical characteristics described herein, e.g., osmotic fragility, cell size, hemoglobin concentration, or phosphatidylserine content. While not wishing to be bound by theory, in some embodiments an enucleated erythroid cell that expresses an exogenous protein has physical characteristics that resemble a wild-type, untreated erythroid cell. In contrast, a hypotonically loaded erythroid cell sometimes displays aberrant physical characteristics such as increased osmotic fragility, altered cell size, reduced hemoglobin concentration, or increased phosphatidylserine levels on the outer leaflet of the cell membrane.

In some embodiments, the enucleated erythroid cell comprises an exogenous protein that was encoded by an exogenous nucleic acid that was not retained by the cell, has not been purified, or has not existed fully outside an erythroid cell. In some embodiments, the erythroid cell is in a composition that lacks a stabilizer.

Osmotic Fragility

In some embodiments, the enucleated erythroid cell exhibits substantially the same osmotic membrane fragility as an isolated, uncultured erythroid cell that does not comprise an exogenous polypeptide. In some embodiments, the population of enucleated erythroid cells has an osmotic fragility of less than 50% cell lysis at 0.3%, 0.35%, 0.4%, 0.45%, or 0.5% NaCl. Osmotic fragility can be assayed using the method of Example 59 of WO2015/073587, which is herein incorporated by reference in its entirety.

Cell Size

In some embodiments, the enucleated erythroid cell has approximately the diameter or volume as a wild-type, untreated erythroid cell.

In some embodiments, the population of erythroid cells has an average diameter of about 4, 5, 6, 7, or 8 microns, and optionally the standard deviation of the population is less than 1, 2, or 3 microns. In some embodiments, the one or more erythroid cell has a diameter of about 4-8, 5-7, or about 6 microns. In some embodiments, the diameter of the erythroid cell is less than about 1 micron, larger than about 20 microns, between about 1 micron and about 20 microns, between about 2 microns and about 20 microns, between about 3 microns and about 20 microns, between about 4 microns and about 20 microns, between about 5 microns and about 20 microns, between about 6 microns and about 20 microns, between about 5 microns and about 15 microns or between about 10 microns and about 30 microns. Cell diameter is measured, in some embodiments, using an Advia 120 hematology system.

In some embodiment the volume of the mean corpuscular volume of the erythroid cells is greater than 10 fL, 20 fL, 30 fL, 40 fL, 50 fL, 60 fL, 70 fL, 80 fL, 90 fL, 100 fL, 110 fL, 120 fL, 130 fL, 140 fL, 150 fL, or greater than 150 fL. In one embodiment the mean corpuscular volume of the erythroid cells is less than 30 fL, 40 fL, 50 fL, 60 fL, 70 fL, 80 fL, 90 fL, 100 fL, 110 fL, 120 fL, 130 fL, 140 fL, 150 fL, 160 fL, 170 fL, 180 fL, 190 fL, 200 fL, or less than 200 fL. In one embodiment the mean corpuscular volume of the erythroid cells is between 80-100, 100-200, 200-300, 300-400, or 400-500 femtoliters (fL). In some embodiments, a population of erythroid cells has a mean corpuscular volume set out in this paragraph and the standard deviation of the population is less than 50, 40, 30, 20, 10, 5, or 2 fL. The mean corpuscular volume is measured, in some embodiments, using a hematological analysis instrument, e.g., a Coulter counter.

Hemoglobin Concentration

In some embodiments, the enucleated erythroid cell has a hemoglobin content similar to a wild-type, untreated erythroid cell. In some embodiments, the erythroid cells comprise greater than 1%, 2%, 3%, 4%, 5%, 6%, 7%, 8%, 9% or greater than 10% fetal hemoglobin. In some embodiments, the erythroid cells comprise at least about 20, 22, 24, 26, 28, or 30 pg, and optionally up to about 30 pg, of total hemoglobin. Hemoglobin levels are determined, in some embodiments, using the Drabkin's reagent method of Example 33 of WO2015/073587, which is herein incorporated by reference in its entirety.

Phosphatidylserine Content

In some embodiments, the enucleated erythroid cell has approximately the same phosphatidylserine content on the outer leaflet of its cell membrane as a wild-type, untreated erythroid cell. Phosphatidylserine is predominantly on the inner leaflet of the cell membrane of wild-type, untreated erythroid cells, and hypotonic loading can cause the phosphatidylserine to distribute to the outer leaflet where it can trigger an immune response. In some embodiments, the population of erythroid cells comprises less than about 30, 25, 20, 15, 10, 9, 8, 6, 5, 4, 3, 2, or 1% of cells that are positive for Annexin V staining. Phosphatidylserine exposure is assessed, in some embodiments, by staining for Annexin-V-FITC, which binds preferentially to PS, and measuring FITC fluorescence by flow cytometry, e.g., using the method of Example 54 of International Application WO2015/073587, which is herein incorporated by reference in its entirety.

Other Characteristics

In some embodiments, an erythroid cell (e.g., enucleated erythroid cell), or a population of erythroid cells comprises one or more of (e.g., all of) endogenous GPA (C235a), transferrin receptor (CD71), Band 3 (CD233), or integrin alpha4 (C49d). These proteins can be measured, e.g., as described in Example 10 of International Application Publication No. WO2018/009838, which is herein incorporated by reference in its entirety. The percentage of GPA-positive cells and Band 3-positive cells typically increases during maturation of an erythroid cell, and the percentage of integrin alpha4-positive typically remains high throughout maturation.

In some embodiments, the population of erythroid cells comprises at least about 50%, 60%, 70%, 80%, 90%, or 95% (and optionally up to 90 or 100%) of cells that are positive for GPA. The presence of GPA is detected, in some embodiments, using FACS.

In some embodiments, the population of enucleated erythroid cells comprises at least about 50%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% GPA⁺ (i.e., CD235a⁺) cells. In some embodiments, the population of enucleated erythroid cells comprises between about 50% and about 100% (e.g., from about 60% and about 100%, from about 65% and about 100%, from about 70% and about 100%, from about 75% to about 100%, from about 80% to about 100%, from about 85% to about 100%, from about 90% to about 100%, from about 95% to about 100%, from about 75% to about 99%, from about 80% to about 99%, from about 85% to about 99%, from about 90% to about 99%, from about 95% to about 99%, from about 75% to about 95%, from about 80% to about 95%, from about 85% to about 95%, from about 90% to about 95%, from about 95% to about 98%) GPA⁺ cells. The presence of GPA is detected, in some embodiments, using FACS.

In some embodiments, the population of enucleated erythroid cells comprises at least about 50%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% CD71⁺ cells. In some embodiments, the population of enucleated erythroid cells comprises between about 70% and about 100% (e.g., from about 75% to about 100%, from about 80% to about 100%, from about 85% to about 100%, from about 90% to about 100%, from about 95% to about 100%, from about 75% to about 99%, from about 80% to about 99%, from about 85% to about 99%, from about 90% to about 99%, from about 95% to about 99%, from about 75% to about 95%, from about 80% to about 95%, from about 85% to about 95%, from about 90% to about 95%, from about 95% to about 98%) CD71⁺ cells. The presence of CD71 (transferrin receptor) is detected, in some embodiments, using FACS.

In some embodiments, the population of enucleated erythroid cells comprises at least about 50%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% CD233⁺ cells. In some embodiments, the population of enucleated erythroid cells comprises between about 70% and about 100% (e.g., from about 75% to about 100%, from about 80% to about 100%, from about 85% to about 100%, from about 90% to about 100%, from about 95% to about 100%, from about 75% to about 99%, from about 80% to about 99%, from about 85% to about 99%, from about 90% to about 99%, from about 95% to about 99%, from about 75% to about 95%, from about 80% to about 95%, from about 85% to about 95%, from about 90% to about 95%, from about 95% to about 98%) CD233⁺ cells. The presence of CD233 (Band 3) is detected, in some embodiments, using FACS.

In some embodiments, the population of enucleated erythroid cells comprises at least about 50%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% CD47⁺ cells. In some embodiments, the population of enucleated erythroid cells comprises between about 70% and about 100% (e.g., from about 75% to about 100%, from about 80% to about 100%, from about 85% to about 100%, from about 90% to about 100%, from about 95% to about 100%, from about 75% to about 99%, from about 80% to about 99%, from about 85% to about 99%, from about 90% to about 99%, from about 95% to about 99%, from about 75% to about 95%, from about 80% to about 95%, from about 85% to about 95%, from about 90% to about 95%, from about 95% to about 98%) CD47⁺ cells. The presence of CD47 (integrin associate protein) is detected, in some embodiments, using FACS.

In some embodiments, the population of enucleated erythroid cells comprises at least about 50%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% CD36⁻ (CD36-negative) cells.

In some embodiments, the population of enucleated erythroid cells comprises between about 70% and about 100% (e.g., from about 75% to about 100%, from about 80% to about 100%, from about 85% to about 100%, from about 90% to about 100%, from about 95% to about 100%, from about 75% to about 99%, from about 80% to about 99%, from about 85% to about 99%, from about 90% to about 99%, from about 95% to about 99%, from about 75% to about 95%, from about 80% to about 95%, from about 85% to about 95%, from about 90% to about 95%, from about 95% to about 98%) CD36⁻ (CD36-negative) cells. The presence of CD36 is detected, in some embodiments, using FACS.

In some embodiments, the population of enucleated erythroid cells comprises at least about 50%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% CD34⁻ (CD34-negative) cells. In some embodiments, the population of enucleated erythroid cells comprises between about 70% and about 100% (e.g., from about 75% to about 100%, from about 80% to about 100%, from about 85% to about 100%, from about 90% to about 100%, from about 95% to about 100%, from about 75% to about 99%, from about 80% to about 99%, from about 85% to about 99%, from about 90% to about 99%, from about 95% to about 99%, from about 75% to about 95%, from about 80% to about 95%, from about 85% to about 95%, from about 90% to about 95%, from about 95% to about 98%) CD34⁻ (CD34-negative) cells. The presence of CD34 is detected, in some embodiments, using FACS.

In some embodiments, the population of enucleated erythroid cells comprises at least about 50%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% CD235a⁺/CD47⁺/CD233⁺ cells. In some embodiments, the population of enucleated erythroid cells comprises between about 70% and about 100% (e.g., from about 75% to about 100%, from about 80% to about 100%, from about 85% to about 100%, from about 90% to about 100%, from about 95% to about 100%, from about 75% to about 99%, from about 80% to about 99%, from about 85% to about 99%, from about 90% to about 99%, from about 95% to about 99%, from about 75% to about 95%, from about 80% to about 95%, from about 85% to about 95%, from about 90% to about 95%, from about 95% to about 98%) CD235a⁺/CD47⁺/CD233⁺ cells.

In some embodiments, the population of enucleated erythroid cells comprises at least about 50%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% CD235a⁺/CD47⁺/CD233+/CD34⁻/CD36⁻ cells. In some embodiments, the population of enucleated erythroid cells comprises between about 70% and about 100% (e.g., from about 75% to about 100%, from about 80% to about 100%, from about 85% to about 100%, from about 90% to about 100%, from about 95% to about 100%, from about 75% to about 99%, from about 80% to about 99%, from about 85% to about 99%, from about 90% to about 99%, from about 95% to about 99%, from about 75% to about 95%, from about 80% to about 95%, from about 85% to about 95%, from about 90% to about 95%, from about 95% to about 98%) CD235a⁺/CD47⁺/CD233⁺/CD34⁻/CD36⁻ cells.

In some embodiments, a population of enucleated erythroid cells comprising erythroid cells comprises less than about 10%, 9%, 8%, 7%, 6%, 5%, 4%, 3%, 2%, or 1% echinocytes.

In some embodiments, a population of enucleated erythroid cells comprises less than about 10%, 9%, 8%, 7%, 6%, 5%, 4%, 3%, 2%, or 1% pyrenocytes.

In some embodiments, the erythroid cells have a half-life of at least 0.5, 1, 2, 7, 14, 30, 45, or 90 days in a subject.

In some embodiments, an erythroid cell is enucleated, e.g., a population of cells comprising erythroid cells used as a therapeutic preparation described herein is greater than 50%, 60%, 70%, 80%, 90% enucleated. In some embodiments, a cell. e.g., an erythroid cell, contains a nucleus that is non-functional, e.g., has been inactivated.

Universal Donor Erythroid Cells

In some embodiments, erythroid cells described herein are autologous or allogeneic to the subject to which the cells will be administered. For example, erythroid cells allogeneic to the subject include one or more of blood type specific erythroid cells (e.g., the cells can be of the same blood type as the subject) or one or more universal donor erythroid cells. In some embodiments, the enucleated erythroid cells described herein have reduced immunogenicity compared to a reference cell. e.g., have lowered levels of one or more blood group antigens.

Where allogeneic cells are used for transfusion, a compatible ABO blood group can be chosen to prevent an acute intravascular hemolytic transfusion reaction. The ABO blood types are defined based on the presence or absence of the blood type antigens A and B, monosaccharide carbohydrate structures that are found at the termini of oligosaccharide chains associated with glycoproteins and glycolipids on the surface of the erythrocytes (reviewed in Liu et al., Nat. Biotech. 25:454-464 (2007)). Because group O erythrocytes contain neither A nor B antigens, they can be safely transfused into recipients of any ABO blood group. e.g., group A. B, AB, or O recipients. Group O erythrocytes are considered universal and may be used in all blood transfusions. Thus, in some embodiments, an erythroid cell described herein is type O. In contrast, group A erythroid cells may be given to group A and AB recipients, group B erythroid cells may be given to group B and AB recipients, and group AB erythroid cells may be given to AB recipients.

In some instances, it may be beneficial to convert a non-group O erythroid cell to a universal blood type. Enzymatic removal of the immunodominant monosaccharides on the surface of group A and group B erythrocytes may be used to generate a population of group O-like erythroid cells (See. e.g., Liu et al., Nat. Biotech. 25:454-464 (2007)). Group B erythroid cells may be converted using an α-galactosidase derived from green coffee beans. Alternatively or in addition, α-N-acetylgalactosaminidase and α-galactosidase enzymatic activities derived from E. meningosepticum bacteria may be used to respectively remove the immunodominant A and B antigens (Liu et al., Nat. Biotech. 25:454-464 (2007)), if present on the erythroid cells. In one example, packed erythroid cells isolated as described herein, are incubated in 200 mM glycine (pH 6.8) and 3 mM NaCl in the presence of either α-N-acetylgalactosaminidase and α-galactosidase (about 300 μg/ml packed erythroid cells) for 60 min at 26° C. After treatment, the erythroid cells are washed by 3-4 rinses in saline with centrifugation and ABO-typed according to standard blood banking techniques.

While the ABO blood group system is the most important in transfusion and transplantation, in some embodiments it can be useful to match other blood groups between the erythroid cells to be administered and the recipient, or to select or make erythroid cells that are universal for one or more other (e.g., minor) blood groups. A second blood group is the Rh system, wherein an individual can be Rh+ or Rh−. Thus, in some embodiments, an erythroid cell described herein is Rh−. In some embodiments, the erythroid cell is Type O and Rh−.

In some embodiments, an erythroid cell described herein is negative for one or more minor blood group antigens, e.g., Le(a-b-) (for Lewis antigen system), Fy(a-b-) (for Duffy system), Jk(a-b-) (for Kidd system), M-N- (for MNS system), K-k- (for Kell system), Lu(a-b-) (for Lutheran system), and H-antigen negative (Bombay phenotype), or any combination thereof. In some embodiments, the erythroid cell is also Type 0 and/or Rh-. Minor blood groups are described. e.g., in Agarwal et al “Blood group phenotype frequencies in blood donors from a tertiary care hospital in north India” Blood Res. 2013 March; 48(1): 51-54 and Mitra et al “Blood groups systems” Indian J Anaesth. 2014 September-October; 58(5): 524-528, each of which is incorporated herein by reference in its entirety.

### Methods of Manufacturing Enucleated Erythroid Cells

Methods of manufacturing enucleated erythroid cells comprising (e.g., expressing) an exogenous agent (e.g., a polypeptide) are described. e.g., in WO2015/073587 and WO2015/153102, each of which is incorporated by reference in its entirety.

In some embodiments, hematopoietic progenitor cells, e.g., CD34+ hematopoietic progenitor cells or erythroid cell precursors, are contacted with a nucleic acid or nucleic acids encoding one or more exogenous polypeptides, and the cells are allowed to expand and differentiate in culture.

In some embodiments, the two or more polypeptides are encoded in a single nucleic acid, e.g., a single vector. In embodiments, the single vector has a separate promoter for each gene, has two proteins that are initially transcribed into a single polypeptide having a protease cleavage site in the middle, so that subsequent proteolytic processing yields two proteins, or any other suitable configuration. In some embodiments, the two or more polypeptides are encoded in two or more nucleic acids, e.g., each vector encodes one of the polypeptides.

The nucleic acid may be, e.g., DNA or RNA. A number of viruses may be used as gene transfer vehicles including retroviruses, Moloney murine leukemia virus (MMLV), adenovirus, adeno-associated virus (AAV), herpes simplex virus (HSV), lentiviruses such as human immunodeficiency virus 1 (HIV 1), and spumaviruses such as foamy viruses, for example.

In some embodiments, e.g., in combination with the RNA-based methods described herein, the cells are produced using conjugation, e.g., sortagging, e.g., as described in WO2014/183071 or WO2014/183066, each of which is incorporated by reference in its entirety.

In some embodiments, the erythroid cells are expanded at least 1000, 2000, 5000, 10,000, 20,000, 50,000, or 100,000 fold (and optionally up to 100,000, 200,000, or 500,000 fold). Number of cells is measured, in some embodiments, using an automated cell counter.

In some embodiments, the population of erythroid cells comprises at least 30, 35, 40, 45, 50, 55, 60, 65, 70, 75, 80, 85, 90, 95, or 98% (and optionally up to about 80, 90, or 100%) enucleated erythroid cells. In some embodiments, the population of erythroid cells contains less than 1% live nucleated cells, e.g., contains no detectable live nucleated cells. Enucleation is measured, in some embodiments, by FACS using a nuclear stain. In some embodiments, at least 30, 35, 40, 45, 50, 55, 60, 65, 70, 75, or 80% (and optionally up to about 70, 80, 90, or 100%) of erythroid cells in the population comprise one or more (e.g., 2, 3, 4 or more) of the exogenous polypeptides. Expression of the polypeptides is measured, in some embodiments, by erythroid cells using labeled antibodies against the polypeptides. In some embodiments, at least 30, 35, 40, 45, 50, 55, 60, 65, 70, 75, or 80% (and optionally up to about 70, 80, 90, or 100%) of erythroid cells in the population are enucleated and comprise one or more (e.g., 2, 3, 4, or more) of the exogenous polypeptides. In some embodiments, the population of erythroid cells comprises about 1×10⁹-2×10⁹, 2×10⁹-5×10⁹, 5×10⁹-1×10¹⁰, 1×10¹⁰-2×10¹⁰, 2×10¹⁰-5×10¹⁰, 5×10¹¹-1×10¹¹, 1×10¹¹-2×10¹¹, 2×10¹¹-5×10¹¹, 5×10¹¹-×10¹², 1×10¹²-2×10¹², 2×10¹²-5×10¹², or 5×10¹²-×10¹³ cells.

Cells Encapsulated in a Membrane

In some embodiments, a cell (e.g., an enucleated erythroid cell) or other vehicles described herein are encapsulated in a membrane, e.g., semi-permeable membrane. In embodiments, the membrane comprises a polysaccharide, e.g., an anionic polysaccharide alginate. In embodiments, the semipermeable membrane does not allow cells to pass through, but allows passage of small molecules or macromolecules. e.g., metabolites, proteins, or DNA. In embodiments, the membrane is one described in Lienert et al., “Synthetic biology in mammalian cells: next generation research tools and therapeutics” Nature Reviews Molecular Cell Biology 15, 95-107 (2014), incorporated herein by reference in its entirety. While not wishing to be bound by theory, in some embodiments, the membrane shields the cells from the immune system and/or keeps a plurality of cells in proximity, facilitating interaction with each other or each other's products.

### Uses of Compositions

Compositions described herein can be used in a variety of contexts, such as for therapeutic administration to a subject. For example, a nucleic acid construct described herein may encode the therapeutic agent. The nucleic acid construct can be introduced to a cell (e.g., an erythroid cell), which can then be administered to the subject. Non-limiting examples of uses of compositions described herein are described below.

### Expressing Therapeutic Agents

In some embodiments, a composition comprising a nucleic acid construct as described herein can be introduced into a cell (e.g., an erythroid cell. e.g., an erythroid cell precursor). The cell may further comprise an RdRP capable of replicating the nucleic acid construct. The cell may further produce a therapeutic agent (e.g., a therapeutic protein or a therapeutic functional RNA) encoded by the nucleic acid construct. In embodiments, an RNA construct comprising a flavivirus 5′ UTR and a nucleic acid sequence encoding a therapeutic protein is introduced into an erythroid cell precursor. In embodiments, an RdRP in the erythroid cell precursor directs replication of the RNA construct, e.g., by directing synthesis of the reverse complement of the RNA construct. In embodiments, the erythroid cell precursor expresses the encoded therapeutic protein. In embodiments, the therapeutic protein is secreted by the cell, e.g., erythroid cell precursor. In embodiments, the therapeutic protein is a membrane-bound protein displayed on the cell surface of the erythroid cell precursor.

In some embodiments, the exogenous agent can be used to engineer the erythroid cell precursor, e.g., to alter the behavior, morphology, activity, and/or other property of the erythroid cell precursor. For example, the therapeutic agent may be a signaling factor that modulates (e.g., increases or decreases) proliferation, differentiation, motility, cell death, gene expression (e.g., of other signaling factors), autophagy, tissue homing, and/or cell signaling by the erythroid cell precursor. In embodiments, the erythroid cell precursor may express a plurality of therapeutic agents (e.g., encoded by one or more nucleic acid constructs, e.g., RNA constructs, described herein), one or more of which serves to engineer a property of the erythroid cell precursor, and one or more of which is intended for delivery to a cell, tissue, or organ of the subject.

### Vaccination

In some embodiments, a nucleic acid construct described herein comprises a nucleic acid sequence encoding a vaccine (e.g., an RNA vaccine or a polypeptide vaccine). In some embodiments, the nucleic acid construct is an RNA construct encoding an antigenic RNA or an antigenic polypeptide. In some embodiments, the antigenic RNA or polypeptide is expressed by a cell. e.g., an erythroid cell to which the construct is introduced, e.g., according to the methods herein. In some embodiments, the antigenic RNA or polypeptide is secreted by the cell or is displayed by the cell, thereby exposing it to the subject's immune system. In some embodiments, the antigenic RNA or polypeptide induces an immune response in the subject.

### Gene Therapy

A composition described herein can be provided to a subject for use in gene therapy, e.g., for ectopic expression of a gene that is not expressed in the subject, or which is expressed at levels lower than for a healthy control subject (e.g., a gene for which functional copies are not expressed by the subject), and/or a mutant gene, e.g., a gene encoding a protein with reduced activity compared to a wild-type protein. In some embodiments, the subject does not natively express the gene, e.g., due to downregulation of expression of the gene or due to the subject comprising one or more nonfunctional copies of the gene in the subject's genome. In some embodiments, the ectopic expression of the gene is used for gene replacement therapy. In embodiments, the gene being replaced using the composition described herein encodes a protein, e.g., an enzyme. In embodiments, the protein is cystic fibrosis transmembrane conductance regulator (CFTR) and the subject has cystic fibrosis. In embodiments, the protein is SMA1 and the subject has spinal muscular atrophy.

In some embodiments, the disease is a genetic disease. In other embodiments, the disease is an infectious disease. In some embodiments, the subject has an immunodeficiency (e.g., adenosine deaminase (ADA) deficiency, sickle cell anemia, thalassemia, hemophilia, and familial hypercholesterolemia). In embodiments, the subject has cancer, AIDS, heart disease, or another disease featuring reduced expression of a gene.

### Reprogramming Cells

A nucleic acid construct described herein can comprise one or more exogenous nucleic acid sequences, each encoding one or more factors for cellular reprogramming. In some embodiments, a nucleic acid construct described herein encodes one or more factors for converting a somatic cell (e.g., a fibroblast) into a pluripotent cell (e.g., an induced pluripotent stem (iPS) cell). In some embodiments, the nucleic acid construct encodes one or more of Oct4, Sox2. Klf4, and c-Myc (e.g., human Oct4, Sox2, Klf4, and c-Myc). A cell as described herein may comprise one or more such nucleic acid constructs. In some embodiments, a nucleic acid construct described herein comprises an exogenous nucleic acid sequence encoding a signaling factor that can induce reprogramming in another cell (e.g., a cell to be converted into a different cell type), e.g., wherein the signaling factor is secreted or displayed on the surface of the cell (e.g., an erythroid cell) comprising the nucleic acid construct. In some embodiments, a nucleic acid construct described herein comprises an exogenous nucleic acid sequence encoding a factor that promotes immortalization of a cell (e.g., telomerase or a viral protein that inhibits cell senescence. e.g., adenovirus type 5 E1, SV-40 large T-antigen, and/or human papillomavirus E6 and/or E7).

### Immune Response

A nucleic acid construct described herein can be introduced into an immune cell (e.g., a T cell, B cell, or antigen-presenting cell) and used to express an exogenous gene. In some embodiments, the nucleic acid construct comprises an exogenous nucleic acid sequence encoding, e.g., an antibody molecule, or an antigen-binding fragment thereof, or a T cell receptor, or an antigen-binding fragment thereof.

In some embodiments, a cell as described herein comprises a nucleic acid construct described herein, wherein the nucleic acid construct comprises an exogenous nucleic acid sequence encoding an anti-inflammatory protein. In embodiments, the cell expresses the anti-inflammatory protein, thereby suppressing an immune response, e.g., in the surrounding tissue. In embodiments, the cell expresses the anti-inflammatory protein in a draining lymph node.

In some embodiments, a cell as described herein comprises a nucleic acid construct described herein, wherein the nucleic acid construct comprises an exogenous nucleic acid sequence encoding a protein that promotes an immune response (e.g., a checkpoint modulator). In embodiments, the cell expresses the protein that promotes an immune response, thereby promoting an immune response, e.g., in the surrounding tissue. In embodiments, the surrounding tissue comprises cancer cells. In embodiments, the cell is used to treat cancer in a subject by delivering a protein that promotes an immune response (e.g., a checkpoint modulator) to the cancer.

### Methods of Treatment

A cell expressing an exogenous gene as described herein can be administered to a subject to treat a disease or disorder. In some embodiments, the cell is further incubated after introduction of a nucleic acid construct described herein, e.g., to induce enucleation of the cell. In some embodiments, cells described herein are administered to a subject, e.g., a mammal. e.g., a human. Exemplary mammals that can be treated include without limitation, humans, domestic animals (e.g., dogs, cats and the like), farm animals (e.g., cows, sheep, pigs, horses and the like) and laboratory animals (e.g., monkey, rats, mice, rabbits, guinea pigs and the like). The methods described herein are applicable to both human therapy and veterinary applications.

In some embodiments, a cell is used to deliver an exogenous gene product to a particular. e.g., therapeutically important area of the body (e.g., a cell type, tissue, or organ). In other embodiments, a cell is used to deliver an exogenous gene product systemically.

In some embodiments, the cell is an erythroid cell, e.g., an enucleated erythroid cell. Methods of administering enucleated erythroid cells (e.g., reticulocytes) comprising (e.g., expressing) an exogenous agent (e.g., a polypeptide) are described, e.g., in WO2015/073587 and WO2015/153102, each of which is incorporated by reference in its entirety.

In some embodiments, the erythroid cells are administered to a patient every 1, 2, 3, 4, 5, or 6 months.

In some embodiments, a dose of erythroid cells comprises about 1×10⁹-2×10⁹, 2×10⁹-5×10⁹, 5×10⁹-1×10¹⁰, 1×10¹⁰-2×10¹⁰, 2×10¹⁰-5×10¹⁰, 5×10¹⁰-×10¹¹, 1×10¹¹-2×10¹¹, 2×10¹¹-5×10¹¹, 5×10¹¹-1×10¹², 1×10¹²-2×10¹², 2×10¹²-5×10¹², or 5×10¹²-1×10¹³ cells.

In some embodiments, the erythroid cells are administered to a patient in a dosing regimen (dose and periodicity of administration) sufficient to maintain function of the administered erythroid cells in the bloodstream of the patient over a period of 2 weeks to a year. e.g., one month to one year or longer, e.g., at least 2 weeks, 4 weeks, 6 weeks, 8 weeks, 3 months, 6 months, a year, 2 years.

In some embodiments, the erythroid cell is modified to express 4-1BBL, anti-CD20, TRAIL, anti-PD-L1, or asparaginase using a nucleic acid construct as described herein, and can be used to treat cancer in a subject in need thereof. In some embodiments, the erythroid cell expresses phenylalanine ammonia lyase (PAL) and can be used to reduce phenylalanine levels in a subject in need thereof. e.g., to treat phenylketonuria (PKU) or hyperphenylalaninemia. In some embodiments, the erythroid cells express a clotting factor, e.g., clotting factor X, and are used to treat a clotting disease in a subject in need thereof.

### Tables

TABLE 11
Exemplary exogenous polypeptides for expression in erythroid cells

| Protein name | Sequence | SEQ ID NO: |
|---|---|---|
| 4-1BBL | ACPWAVSGARASPGSAASPRLREGPELSPDDPAGLLDL | 44 |
| | RQGMFAQINAQNVLLIDGPLSWYSDPGLACIVSLTGGL | |
| | SYKEDTKELYVAKAGVYYVFFQLELRRVVAGEGSGSV | |
| | SLALHLQPLRSAAGAAALALTVDLPPASSEARNSAFGF | |
| | QGRLLHLSAGQRLGVHLHTEARARHAWQLTQGATVL | |
| | GLFRVTPEIPAGLPSPRSE | |
| Anti-CD20 | Rituximab heavy chain chimeric: | 45, 46 |
| | QVQLQQPGAELVKPGASVKMSCKASGYTFTSYNMHW | |
| | VKQTPGRGLEWIGAIYPGNGDTSYNQKFKGKATLTAD | |
| | KSSSTAYMQLSSLTSEDSAVYYCARSTYYGGDWYFNV | |
| | WGAGTTVTVSAASTKGPSVFPLAPSSKSTSGGTAALGC | |
| | LVKDYFPEPVTVSWNSGALTSGVHTFPAVLQSSGLYSL | |
| | SSVVTVPSSSLGTQTYICNVNHKPSNTKVDKKAEPKSC | |
| | DKTHTCPPCPAPELLGGPSVFLFPPKPKDTLMISRTPEV | |
| | TCVVVDVSHEDPEVKFNWYVDGVEVHNAKTKPREEQ | |
| | YNSTYRVVSVLTVLHQDWLNGKEYKCKVSNKALPAPI | |
| | EKTISKAKGQPREPQVVTLPPSRDELTKNQVSSLTCLVK | |
| | GFYPSDIAVEWESNGQPENNYKTTPPVLDSDGSFFLYS | |
| | KLTVDKSRWQQGNVFSCSVMHEALHNHYTQKSLSLSP | |
| | GK (SEQ ID NO: 45) | |
| | Rituximab light chain chimeric: | |
| | QIVLSQSPAILSASPGEKVTMTCRASSSVSYIHWFQQKP | |
| | GSSPKPWIYATSNLASGVPVRFSGSGSGTSYSLTISRVE | |
| | AEDAATYYCQQWTSNPPTFGGGTKLEIKRTVAAPSVFI | |
| | FPPSDEQLKSGTASVVCLLNNFYPREAKVQWKVDNAL | |
| | QSGNSQESVTEQDSKDSTYSLSSTLTLSKADYEKHKVY | |
| | ACEVTHQGLSSPVTKSFNRGEC (SEQ ID NO: 46) | |
| TRAIL | Soluble TRAIL variant DR4-1 | 47-51 |
| | MAMMEVQGGPSLGQTCVLIVIFTVLLQSLCVAVTYVY | |
| | FTNELKQMQDKYSKSGIACFLKEDDSYWDPNDEESMN | |
| | SPCWQVKWQLRQUIRKMILRTSEETISTVQEKQQNISP | |
| | LVRERGPQRVAAHITGTRRRSNTLSSPNSKNEKALGRK | |
| | INSWESSRSGHSFLSNLHLRNGELVIHEKGFYYIYSQTY | |
| | FRFQEEIKENTKNDKQMVQYIYKYTSYPDPILLMKSAR | |
| | NSCWSKDAEYGLYSIYQGGIFELKENDRIFVSVTNEHLI | |
| | DMDHEASFFGAFLVG (SEQ ID NO: 47) | |
| | Soluble TRAIL variant DR4-2 | |
| | MAMMEVQGGPSLGQTCVLIVIFTVLLQSLCVAVTYVY | |
| | FTNELKQMQDKYSKSGIACFLKEDDSYWDPNDEESMN | |
| | SPCWQVKWQLRQLVRKMILRTSEETISTVQEKQQNISP | |
| | LVRERGPQRVAAFITGTRGRSNTLSSPNSKNEKALGRK | |
| | INSWESSRRGHSFLSNLHLRNGELVIHEKGFYYIYSQTY | |
| | FRFQEEIKENTKNDKQMVQYIYKYTSYPDPILLMKSAR | |
| | NSCWSKDAEYGLYSIYQGGIFELKENDRIFVSVTNEHLI | |
| | DMDHEASFFGAFLVG (SEQ ID NO: 48) | |
| | Soluble TRAIL variant DR4-3 | |
| | MAMMEVQGGPSLGQTCVLIVIFTVLLQSLCVAVTYVY | |
| | FTNELKQMQDKYSKSGIACFLKEDDSYWDPNDEESMN | |
| | SPCWQVKWQLRQLVRKMILRTSEETISTVQEKQQNISP | |
| | LVRERGPQRVAAHITGTRRRSNTLSSPNSKNEKALGIKI | |
| | NSWESSRRGHSFLSNLHLRNGELVIHEKGFYYIYSQTY | |
| | FRFQEEIKENTKNDKQMVQYIYKYTDYPDPILLMKSAR | |
| | NSCWSKDAEYGLYSIYQGGIFELKENDRIFVSVTNEHLI | |
| | DMDHEASFFGAFLVG (SEQ ID NO: 49) | |
| | Soluble TRAIL variant DR5-1 | |
| | MAMMEVQGGPSLGQTCVLIVIFTVLLQSLCVAVTYVY | |
| | FTNELKQMQDKYSKSGIACFLKEDDSYWDPNDEESMN | |
| | SPCWQVKWQLRQLVRKMILRTSEETISTVQEKQQNISP | |
| | LVRERGPQRVAAHITGTRGRSNTLSSPNSKNEKALGRK | |
| | INSWESSRSGHSFLSNLHLRNGELVIHEKGFYYIYSQTY | |
| | FRFQEEIKENTKNDKQMVQYTYKYTSYPDPILLMKSAR | |
| | NSCWSKDAENGLYSIYQGGIFELKENDRIFVSVTNEHLI | |
| | DMHHEASFFGAFLVG (SEQ ID NO: 50) | |
| | Soluble TRAIL variant DR5-2 | |
| | MAMMEVQGGPSLGQTCVLIVIFTVLLQSLCVAVTYVY | |
| | FTNELKQMQDKYSKSGIACFLKEDDSYWDPNDEESMN | |
| | SPCWQVKWQLRQLVRKMILRTSEETISTVQEKQQNISP | |
| | LVRERGPQRVAAHITGTRGRSNTLSSPNSKNEKALGRK | |
| | INSWESSRSGHSFLSNLHLRNGELVIHEKGFYYIYSQTY | |
| | FRFQERIKENTKNDKQMVQYIYKYTSYPDPILLMKSAR | |
| | NSCWSKDAEYGLYSIYQGGIFELKENDRIFVSVTNEHLI | |
| | DMHHEASFFGAFLVG (SEQ ID NO: 51) | |
| Anti-PD-L1 | VQLVESGGGLVQPGGSLRLSCAASGFTFSDSWIHWVR | 52 |
| scFv | QAPGKGLEWVAWISPYGGSTYYADSVKGRFTISADTS | |
| | KNTAYLQMNSLRAEDTAVYNCARRHWPGGFDYWGQ | |
| | GTLVTVSSGGGGSGGGGSGGGGSIQMTQSPSSLSASVG | |
| | DRVTITCRASQDVSTAVAWYQQKPGKAPKLLIYSASFL | |
| | YSGVPSRFSGSGSGTDFTLTISSLQPEDFATYYCQQYLY | |
| | HPATFGQGTKVEIK | |
| phenylalanine | MKTLSQAQSKTSSQQFSFTGNSSANVIIGNQKLTINDV | 53 |
| ammonia lyase | ARVARNGTLVSLTNNTDILQGIQASCDYINNAVESGEPI | |
| (PAL | YGVTSGFGGMANVAISREQASELQTNLVWFLKTGAGN | |
| | KLPLADVRAAMLLRANSHMRGASGIRLELIKRMEIFLN | |
| | AGVTPYVYEFGSIGASGDLVPLSYITGSLIGLDPSFKVD | |
| | FNGKEMDAPTALRQLNLSPLTLLPKEGLAMMNGTSV | |
| | MTGIAANCVYDTQILTAIAMGVHALDIQALNGTNQSF | |
| | HPFIHNSKPHPGQLWAADQMISLLASQLVRDELDGK | |
| | HDYRDHELIQDRYSLRCLPQYLGPIVDGISQIAKQIEIEI | |
| | NSVTDNPLIDVDNQASYHGGNFLGQYVGMGMDHLRY | |
| | YIGLLAKHLDVQIALLASPEFSNGLPRSLLGNRERKVN | |
| | MGLKGLQICGNSIMPLLTFYGNSIADRFPTHAEQFNQNI | |
| | NSQGYTSATLARRSVDIFQNYVAIALMFGVQAVDLRT | |
| | YKKTGHYDARACLSPATERLYSAVRHVVGQKPTSDRPYI | |
| | WNDNEQGLDEHIARISADIAAGGVIVQAVQDILPCLH | |
| Y vb | MADKLPNIVILATGGTIAGSAATGTQTTGYKAGALGV | 54 |
| Asparaginase | DTLINAVPEVKKLANVKGEQESNMASENNFFGDVVLK | |
| | LSQRVNELLARDDVDGVVITHGTDTVEESAYFLHLTV | |
| | KSDKPVVFVAAMRPATAISADGPMNLLEAVRVAGDK | |
| | QSRGRGVMVVLNDRIGSARYITKTNASTLDTFKANEE | |
| | GYLGVIIGNRIYYQNRIDKLHTTRSVETWRGLTSLPKVD | |
| | ILYGYQDDPEYLYDAAIQHGVKGIVYAGMGAGSVSVR | |
| | GIAGMRKAMEKGVVVIRSTRTGNGIVPPDEELPGLVSD | |
| | SLNPAHARILLMLALTRTSDPKVIQEYFHTY | |
| Anti-integrin | Heavy chain variable region: | 55, 56 |
| α4β7 antibody | QVQLVQSGAEVKKPGASVKVSCKGSGYTFTSYWMHW | |
| | VRQAPGQRLEWIGEIDPSESNTNYNQKFKGRVTLTVDI | |
| | SASTAYMELSSLRSEDTAVYYCARGGYDGWDYAIDY | |
| | WGQGTLVITVSS (SEQ ID NO: 55) | |
| | Light chain variable region: | |
| | DVVMTQSPLSLPVTPGEPASISCRSSQSLAKSYGNTYLS | |
| | WYLQKPGQSPQLLIYGISNRFSGVPDRFSGSGSGTDFTL | |
| | KISRVEAEDVGVYYCLQGTHQPYTFGQGTKVEIK (SEQ | |
| | ID NO: 56) | |
| Human IL10 | SPGQGTQSENSCTHFPGNLPNMLRDLRDAFSRVIKTFFQ | 57 |
| | MKDQLDNLLLKESLLEDFKGYLGCQALSEMIQFYLEE | |
| | VMPQAENQDPDIKAHVNSLGENLKTLRLRLRRCHRFL | |
| | PCENKSKAVEQVKNAFNKLQEKGIYKAMSEFDIFINYI | |
| | EAYMTMKIRN | |
| Clotting Factor | ANSFLEEMKKGHLERECMEETCSYEEAREVFEDSDKT | 58 |
| X | NEFWNKYKDGDQCETSPCQNQGKCKDGLGEYTCTCL | |
| | EGFEGKNCELFTRKLCSLDNGDCDQFCHEEQNSVVCS | |
| | CARGYTLADNGKACIPTGPYPCGKQTLERRKRSVAQA | |
| | TSSSGEAPDSITWKPYDAADLDPTENPFDLLDFNQTQP | |
| | ERGDNNLTRIVGGQECKDGECPWQALLINEENEGFCG | |
| | GTILSEFYILTAAHCLYQAKRFKVRVGDRNTEQEEGGE | |
| | AVHEVEVVIKEINRFTKETYDFDIAVLRLKTPITFRMNV | |
| | APACLPERDWAESTLMTQKTGIVSGFGRTHEKGRQST | |
| | RLKMLEVPYVDRNSCKLSSSFHTQNMFCAGYDTKQED | |
| | ACQGDSGGPHVTRFKDTYFVTGIVSWGEGCARKGKY | |
| | GIYTKVTAFLKWIDRSMKTRGLPKAKSHAPEVITSSPL | |
| | K | |

TABLE 12
Exemplary Dengue virus construct sequences

| Description | Sequence | SEQ ID NO: |
|---|---|---|
| 5′UTR - | AGTTGTTAGTCTACGTGGACCGACAAGAACAGTTTCGA | 1 |
| eGFP -3′ | ATCGGAAGCTTGCTTAACGTAGTTCTGACAGTTTTTTAT | |
| UTR | TTAGAGAGCAGATCTCTGATGGTGAGCAAGGGCGAGG | |
| (Positive | AGCTCTTCACCGGGGTGGTGCCCATCCTGGTCGAGCTG | |
| strand) | GACGGCGACGTAAACGGCCACAAGTTCAGCGTGTCCG | |
| | GCGAGGGCGAGGGCGATGCCACCTACGGCAAGCTGAC | |
| | CCTGAAGTTCATCTGCACCACCGGCAAGCTGCCCGTGC | |
| | CCTGGCCCACCCTCGTGACCACCCTGACCTACGGCGTG | |
| | CAGTGCTTCAGCCGCTACCCCGACCACATGAAGCAGCA | |
| | CGACTTCTTCAAGTCCGCCATGCCCGAAGGCTACGTCC | |
| | AGGAGCGCACCATCTTCTTCAAGGACGACGGCAACTA | |
| | CAAGACCCGCGCCGAGGTGAAGTTCGAGGGCGACACC | |
| | CTGGTGAACCGCATCGAGCTGAAGGGCATCGACTTCA | |
| | AGGAGGACGGCAACATCCTGGGGCACAAGCTGGAGTA | |
| | CAACTACAACAGCCACAACGTCTATATCATGGCCGACA | |
| | AGCAGAAGAACGGCATCAAGGTGAACTTCAAGATCCG | |
| | CCACAACATCGAGGACGGCAGCGTGCAGGTCGCCGAC | |
| | CACTACCAGCAGAACACCCCCATCGGCGACGGCCCCG | |
| | TGCTGCTGCCCGACAACCACTACCTGAGCACCCAGTCC | |
| | GCCCTGAGCAAAGACCCCAACGAGAAGCGCGATCACA | |
| | TGGTCCTGCTGGAGTTCGTGACCGCCGCCGGGATCACT | |
| | CTCGGCATGGACGAGCTGTACAAGTAAGCGGCCGCTT | |
| | AATTAAGCTGCCTTCTGCGGGGCTTGCCTTCTGGCCAT | |
| | GCCCTTCTTCTCTCCCTTGCACCTGTACCTCTTGGTCTT | |
| | TGAATAAAGCCTGAGTAGGAAGTCTAGTCACAAAACA | |
| | AAGGAAAATAAGAAATCANACAAGGCAAGAAGTCAG | |
| | GCCGGATTAAGCCATAGTACGGTAAGAGCTATGCTGCC | |
| | TGTGAGCCCCGTCTAAGGACGTAAAATGAAGTCAGGC | |
| | CGGAAGCCACGGTTTGAGCAAACCGTGCTGCCTGTAGC | |
| | TCCATCGTGGGGATGTAAAAACCCGGGAGGCTGCAAC | |
| | CCATGGAAGCTGTACGCATGGGGTAGCAGACTAGTGG | |
| | TTAGAGGAGACCCCTCCCAAAACACAACGCAGCAGCG | |
| | GGGCCCAACACCAGGGGAAGCTGTACCCTGGTGGTAA | |
| | GGACTAGAGGTTAGAGGAGACCCCCCGCACAACAATA | |
| | AACAGCATATTGACGATGGGAGAGACCAGAGATCCTG | |
| | CTGTCTCTACAGCATCATTCCAGGCACAGAACGCCAGA | |
| | AAATGGAATGGTGCTGTTGAATCAACAGGTTCT | |
| 5′UTR - | TCAACAATCAGATGCACCTGGCTGTTCTTGTCAAAGCT | 2 |
| eGFP -3′ | TAGCCTTCGAACGAATTGCATCAAGACTGTCAAAAAAT | |
| UTR | AAATCTCTCGTCTAGAGACTACCACTCGTTCCCGCTCC | |
| (Negative | TCGACAAGTGGCCCCACCACGGGTAGGACCAGCTCGA | |
| strand) | CCTGCCGCTGCATTTGCCGGTGTTCAAGTCGCACAGGC | |
| | CGCTCCCGCTCCCGCTACGGTGGATGCCGTTCGACTGG | |
| | GACTTCAAGTAGACGTGGTGGCCGTTCGACQGGCACG | |
| | GGACCGGGTGGGAGCACTGGTGGGACTGGATGCCGCA | |
| | CGTCACGAAGTCGGCGATGGGGCTGGTGTACTTCGTCG | |
| | TGCTGAAGAAGTTCAGGCGGTACGGGCTTCCGATGCA | |
| | GGTCCTCGCGTGGTAGAAGAAGTTCCTGCTGCCGTTGA | |
| | TGTTCTGGGCGCGGCTCCACTTCAAGCTCCCGCTGTGG | |
| | GACCACTTGGCGTAGCTCGACTTCCCGTAGCTGAAGTT | |
| | CCTCCTGCCGTTGTAGGACCCCGTGTTCGACCTCATGTT | |
| | GATGTTGTCGGTGTTGCAGATATAGTACCGGCTGTTCG | |
| | TCTTCTTGCCGTAGTTCCACTTGAAGTTCTAGGCGGTGT | |
| | TGTAGCTCCTGCCGTCGCACGTCGAGCGGCTGGTGATG | |
| | GTCGTCTTGTGGGGGTAGCCGCTGCCGGGGCACGACG | |
| | ACGGGCTGTTGGTGATGGACTCGTGGGTCAGGCGGGA | |
| | CTCGTTTCTGGGGTTGCTTTTCGCGCTAGTGTACCAGG | |
| | ACGACCTCAAGCACTGGCGGCGGCCCTAGTGAGAGCC | |
| | GTACCTGCTCGACATGTTCATTCGCCGGCGAATTAATT | |
| | CGACGGAAGACGCCCCGAACGGAAGACCGGTACGGGA | |
| | AGAAGAGAGGGAACGTGGACATGGAGAACCAGAAAC | |
| | TTATTTCGGACTCATCCTTCAGATCAGTGTTTTGTTTCC | |
| | TTTTATTCTTTAGTTTGTTCCGTTCTTCAGTCCGGCCTA | |
| | ATTCGGTATCATGCCATTCTCGATACGACGGACACTCG | |
| | GGGCAGATTCCTGCATTTTACTTCAGTCCGGCCTTCGG | |
| | TGCCAAACTCGTTTGGCACGACGGACATCGAGGTAGC | |
| | ACCCCTACATTTTTGGGCCCTCCGACGTTGGGTACCTTC | |
| | GACATGCGTACCCCATCGTCTGATCACCAATCTTCCTCT | |
| | GGGGAGGGTTTTGTGTTGCGTCGTCGCCCCGGGTTGTG | |
| | GTCCCCTTCGACATGGGACCACCATTCCTGATCTCCAA | |
| | TCTCCTCTGGGGGGCGTGTTGTTATTTGTCGTATAACTG | |
| | CTACCCTCTCTGGTCTCTAGGACGACAGAGATGTCGTA | |
| | GTAAGGTCCGTGTCTTGCGGTCTTTTACCTTACCACGA | |
| | CAACTTAGTTGTCCAAGA | |
| 5′ UTR - | TCAACAATCAGATGCACCTGGCTGTTCTTGTCAAAGCT | 3 |
| eGFP | TAGCCTTCGAACGAATTGCATCAAGACTGTCAAAAAAT | |
| (Negative | AAATCTCTCGTCTAGAGACTACCACTCGTTCCCGCTCC | |
| strand) | TCGACAAGTGGCCCCACCACGGGTAGGACCAGCTCGA | |
| | CCTGCCGCTGCATTTGCCGGTGTTCAAGTCGCACAGGC | |
| | CGCTCCCGCTCCCGCTACGGTGGATGCCGTTCGACTGG | |
| | GACTTCAAGTAGACGTGGTGGCCGTTCGACGGGCACG | |
| | GGACCGGGTGGGAGCACTGGTGGGACTGGATGCCGCA | |
| | CGTCACGAAGTCGGCGATGGGGCTGGTGTACTTCGTCG | |
| | TGCTGAAGAAGTTCAGGCGGTACGGGCTTCCGATGCA | |
| | GGTCCTCGCGTGGTAGAAGAAGTTCCTGCTGCCGTTGA | |
| | TGTTCTGGGCGCGGCTCCACTTCAAGCTCCCGCTGTGG | |
| | GACCACTTGGCGTAGCTCGACTTCCCGTAGCTGAAGTT | |
| | CCTCCTGCCGTTGTAGGACCCCGTGTTCGACCTCATGTT | |
| | GATGTTGTCGGTGTTGCAGATATAGTACCGGCTGTTCG | |
| | TCTTCTTGCCGTAGTTCCACTTGAAGTTCTAGGCGGTGT | |
| | TGTAGCTCCTGCCGTCGCACGTCGAGCGGCTGGTGATG | |
| | GTCGTCTTGTGGGGGTAGCCGCTGCCGGGGCACGACG | |
| | ACGGGCTGTTGGTGATGGACTCGTGGGTCAGGCGGGA | |
| | CTCGTTTCTGGGGTTGCTCTTCGCGCTAGTGTACCAGG | |
| | ACGACCTCAAGCACTGGCGGCGGCCCTAGTGAGAGCC | |
| | GTACCTGCTCGACATGTTCATTCGCCGGCGAATTAATT | |
| | CGACGGAAGACGCCCCGAACGGAAGACCGGTACGGGA | |
| | AGAAGAGAGGGAACGTGGACATGGAGAACCAGAAAC | |
| | TTATTTCGGACTCATCCTTCAGATC | |
| eGFP - 3′ | TACCACTCGTTCCCGCTCCTCGACAAGTGGCCCCACCA | 4 |
| UTR | CGGGTAGGACCAGCTCGACCTGCCGCTGCATTTGCCGG | |
| (Negative | TGTTCAAGTCGCACAGGCCGCTCCCGCTCCCGCTACGG | |
| strand) | TGGATGCCGTTCGACTGGGACTTCAAGTAGACGTGGTG | |
| | GCCGTTCGACGGGCACGGGACCGGGTGGGAGCACTGG | |
| | TGGGACTGGATGCCGCACGTCACGAAGTCGGCGATGG | |
| | GGCTGGTGTACTTCGTCGTGCTGAAGAAGTTCAGGCGG | |
| | TACCGGGCTTCCGATGCAGGTCCTCGCGTGGTAGAAGAA | |
| | GTTCCTGCTGCCGTTGATGTTCTGGGCGCGGCTCCACTT | |
| | CAAGCTCCCGCTGTGGGACCACTTGGCGTAGCTCGACT | |
| | TCCCGTAGCTGAAGTTCCTCCTGCCGTTGTAGGACCCC | |
| | GTGTTCGACCTCATGTTGATGTTGTCGGTGTTGCAGAT | |
| | ATAGTACCGGCTGTTCGTCTTCTTGCCGTAGTTCCACTT | |
| | GAAGTTCTAGGCGGTGTTGTAGCTCCTGCCGTCGCACG | |
| | TCGAGCGGCTGGTGATGGTCGTCTTGTGGGGGTAGCCG | |
| | CTGCCGGGGCACGACGACGGGCTGTTGGTGATGGACT | |
| | CGTGGGTCAGGCGGGACTCGTTTCTGGGGTTGCTTTTC | |
| | GCGCTAGTGTACCAGGACGACCTCAAGCACTGGCGGC | |
| | GGCCCTAGTGAGAGCCGTACCTGCTCGACATGTTCATT | |
| | CGCCGGCGAATTAATTCGACGGAAGACGCCCCGAACG | |
| | GAAGACCGGTACGGGAAGAAGAGAGGGAACGTGGAC | |
| | ATGGAGAACCAGAAACTTATTTCGGACTCATCCTTCAG | |
| | ATCAGTGTTTTGTTTCCTTTTATTCTTTAGTTTGTTCCGT | |
| | TCTTCAGTCCGGCCTAATTCGGTATCATGCCATTCTCGA | |
| | TACGACGGACACTCGGGGCAGATTCCTGCATTTTACTT | |
| | CAGTCCGGCCTTCGGTGCCAAACTCGTTTGGCACGACG | |
| | GACATCGAGGTAGCACCCCTACATTTTTGGGCCCTCCG | |
| | ACGTTGGGTACCTTCGACATGCGTACCCCATCGTCTGA | |
| | TCACCAATCTCCTCTGGGGAGGGTTTTGTGTTGCGTCG | |
| | TCGCCCCGGGTTGTGGTCCCCTTCGACATGGGACCACC | |
| | ATTCCTGATCTCCAATCTCCTCTGGGGGGCGTGTTGTTA | |
| | TTTGTCGTATAACTGCTACCCTCTCTGGTCTCTAGGACG | |
| | ACAGAGATGTCGTAGTAAGGTCCGTGTCTTGCGGTCTT | |
| | TTACCTTACCACGACAACTTAGTTGTCCAAGA | |

### EXAMPLES

### Example 1. Producing Erythroid Cells Comprising an RNA that Comprises a Flavivirus 5′ UTR Module and a Sequence Encoding an Exogenous Protein

This Example describes introducing an RNA construct into an erythroid cell in order to express an exogenous therapeutic protein encoded on the RNA construct. The RNA construct contains, minimally, a flaviviral 5′ UTR module and a coding region for the exogenous therapeutic protein. The RNA may further comprise a flaviviral 3′ UTR. An RNA encoding NS5 (an RNA dependent RNA polymerase) is also introduced into the cell, either in the same bicistronic RNA construct or as a separate monocistronic construct. In embodiments, upon introduction of the RNAs into the cells, the RNA polymerase will be produced, will recognize the flaviviral 5′ UTR, and will replicate the RNA encoding the therapeutic protein. Without wishing to be bound by theory, an RNA having a flaviviral 5′ UTR may undergo amplification linearly, while an RNA having a flaviviral 5′ UTR and flaviviral a 3′ UTR may be amplified exponentially (See FIGS. 2A and 2B). In either case, the cell will then translate the therapeutic protein.

Several RNA configurations are shown in FIGS. 1A-IQ. FIG. 1A-1E show exemplary monocistronic RNA constructs comprising a flaviviral 5′ UTR and a coding region for an exogenous therapeutic protein (“mRNA”). FIG. 1F-1M show monocistronic RNA constructs comprising a coding region for the NS5 RNA-dependent RNA polymerase. FIG. 1N-1U show bicistronic RNA constructs comprising a flaviviral 5′ UTR, a coding region for an exogenous therapeutic protein (“mRNA”), and a coding region for the NS5 RNA-dependent RNA polymerase.

The constructs are now described in more detail. The RNA construct of FIG. 1A comprises, in the 5′ to 3′ direction, a flaviviral 5′ UTR, a positive strand exogenous mRNA sequence encoding a therapeutic protein, and a flaviviral 3′ UTR. The positive strand mRNA sequence may be directly translated into a protein by the cellular translation machinery. In embodiments, replication of an RNA construct of FIG. 1A by the RNA-dependent RNA polymerase will result in exponential amplification of the positive strand RNA construct as well as the corresponding negative strand RNA. The RNA construct of FIG. 1B, in contrast, comprises a negative strand mRNA sequence (which is not the translated strand) flanked by the reverse complement of a flaviviral 5′UTR (located 3′ relative to the negative strand mRNA sequence) and the reverse complement of a flaviviral 3′UTR (located 5′ relative to the negative strand mRNA sequence). This construct will, in embodiments, result in exponential amplification of the positive and negative strands, similar to the RNA construct of FIG. 1A. The RNA construct of FIG. 1C comprises a negative strand mRNA sequence and the reverse complement of a flaviviral 5′UTR located 3′ relative to the negative strand mRNA sequence. In embodiments, constructs in which the reverse complement of a flaviviral 5′ UTR is present and the reverse complement of a 3′ UTR is not present will undergo replication that results in linear amplification of the corresponding positive strand RNA. The RNA construct of FIG. 1D comprises the reverse complement of a flaviviral 3′ UTR located 5′ relative to the negative strand mRNA sequence. In embodiments, the RNA construct of FIG. 1D is a negative control which is not amplified due to the absence of the flavivirus 5′ UTR or its reverse complement. The RNA construct of FIG. 1E comprises, in order from 5′ to 3′ end, a flaviviral 5′ UTR, a negative strand mRNA sequence, and a flaviviral 3′ UTR. This construct will, in embodiments, result in exponential amplification of the positive and negative strands (similar to the constructs of FIGS. 1A and 1B which also have two UTRs each). The positive strand can then be translated.

An RNA construct comprising an exogenous mRNA sequence according to any of FIGS. 1A-1E is introduced into an erythroid cell in combination with a construct encoding an RNA-dependent RNA polymerase. This will allow the cell to express the polymerase which can then amplify the RNA construct of any of FIGS. 1A-1E. In this Example, the polymerase is a flaviviral NS5 RNA polymerase. RNA constructs comprising an NS5 RNA polymerase-encoding sequence or the reverse complement thereof, are shown in FIG. 1F-1M. As described in more detail below, certain of these constructs are suitable for producing therapeutic cell systems and some are more suitable as negative controls. The RNA encoding the polymerase can be flanked by a flaviviral 5′UTR and/or a flaviviral 3′ UTR. More specifically, FIGS. 1F-II show positive strands (which can be translated) encoding the RNA polymerase. The construct of FIG. 1F lacks flaviviral 5′ UTRs and, in embodiments, will be translated but not amplified, to produce a low level of RNA polymerase. The construct of FIG. 1G contains a flaviviral 5′ UTR and a flaviviral 3′ UTR and, in embodiments, will be translated and amplified exponentially, to produce a high level of RNA polymerase. The construct of FIG. 1H contains a flaviviral 5′ UTR and, in embodiments, will be translated and amplified linearly to produce non-translatable negative strands, and a low level of RNA polymerase will be produced from the positive strand. In embodiments, the construct of FIG. 1H will produce the same amount of RNA polymerase as a construct of FIG. 1F that lacks flavivirus UTRs. The construct of FIG. 1I contains a flaviviral 3′ UTR and, in embodiments, will be translated and not amplified, to produce a low level of RNA polymerase. FIGS. 1J-1M show negative strands comprising the reverse complement of the RNA polymerase-encoding sequence. The negative strands are not translated, and thus when a negative strand encoding the RNA polymerase is introduced into the cell, a small amount of RNA-dependent RNA polymerase protein or positive strand encoding the same can be introduced at the same time to perform the initial round of replication. The construct of FIG. 1J lacks flaviviral UTRs, and in embodiments, is a negative control that will not be translated or amplified. The construct of FIG. 1K comprises a flaviviral 5′ UTR and a flaviviral 3′ UTR, and in embodiments, will be exponentially amplified to form both positive and negative strands; the positive strand can then be translated to produce the polymerase protein. The construct of FIG. 1L comprises a flaviviral 5′ UTR, and in embodiments, will be linearly amplified to form positive strands; the positive strands can then be translated to produce polymerase protein. The construct of FIG. 1M lacks the flaviviral 5′ UTR, and in embodiments, is a negative control that will not be translated or amplified.

In contrast to the monocistronic RNAs described above, bicistronic RNAs can encode both the exogenous therapeutic mRNA sequence and the RNA polymerase. These are illustrated in FIGS. 1N-1U. Specifically, FIGS. 1N and 1O show the positive strands comprising exogenous mRNA and NS5 polymerase-encoding sequence (in both orientations) flanked by a flaviviral 5′ UTR and a flaviviral 3′ UTR. In embodiments, these constructs will be will be translated and amplified exponentially, to produce a high level of RNA polymerase and a high level of the exogenous therapeutic protein. FIGS. 1P and 1Q show the negative strands comprising the reverse complement of the coding region of the exogenous protein and the polymerase (in both orientations) flanked by a flaviviral 5′ UTR and a flaviviral 3′ UTR. In embodiments, these constructs will be will be translated and amplified exponentially, to produce a high level of RNA polymerase and a high level of the exogenous therapeutic protein. As discussed above, when the negative strand of the polymerase gene is used, a small amount of purified polymerase protein can be introduced to perform the initial round of replication. FIGS. 1R and 1S show the negative strands comprising the reverse complement of the coding region of the exogenous therapeutic protein and the polymerase (in both orientations) flanked by a flaviviral 5′ UTR only. In embodiments, these constructs will be will be translated and amplified linearly, to produce a low level of RNA polymerase and a low level of the exogenous therapeutic protein. The constructs of FIGS. 1T and 1U lack the flavivirus 5′ UTR or its reverse complement, and in embodiments, are negative controls that will not be translated or amplified.

The RNA constructs described above are introduced into erythroid cells by electroporation. The mRNA can be purchased from a commercial vendor (e.g., IDT-DNA, Coralville Iowa). CD34+ cells are isolated from peripheral blood by supermagnetic microbead selection by the use of Mini-MACS columns (Miltenyi Biotec; 94%+/−3% purity). Electroporation parameters can be optimized by transfecting erythroid lineage cells with mRNA transcripts and measuring transfection efficiency by quantitative RT-PCR with primers designed to specifically detect the exogenous transcripts. For certain cell preparations, discharging a 150 uF capacitor into 2.5×10⁶ cells suspended in 50 μl of Opti-MEM (Invitrogen, Carlsbad, Calif.) in a standard electroporation cuvette with a 2 mm gap is sufficient for repeated delivery in excess of 10,000 copies of modified mRNA transcripts per cell, as determined using the standard curve method, while maintaining high viability (>70%). Cell density may vary from 1×10⁶ cells/50 μl to a density of 2.5×10⁶ cells/50 μl and use from 110V to 145V to electroporate cells with similar efficiencies measured in transcript copies per cell.

Next, the electroporated erythroid cells are incubated under conditions that promote differentiation and enucleation. The cells are cultured in erythroid differentiation medium (EDM) on the basis of IMDM supplemented with stabilized glutamine, 330 μg/mL holo-human transferrin, 10 μg/mL recombinant human insulin, 2 IU/mL heparin, and 5% solvent/detergent virus-inactivated plasma. The expansion procedure comprises 3 steps. In the first step (day 0 to day 7), 10⁴/mL CD34+ cells are cultured in EDM in the presence of 1 μM hydrocortisone, 100 ng/mL SCF, 5 ng/mL IL-3, and 3 IU/mL EPO. On day 4, 1 volume of cell culture is diluted in 4 volumes of fresh medium containing SCF, IL-3, EPO, and hydrocortisone. In the second step (day 7 to day 11), the cells are resuspended at 10⁵/mL in EDM supplemented with SCF and EPO. In the third step (day 11 to day 18), the cells are cultured in EDM supplemented with EPO alone. Cell counts are adjusted to 7.5×10⁵ to 1×10⁶ and 5-10×10⁶ cells/mL on days 11 and 15, respectively. Beyond day 18, the culture medium containing EPO is renewed twice a week. The cultures are maintained at 37° C. in 5% CO2 in air.

Finally, expression of the RNA constructs is assayed. Levels of the mRNA can be measured, for example, using reverse-transcriptase PCR of samples from cells, followed by real-time PCR, e.g., as described in Example 8 of PCT Publication No. WO/2015/073587. Expression of the protein can be tested, for example, by measuring levels of fluorescence from a reporter protein (such as GFP) by flow cytometry, or by performing a Western blot on samples from cells, e.g., as described in Example 33 of PCT Publication No. WO/2015/073587.

### Example 2. Producing Erythroid Cells Comprising an RNA that Comprises a Flavivirus 5′ UTR Module and a Sequence Encoding an Exogenous Protein, and Lacks Functional Flavivirus Structural Proteins

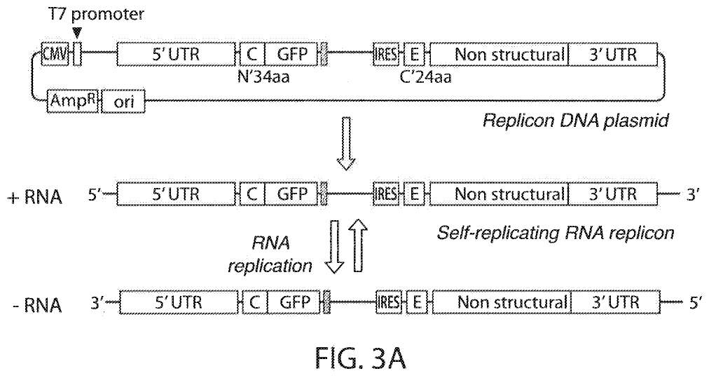

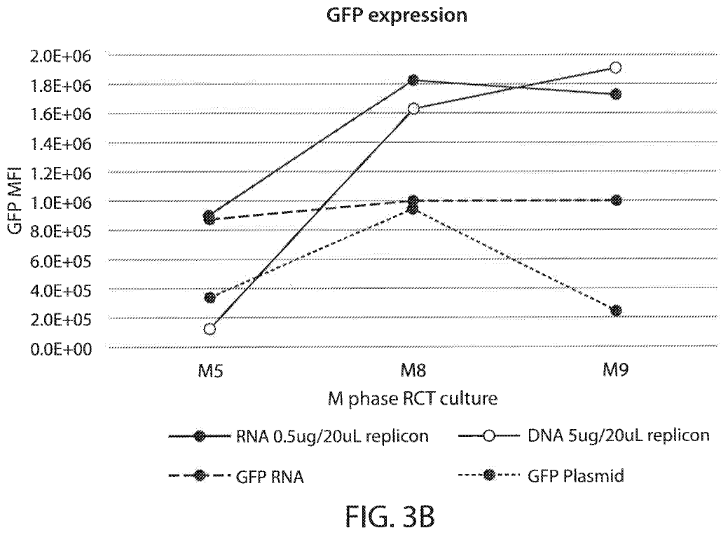

This Example describes the introduction of a Dengue virus replicon RNA or DNA into erythroid cells to express a GFP reporter protein encoded on the construct. The RNA replicon comprised, in order from 5′ to 3′, a Dengue virus 5′ UTR, a sequence encoding a nonfunctional 34 amino acid fragment of a Dengue virus core protein (C), a gene encoding GFP, an FMDV2A cleavage site, an internal ribosomal entry site (IRES), a sequence encoding a nonfunctional 24-amino acid fragment of a Dengue virus envelope (E) protein, a sequence encoding Dengue virus nonstructural proteins, and a Dengue virus 3′ UTR. This RNA replicon was produced from a circular DNA plasmid comprising the RNA replicon sequence flanked by a CMV promoter and a T7 promoter on the 5′ side and an origin of replication and an Amp^{R} gene on the 3′ side (see FIG. 3A). The circular DNA plasmid itself was also introduced into erythroid cells for expression of the GFP reporter protein.

Primary CD34+ hematopoietic stem cells were isolated from a human donor and raised in serum based media. Erythroid cells grown in culture undergo three phases—an initial stem cell expansion phase (E-phase), followed by a differentiation phase (D-phase) generating erythroid progenitor and precursor cells, and then by a maturation phase (M-phase) producing mature erythroid cells. The replicon RNA and the DNA plasmid comprising the replicon sequence were each electroporated into erythroid precursor cells that had been incubated in culture until day 4 of maturation phase (M4 cells). As positive control conditions. M4 cells were also electroporated with either GFP mRNA or a DNA expression plasmid encoding GFP. For each condition, cells were electroporated with the appropriate construct, 5 ug of the replicon RNA or the DNA plasmid were electroporated per 20 uL reaction, 4 ug of the control GFP mRNA or DNA expression plasmid encoding GFP were used per reaction.

Suitable electroporation conditions include the following. First, M4 cells are harvested from a culture dish and then centrifuged at 2000 rpm for 5 minutes to pellet. The cells are then washed with 1 mL PBS and re-centrifuged. The cell pellet is then resuspended in 20 uL of electroporation buffer for each 1×10⁶-cell reaction, and then electroporated with the appropriate construct.

Both the replicon RNA and the DNA plasmid comprising the replicon sequence were successfully introduced into M4 cells. After incubation for one day (until M5), four days (until M8), or five days (until M9), samples were taken from each set of M4 cells and mean fluorescence intensity (MFI) was measured for each sample by flow cytometry. As shown in FIG. 3B, cells electroporated with either the RNA replicon or the DNA plasmid comprising the RNA replicon sequence were capable of producing high levels of GFP protein. Indeed, these cells generated greater GFP signal than that observed for the two positive controls at days M8 and M9. In addition, GFP protein levels were as observed to increase over M5 through M9 in replicon-electroporated cells, but not in the cells with the positive control constructs.

In a separate experiment, human hematopoietic stem cells were isolated and cultured as described above. The E-, D-, and M-phase cells were then electroporated with either 1 ug or 2 ug of RNA replicon, 4 ug of positive control GFP plasmid, or 4 ug of positive control GFP mRNA. The electroporated cells were then incubated and sampled the following days in culture. Samples were stained with Annexin V and Draq5 and analyzed by flow cytometry. Cells electroporated with 1 ug or 2 ug of the RNA replicon showed strong expression of the GFP reporter throughout the sampling period. GFP expression by these cells was equal or greater than that observed for cells transfected with the positive control GFP plasmid and GFP mRNA at 24 h post transfection. Cells transfected with the positive control GFP mRNA showed a more rapid decrease in GFP expression compared to replicon transfected cells. Similar results were observed in an experiment in which murine erythroid cell precursors were transfected with each of the above-described constructs.

In a further experiment, an RNA replicon variant comprising a gene encoding GFP and an exogenous arginase, erwASNase, was tested. Transgene expression was observed at multiple timepoints.

### Example 3. Translation of Exemplary RNA Constructs by K562 Cells

In this example, several exemplary RNA constructs were designed and tested for their capacity to be translated into protein in the erythroleukemia cell line. K562. Five RNA constructs were designed, each of which included a 5′ Cap (CleanCap Cap1 (TRILINK)). The first RNA construct consisted of, from 5′ to 3′, a 5′ alpha globin UTR, a the eGFP-encoding sequence (“GFP”), and a 3′ alpha globin UTR. The second construct consisted of, from 5′ to 3′, a Dengue virus Type II-derived RdRP-responsive 5′ UTR (“5-AMP”), a eGFP-encoding sequence, and Dengue virus Type II-derived RdRP-responsive 3′ UTR. (“3-AMP”). The third construct consisted of, from 5′ to 3′, a Dengue virus Type II-derived RdRP-responsive 5′ UTR, a eGFP-encoding sequence, a Dengue virus Type II-derived RdRP-responsive 3′ UTR, and a 120 nt-poly-A tail. The fourth RNA construct consisted of, from 5′ to 3′, a Dengue virus Type II-derived RdRP-responsive 5′ UTR, an MSCV IRES (SEQ ID NO: 39), a 5′ alpha globin UTR, eGFP-encoding sequence, a 3′ alpha globin UTR, and a Dengue virus Type II-derived RdRP-responsive 3′ UTR. The fifth RNA construct consist of, from 5′ to 3′, a Dengue virus Type II-derived RdRP-responsive 5′ UTR. IRES, GFP-encoding sequence, a Dengue virus Type II-derived RdRP-responsive 3′ UTR, and a 120 nt poly-A tail. Briefly, the RNA constructs were electroporated into K562 erythroleukemia cells using a Lonza 4D nucleofector (2.5×10⁶ cells with 3 μg of RNA construct per reaction). Untreated (non-electroporated cells; “No EP”) were used as control. Cells were grown in culture media (Iscove's Modified Dulbecco's Medium+10% fetal bovine serum) for 24 hours. GFP expression was analyzed using flow cytometry.

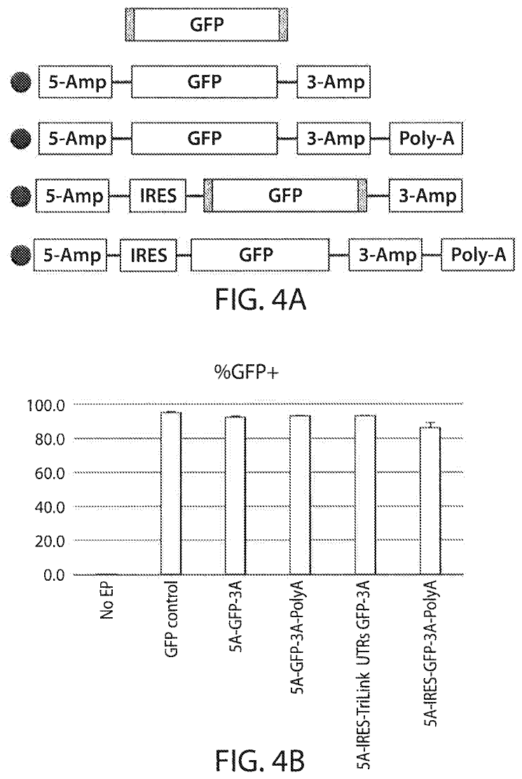

As shown in FIG. 4B, GFP signal was detected in each of the conditions electroporated with RNA constructs. These data indicate that all RNA constructs were translationally competent when introduced into the cells demonstrating that the Dengue virus Type II-derived RdRP-responsive 5′ and 3′ UTRs did not interfere with the ability of the translational machinery to produce protein.

1. An erythroid cell comprising a nucleic acid molecule comprising a flavivirus 5′ UTR and an exogenous nucleic acid sequence comprising a non-flaviviral gene.

2. An enucleated erythroid cell comprising an exogenous non-flaviviral protein or an exogenous noncoding RNA, wherein the exogenous non-flaviviral protein or the exogenous noncoding RNA was produced by a precursor of the enucleated erythroid cell from a nucleic acid molecule comprising a flavivirus 5′ UTR and an exogenous nucleic acid sequence comprising a gene encoding the exogenous non-flaviviral protein or noncoding RNA.

3. The cell of any of the preceding claims, wherein the nucleic acid molecule does not comprise a nucleic acid sequence encoding one or more functional flavivirus structural proteins.

4. The cell of claim 3, wherein the flavivirus structural protein is prM protein.

5. The cell of any of the preceding claims, wherein the nucleic acid molecule does not comprise any nucleic acid sequences encoding flavivirus structural proteins chosen from flavivirus C, and E proteins.

6. The cell of any of the preceding claims, wherein the nucleic acid molecule does not encode a flavivirus nonstructural protein selected from NS1, NS2A, NS2B, NS3, NS4A, NS4B, and NS5, or any combination thereof.

7. The cell of any of the preceding claims, wherein the nucleic acid molecule comprises a nucleic acid sequence encoding one or more flavivirus nonstructural proteins.

8. The cell of claim 7, wherein the one or more flavivirus nonstructural proteins comprises an RdRP and/or a helicase.

9. The cell of any of the preceding claims, wherein the cell further comprises a second exogenous nucleic acid molecule encoding a functional RNA-dependent RNA polymerase (RdRP).

10. The cell of claim 9, wherein the cell further comprises a third exogenous nucleic acid encoding a nuclease and/or a helicase.

11. The cell of any of the preceding claims, wherein the cell further comprises a functional RNA-dependent RNA polymerase (RdRP).

12. The cell of any of the preceding claims, further comprising an exogenous non-flaviviral protein encoded by the non-flaviviral gene.

13. The cell of any of the preceding claims, wherein the non-flaviviral gene encodes an exogenous protein or functional RNA.

14. The cell of any of the preceding claims, wherein the non-flaviviral gene encodes a therapeutic agent.

15. The cell of any of the preceding claims, wherein the non-flaviviral gene encodes an antibody molecule, enzyme, cytokine, cytokine receptor, chemokine, chemokine receptor, interleukin, receptor, ligand, hormone, growth factor, blood factor, lysosomal storage enzyme, immune stimulatory molecule, intein, DNA-binding protein, RNA-binding protein, a complement regulatory molecule, a complement cascade molecule, a clotting cascade molecule, a chelator, or a functional fragment thereof.

16. The cell of any of the preceding claims, wherein the nucleic acid further comprises an RdRP-responsive 3′ UTR.

17. The cell of claim 16, wherein the RdRP-responsive 3′ UTR comprises a 3′ flavivirus UTR.

18. A nucleic acid construct comprising a flavivirus 5′ UTR and an exogenous nucleic acid sequence comprising a non-flaviviral gene, wherein the nucleic acid construct does not comprise any sequences encoding flavivirus structural proteins.

19. The nucleic acid construct of claim 18, wherein the flavivirus structural proteins are selected from flavivirus C, prM, or E protein, or any combination thereof.

20. A nucleic acid construct comprising a flavivirus 5′ UTR and an exogenous nucleic acid sequence comprising a non-flaviviral gene, wherein the nucleic acid construct does not comprise any sequences encoding flavivirus nonstructural proteins.

21. The nucleic acid construct of claim 20, wherein the nucleic acid construct does not comprise any sequences encoding genes encoding flavivirus NS1, NS2A, NS2B, NS3, NS4A, NS4B, or NS5 proteins.

22. A nucleic acid construct comprising a flavivirus 5′ UTR and an exogenous nucleic acid sequence comprising a non-flaviviral gene, wherein the nucleic acid construct does not comprise any sequences encoding flavivirus NS1, NS2A, NS2B, NS3, NS4A, or NS4B proteins.

23. A nucleic acid construct comprising a flavivirus 5′ UTR and lacking at least a portion of a gene encoding a flavivirus nonstructural protein, optionally wherein the nucleic acid construct further comprises an exogenous nucleic acid sequence comprising a non-flaviviral gene.

24. The nucleic acid construct of claim 23, which lacks at least one gene encoding a functional flavivirus nonstructural protein.

25. The nucleic acid construct of claim 24, which lacks at least one gene encoding a flavivirus NS1, NS2A, NS2B, NS3, NS4A, NS4B, or NS5, or any combination thereof.

26. The nucleic acid construct of any of claims 23-25, which lacks at least one gene encoding a flavivirus nonstructural protein.

27. The nucleic acid construct of claim 26, which lacks at least one gene encoding a flavivirus NS1, NS2A, NS2B, NS3, NS4A, NS4B, or NS5, or any combination thereof.

28. The nucleic acid construct of any of claims 23-27, which lacks a gene encoding a functional flavivirus NS1 protein.

29. The nucleic acid construct of any of claims 23-25, which lacks a gene encoding a flavivirus NS1 protein.

30. The nucleic acid construct of any one of claims 23-29, which lacks at least a portion of a gene encoding a flavivirus structural protein.

31. The nucleic acid construct of any one of claims 23-30, wherein the nucleic acid construct comprises a fragment of a gene encoding a flaviviral structural protein.

32. The nucleic acid construct of any one of claims 23-31, which lacks at least one gene encoding a functional flavivirus structural protein.

33. The nucleic acid construct of claim 32, which lacks at least one gene encoding a functional flavivirus C, prM, or E protein, or any combination thereof.

34. The nucleic acid construct of any of claims 30-33, which lacks at least one gene encoding a flavivirus structural protein.

35. The nucleic acid construct of claim 34, which lacks at least one gene encoding a flavivirus C, prM, or E protein, or any combination thereof.

36. A nucleic acid construct comprising a flavivirus 5′ UTR sequence and an exogenous gene selected from:
a) exogenous primate gene;
b) a single-chain variable fragment (scFv);
c) a non-viral noncoding RNA;
d) a gene encoding a non-viral protein that is not a reporter; or
e) a gene encoding a fusion protein that comprises a first domain and a transmembrane domain.

37. The nucleic acid construct of claim 36, wherein the transmembrane domain comprises a transmembrane domain present endogenously in mammalian cells.

38. The nucleic acid construct of any of claims 18-37, which does not comprise an RdRP-responsive 3′ UTR.

39. The nucleic acid construct of any of claims 18-38, which does not comprise a flavivirus 3′ UTR.

40. The nucleic acid construct of any of claims 13-37, which further comprises an RdRP-responsive 3′ UTR.

41. The nucleic acid construct of claim 40, which comprises a flavivirus 3′ UTR.

42. The nucleic acid construct of any of claims 18-40, wherein the flavivirus is Dengue virus.

43. The nucleic acid construct of claim 42, wherein the flavivirus is a Dengue virus serotype selected from the group consisting of DEN-1, DEN-2, DEN-3, and DEN-4.

44. The nucleic acid construct of any of claims 18-43, which is an RNA construct that comprises a 5′ cap, a poly-A tail, or both of a 5′ cap and a poly-A tail.

45. The nucleic acid construct of any of claims 18-44, which is an RNA construct that comprises a poly-A tail and does not comprise a flaviviral 3′ UTR.

46. The nucleic acid construct of any of claims 18-45, which is an RNA construct that comprises a flaviviral 3′ UTR and does not comprise a poly-A tail.

47. A cell comprising the nucleic acid construct of any of claims 18-46.

48. A composition comprising:
(i) a first nucleic acid molecule comprising a flavivirus 5′ UTR and an exogenous nucleic acid sequence; wherein the first nucleic acid molecule does not comprise a sequence encoding a functional RNA-dependent RNA polymerase (RdRP), and
(ii) a second nucleic acid molecule comprising a sequence encoding a functional RdRP;
wherein the first nucleic acid molecule and the second nucleic acid molecule are each non-naturally occurring nucleic acid molecules.

49. The composition of claim 48, wherein the RdRP encoded by the second nucleic acid molecule is a flavivirus RdRP.

50. The composition of claim 49, wherein the RdRP encoded by the second nucleic acid molecule is a Dengue virus RdRP.

51. The composition of any one of claims 48-50, wherein the flavivirus 5′ UTR is a Dengue virus 5′ UTR.

52. The composition of claim 51, wherein the flavivirus 5′ UTR is a 5′UTR from a Dengue virus serotype chosen from DEN-1, DEN-2, DEN-3, or DEN-4.

53. The composition of any one of claims 48-52, wherein the first nucleic acid molecule does not comprise any sequences encoding one or more functional flavivirus structural proteins.

54. The composition of claim 53, wherein the first nucleic acid molecule does not comprise any sequences encoding one or more of flavivirus C, prM, and E proteins.

55. The composition of any one of claims 48-54, wherein the first nucleic acid molecule does not comprise any sequences encoding one or more functional flavivirus nonstructural genes.

56. The composition of claim 55, wherein the first nucleic acid molecule does not comprise any sequences encoding flavivirus NS1, NS2A, NS2B, NS3, NS4A, NS4B, and NS5 proteins.

57. The composition of claim 55, wherein the first nucleic acid molecule does not comprise any sequences encoding a functional NS1 protein.

58. The composition of claim 56, wherein the second nucleic acid molecule does not comprise any sequences encoding a functional NS1 protein.

59. The composition of any one of claims 48-58, wherein the second nucleic acid molecule does not comprise any sequences encoding one or more functional flavivirus structural proteins.

60. The composition of claim 59, wherein the second nucleic acid molecule does not comprise any sequences encoding on or more of flavivirus C, prM, and E proteins.

61. The composition of any one of claims 48-60, wherein the exogenous sequence comprises a bacterial, archaean, or eukaryotic sequence.

62. The composition of claim 61, wherein the exogenous nucleic acid sequence comprises a mammalian, plant, fungal, or insect sequence.

63. The composition of claim 62, wherein the exogenous nucleic acid sequence comprises a human sequence.

64. The composition of any one of claims 48-63, wherein the exogenous nucleic acid sequence encodes a polypeptide.

65. The composition of claim 64, wherein the exogenous nucleic acid sequence encodes an antibody molecule, or an antigen-binding fragment thereof.

66. The composition of claim 65, wherein the exogenous nucleic acid sequence encodes an scFv, or a T cell receptor, or an antigen-binding fragment thereof.

67. The composition of claim 65, wherein the exogenous nucleic acid sequence encodes a fusion protein comprising a first domain and a transmembrane domain.

68. The composition of claim 67, wherein the transmembrane domain comprises a Type I, Type II, or Type III transmembrane domain.

69. The composition of claim 67 or 68, wherein the fusion protein comprises an antibody molecule, or an antigen-binding fragment thereof.

70. The composition of claim 69, wherein the antibody molecule, or the antigen-binding fragment thereof, is an scFv.

71. The composition of any of claims 67-70, wherein the fusion protein comprises a heavy chain, a light chain, or a heavy chain and a light chain.

72. The composition of any of claims 67-71, wherein the transmembrane domain comprises a transmembrane domain present endogenously in mammalian cells.

73. The composition of claim 72, wherein the transmembrane domain comprises a transmembrane domain present endogenously in human cells, erythroid cells, or erythrocytes.

74. The composition of any one of claims 48-73, wherein the exogenous nucleic acid sequence encodes a noncoding RNA.

75. The composition of claim 74, wherein the exogenous nucleic acid sequence encodes a microRNA, ribozyme, aptamer, siRNA, piRNA, snoRNA, snRNA, long ncRNA, or tRNA.

76. The composition of any one of claims 48-75, wherein the first nucleic acid molecule does not comprise an RdRP-responsive 3′ UTR, and/or wherein the second nucleic acid molecule does not comprise an RdRP-responsive 3′ UTR.

77. The composition of any one of claims 48-76, wherein the first nucleic acid molecule further comprises an RdRP-responsive 3′ UTR, and/or wherein the second nucleic acid molecule further comprises an RdRP-responsive 3′ UTR.

78. A cell comprising the composition of any of claims 48-77.

79. The cell of claim 78, further comprising an RNA molecule comprising an RdRP-responsive 5′ UTR and/or an RdRP-responsive 3′ UTR, wherein the RNA molecule does not comprise an exogenous nucleic acid sequence encoding an exogenous protein.

80. A method of making a cell comprising an exogenous protein, the method comprising:
(i) introducing the nucleic acid construct of any one of claims 18-46 or a composition of any of claims 48-77 into a cell, and
(ii) culturing the cell under conditions whereby the exogenous nucleic acid sequence is expressed to produce an exogenous therapeutic agent;
thereby making a cell comprising a therapeutic agent.

81. A method of making a cell comprising an exogenous therapeutic agent, the method comprising:
(i) introducing the nucleic acid construct of any one of claims 18-46 or a composition of any of claims 48-77 into a cell, and
(ii) culturing the cell under conditions whereby the non-viral sequence is expressed to produce an exogenous therapeutic agent;
thereby making a cell comprising a therapeutic agent.

82. The method of claim 80 or 81, wherein the cell is a mammalian cell, a plant cell, bacterial cell, fungal cell, or insect cell.

83. A method of making an erythroid cell comprising an exogenous therapeutic agent, the method comprising:
(i) introducing the nucleic acid construct of any one of claims 18-46 or a composition of any of claims 48-77 into a nucleated erythroid cell precursor, and
(ii) culturing the nucleated erythroid cell precursor under conditions whereby the exogenous nucleic acid sequence is expressed to produce an exogenous therapeutic agent, optionally wherein the culturing results in enucleation of the nucleated erythroid cell precursor,
thereby making an erythroid cell comprising a therapeutic agent.

84. A method of making an erythroid cell comprising an exogenous therapeutic agent, the method comprising:
(i) introducing the nucleic acid construct of any one of claims 18-46 or a composition of any of claims 48-77 into a nucleated erythroid cell precursor, and
(ii) culturing the nucleated erythroid cell precursor under conditions whereby the non-viral sequence is expressed to produce an exogenous therapeutic agent, optionally wherein the culturing results in enucleation of the nucleated erythroid cell precursor;
thereby making an erythroid cell comprising a therapeutic agent.

85. The method of claim 83 or 84, wherein introducing the nucleic acid construct or composition into the cell comprises using electroporation or transfection.

86. The method of claim 85, wherein the transfection comprises LNP-mediated transfection.

87. A method of inducing delayed expression of an exogenous therapeutic agent in a cell, the method comprising:
(i) introducing into a cell a negative strand construct comprising an RdRP-dependent 5′ UTR operably linked to a nucleic acid sequence encoding the exogenous therapeutic agent,
(ii) introducing a compatible RdRP into the cell; and
(iii) culturing the cell under conditions whereby the exogenous therapeutic agent is expressed under conditions wherein a positive strand construct is produced from the negative strand construct, and wherein the positive strand construct is translated to produce the exogenous therapeutic agent;
thereby inducing delayed expression of the exogenous therapeutic agent.

88. A method of administering an enucleated erythroid cell to a subject, comprising administering to the subject an enucleated erythroid cell made according to the method of any one of claims 83-87, thereby administering the enucleated erythroid cell to the subject.

89. A method of delivering an exogenous protein to a subject, comprising administering to the subject an enucleated erythroid cell made according to the method of any one of claims 83-87, which comprises the exogenous protein, thereby delivering the exogenous protein to the subject.

90. A nucleic acid construct comprising an RdRP-responsive 5′ UTR and an exogenous nucleic acid sequence comprising an exogenous gene, wherein the nucleic acid construct does not comprise any sequences encoding viral structural proteins.

91. An erythroid cell comprising the nucleic acid construct of claim 90.

92. A nucleic acid construct according to any of FIGS. 1A-1U, or any combination thereof.


SEQUENCE LISTING (60 sequences)

SEQ ID NO: 1 - DNA, 1381 bp, Artificial Sequence
  source: /note=“Description of Artificial Sequence Synthetic polynucleotide”
agttgttagtctacgtggaccgacaagaacagtttcgaatcggaagcttgcttaacgtagttctgacagttttttatttagagagcagatctctgatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagcggccgcttaattaagctgccttctgcggggcttgccttctggccatgcccttcttctctcccttgcacctgtacctcttggtctttgaataaagcctgagtaggaagtctagtcacaaaacaaaggaaaataagaaatcaaacaaggcaagaagtcaggccggattaagccatagtacggtaagagctatgctgcctgtgagccccgtctaaggacgtaaaatgaagtcaggccggaagccacggtttgagcaaaccgtgctgcctgtagctccatcgtggggatgtaaaaacccgggaggctgcaacccatggaagctgtacgcatggggtagcagactagtggttagaggagacccctcccaaaacacaacgcagcagcggggcccaacaccaggggaagctgtaccctggtggtaaggactagaggttagaggagaccccccgcacaacaataaacagcatattgacgatgggagagaccagagatcctgctgtctctacagcatcattccaggcacagaacgccagaaaatggaatggtgctgttgaatcaacaggttct

SEQ ID NO: 2 - DNA, 1381 bp, Artificial Sequence
  source: /note=“Description of Artificial Sequence Synthetic polynucleotide”
tcaacaatcagatgcacctggctgttcttgtcaaagcttagccttcgaacgaattgcatcaagactgtcaaaaaataaatctctcgtctagagactaccactcgttcccgctcctcgacaagtggccccaccacgggtaggaccagctcgacctgccgctgcatttgccggtgttcaagtcgcacaggccgctcccgctcccgctacggtggatgccgttcgactgggacttcaagtagacgtggtggccgttcgacgggcacgggaccgggtgggagcactggtgggactggatgccgcacgtcacgaagtcggcgatggggctggtgtacttcgtcgtgctgaagaagttcaggcggtacgggcttccgatgcaggtcctcgcgtggtagaagaagttcctgctgccgttgatgttctgggcgcggctccacttcaagctcccgctgtgggaccacttggcgtagctcgacttcccgtagctgaagttcctcctgccgttgtaggaccccgtgttcgacctcatgttgatgttgtcggtgttgcagatatagtaccggctgttcgtcttcttgccgtagttccacttgaagttctaggcggtgttgtagctcctgccgtcgcacgtcgagcggctggtgatggtcgtcttgtgggggtagccgctgccggggcacgacgacgggctgttggtgatggactcgtgggtcaggcgggactcgtttctggggttgcttttcgcgctagtgtaccaggacgacctcaagcactggcggcggccctagtgagagccgtacctgctcgacatgttcattcgccggcgaattaattcgacggaagacgccccgaacggaagaccggtacgggaagaagagagggaacgtggacatggagaaccagaaacttatttcggactcatccttcagatcagtgttttgtttccttttattctttagtttgttccgttcttcagtccggcctaattcggtatcatgccattctcgatacgacggacactcggggcagattcctgcattttacttcagtccggccttcggtgccaaactcgtttggcacgacggacatcgaggtagcacccctacatttttgggccctccgacgttgggtaccttcgacatgcgtaccccatcgtctgatcaccaatctcctctggggagggttttgtgttgcgtcgtcgccccgggttgtggtccccttcgacatgggaccaccattcctgatctccaatctcctctggggggcgtgttgttatttgtcgtataactgctaccctctctggtctctaggacgacagagatgtcgtagtaaggtccgtgtcttgcggtcttttaccttaccacgacaacttagttgtccaaga

SEQ ID NO: 3 - DNA, 929 bp, Artificial Sequence
  source: /note=“Description of Artificial Sequence Synthetic polynucleotide”
tcaacaatcagatgcacctggctgttcttgtcaaagcttagccttcgaacgaattgcatcaagactgtcaaaaaataaatctctcgtctagagactaccactcgttcccgctcctcgacaagtggccccaccacgggtaggaccagctcgacctgccgctgcatttgccggtgttcaagtcgcacaggccgctcccgctcccgctacggtggatgccgttcgactgggacttcaagtagacgtggtggccgttcgacgggcacgggaccgggtgggagcactggtgggactggatgccgcacgtcacgaagtcggcgatggggctggtgtacttcgtcgtgctgaagaagttcaggcggtacgggcttccgatgcaggtcctcgcgtggtagaagaagttcctgctgccgttgatgttctgggcgcggctccacttcaagctcccgctgtgggaccacttggcgtagctcgacttcccgtagctgaagttcctcctgccgttgtaggaccccgtgttcgacctcatgttgatgttgtcggtgttgcagatatagtaccggctgttcgtcttcttgccgtagttccacttgaagttctaggcggtgttgtagctcctgccgtcgcacgtcgagcggctggtgatggtcgtcttgtgggggtagccgctgccggggcacgacgacgggctgttggtgatggactcgtgggtcaggcgggactcgtttctggggttgctcttcgcgctagtgtaccaggacgacctcaagcactggcggcggccctagtgagagccgtacctgctcgacatgttcattcgccggcgaattaattcgacggaagacgccccgaacggaagaccggtacgggaagaagagagggaacgtggacatggagaaccagaaacttatttcggactcatccttcagatc

SEQ ID NO: 4 - DNA, 1286 bp, Artificial Sequence
  source: /note=“Description of Artificial Sequence Synthetic polynucleotide”
taccactcgttcccgctcctcgacaagtggccccaccacgggtaggaccagctcgacctgccgctgcatttgccggtgttcaagtcgcacaggccgctcccgctcccgctacggtggatgccgttcgactgggacttcaagtagacgtggtggccgttcgacgggcacgggaccgggtgggagcactggtgggactggatgccgcacgtcacgaagtcggcgatggggctggtgtacttcgtcgtgctgaagaagttcaggcggtacgggcttccgatgcaggtcctcgcgtggtagaagaagttcctgctgccgttgatgttctgggcgcggctccacttcaagctcccgctgtgggaccacttggcgtagctcgacttcccgtagctgaagttcctcctgccgttgtaggaccccgtgttcgacctcatgttgatgttgtcggtgttgcagatatagtaccggctgttcgtcttcttgccgtagttccacttgaagttctaggcggtgttgtagctcctgccgtcgcacgtcgagcggctggtgatggtcgtcttgtgggggtagccgctgccggggcacgacgacgggctgttggtgatggactcgtgggtcaggcgggactcgtttctggggttgcttttcgcgctagtgtaccaggacgacctcaagcactggcggcggccctagtgagagccgtacctgctcgacatgttcattcgccggcgaattaattcgacggaagacgccccgaacggaagaccggtacgggaagaagagagggaacgtggacatggagaaccagaaacttatttcggactcatccttcagatcagtgttttgtttccttttattctttagtttgttccgttcttcagtccggcctaattcggtatcatgccattctcgatacgacggacactcggggcagattcctgcattttacttcagtccggccttcggtgccaaactcgtttggcacgacggacatcgaggtagcacccctacatttttgggccctccgacgttgggtaccttcgacatgcgtaccccatcgtctgatcaccaatctcctctggggagggttttgtgttgcgtcgtcgccccgggttgtggtccccttcgacatgggaccaccattcctgatctccaatctcctctggggggcgtgttgttatttgtcgtataactgctaccctctctggtctctaggacgacagagatgtcgtagtaaggtccgtgtcttgcggtcttttaccttaccacgacaacttagttgtccaaga

SEQ ID NO: 5 - DNA, 95 bp, Dengue virus
agttgttagtctacgtggaccgacaagaacagtttcgaatcggaagcttgcttaacgtagttctgacagttttttatttagagagcagatctctg

SEQ ID NO: 6 - DNA, 452 bp, Dengue virus
tcacaaaacaaaggaaaataagaaatcaaacaaggcaagaagtcaggccggattaagccatagtacggtaagagctatgctgcctgtgagccccgtctaaggacgtaaaatgaagtcaggccggaagccacggtttgagcaaaccgtgctgcctgtagctccatcgtggggatgtaaaaacccgggaggctgcaacccatggaagctgtacgcatggggtagcagactagtggttagaggagacccctcccaaaacacaacgcagcagcggggcccaacaccaggggaagctgtaccctggtggtaaggactagaggttagaggagaccccccgcacaacaataaacagcatattgacgatgggagagaccagagatcctgctgtctctacagcatcattccaggcacagaacgccagaaaatggaatggtgctgttgaatcaacaggttct

SEQ ID NO: 7 - DNA, 95 bp, Dengue virus
tcaacaatcagatgcacctggctgttcttgtcaaagcttagccttcgaacgaattgcatcaagactgtcaaaaaataaatctctcgtctagagac

SEQ ID NO: 8 - DNA, 452 bp, Dengue virus
agtgttttgtttccttttattctttagtttgttccgttcttcagtccggcctaattcggtatcatgccattctcgatacgacggacactcggggcagattcctgcattttacttcagtccggccttcggtgccaaactcgtttggcacgacggacatcgaggtagcacccctacatttttgggccctccgacgttgggtaccttcgacatgcgtaccccatcgtctgatcaccaatctcctctggggagggttttgtgttgcgtcgtcgccccgggttgtggtccccttcgacatgggaccaccattcctgatctccaatctcctctggggggcgtgttgttatttgtcgtataactgctaccctctctggtctctaggacgacagagatgtcgtagtaaggtccgtgtcttgcggtcttttaccttaccacgacaacttagttgtccaaga

SEQ ID NO: 9 - protein, 900 aa, Dengue virus 2
GTGNIGETLGEKWKSRLNALGKSEFQIYKKSGIQEVDRTLAKEGIKRGETDHHAVSRGSAKLRWFVERNMVTPEGKVVDLGCGRGGWSYYCGGLKNVREVKGLTKGGPGHEEPIPMSTYGWNLVRLQSGVDVFFIPPEKCDTLLCDIGESSPNPTVEAGRTLRVLNLVENWLNNNTQFCIKVLNPYMPSVIEKMEALQRKYGGALVRNPLSRNSTHEMYWVSNASGNIVSSVNMISRMLINRFTMRYKKATYEPDVDLGSGTRNIGIESEIPNLDIIGKRIEKIKQEHETSWHYDQDHPYKTWAYHGSYETKQTGSASSMVNGVVRLLTKPWDVVPMVTQMAMTDTTPFGQQRVFKEKVDTRTQEPKEGTKKLMKITAEWLWKELGKKKTPRMCTREEFTRKVRSNAALGAIFTDENKWKSAREAVEDSRFWELVDKERNLHLEGKCETCVYNMMGKREKKLGEFGKAKGSRAIWYMWLGARFLEFEALGFLNEDHWFSRENSLSGVEGEGLHKLGYILRDVSKKEGGAMYADDTAGWDTRITLEDLKNEEMVTNHMEGEHKKLAEAIFKLTYQNKVVRVQRPTPRGTVMDIISRRDQRGSGQVGTYGLNTFTNMEAQLIRQMEGEGVFKSIQHLTITEEIAVQNWLARVGRERLSRMAISGDDCVVKPLDDRFASALTALNDMGKIRKDIQQWEPSRGWNDWTQVPFCSHHFHELIMKDGRVLVVPCRNQDELIGRARISQGAGWSLRETACLGKSYAQMWSLMYFHRRDLRLAANAICSAVPSHWVPTSRTTWSIHAKHEWMTTEDMLTVWNRVWIQENPWMEDKTPVESWEEIPYLGKREDQWCGSLIGLTSRATWAKNIQAAINQVRSLIGNEEYTDYMPSMKRFRREEEEAGVLW

SEQ ID NO: 10 - DNA, 122 bp, Dengue virus 2
acaaagacagattctttgagggagctaagcttaacgtagttctaacagttttttaattagagagcagatctctgatgaacaaccaacggaaaaaggcgagaaatacgcctttcaatatgctg

SEQ ID NO: 11 - DNA, 111 bp, Dengue virus 2
acaaagacagattctttgagggagctaagcttaacgtagttctaacagttttttaattagagagcagatctctgatgaacaaccaacggaaaaaggcgagaaatacgcctt

SEQ ID NO: 12 - DNA, 90 bp, Dengue virus 2
cagcatattgacgctgggaaagaccagagatcctgctgtctcctcagcatcattccaggcacagaacgccagaaaatggaatggtgttgt

SEQ ID NO: 13 - DNA, 79 bp, Dengue virus 2
cgctgggaaagaccagagatcctgctgtctcctcagcatcattccaggcacagaacgccagaaaatggaatggtgttgt

SEQ ID NO: 14 - DNA, 160 bp, Dengue virus 2
acaaagacagattctttgagggagctaagcttaacgtagttctaacagttttttaattagagagcagatctctgatgaacaaccaacggaaaaaggcgagaaatacgcctttcaatatgctgaaacgcgagagaaaccgcgtgtcgactgtgcaacagtt

SEQ ID NO: 15 - DNA, 436 bp, Dengue virus 2
gaaggcagaattaacatgagataagattagaagtcaggtcggattaagccatagtacgggaaaaactatgctacctgtgagccccgtccaaggacgttaaaagaagtcaggccactacaagtgccataacttgagcaaactatgcagcctgtagctccacctgggaaggtgtaaaaaatctgggaggccacaaaccatggaagctgtacgcatggcgtagtggactagcggttagaggagacccctcccctgcaaatcgcagcaacaatgggggcccaaggtgagatgaagctgtagtctcactggaaggactagaggttagaggagacccccccgaaacaaaaaacagcatattgacgctgggaaagaccagagatcctgctgtctcctcagcatcattccaggcacagaacgccagaaaatggaatggtgttgt

SEQ ID NO: 16 - protein, 618 aa, Dengue virus 2
AGVLWDVPSPPPVGKAELEDGAYRIKQKGILGYSQIGAGVYKEGTFHTMWHVTRGAVLMHKGKRIEPSWADVRKDLISYGGGWKLEGEWKEGEEVQVLALEPGKNPRAVQTKPGLFKTDTGTIGAVSLDFSPGTSGSPIVDKKGKVVGLYGNGVVTRSGTYVSAIAQTEKSIEDNPEIEDDIFRKRRLTIMDLHPGAGKTKRYLPAIVREAIKRGLRTLILAPTRVVAAEMEEALRGLPIRYQTPAIRAEHTGREIVDLMCHATFTMRLLSPVRVPNYNLIIMDEAHFTDPASIAARGYISTRVEMGEAAGIFMTATPPGSRDPFPQSNAPIMDEEREIPERSWNSGHEWVTDFKGKTVWFVPSIKAGNDIAACLRKNGKKVIQLSRKTFDSEYIKTRTNDWDFVVTTDISEMGANFKAERVIDPRRCMKPVILTDGEERVILAGPMPVTHSSAAQRRGRIGRNPKNENDQYIYMGEPLENDEDCAHWKEAKMLLDNINTPEGIIPSMFEPEREKVDAIDGEYRLRGEARKTFVDLMRRGDLPVWLAYRVAAEGINYADRRWCFDGVKNNQILEENVEVEIWTKEGERKKLKPRWLDARIYSDPLALKEFKEFAAGRK

SEQ ID NO: 17 - protein, 149 aa, West Nile virus
TTGVYRIMTRGLLGSYQAGAGVMVEGVFHTLWHTTKGAALMSGEGRLDPYWGSVKEDRLCYGGPWKLQHKWNGHDEVQMIVVEPGKNVKNVQTKPGVFKTPEGEIGAVTLDYPTGTSGSPIVDKNGDVIGLYGNGVIMPNGSYISAIVQ

SEQ ID NO: 18 - protein, 651 aa, West Nile virus
EEDVNLGSGTRAVGKPLLNSDTSKIKNRIERLRREYSSTWHHDENHPYRTWNYHGSYDVRPTGSASSLVNGVVRLLSKPWDTITNVTTMAMTDTTPFGQQRVFKEKVDTKAPEPPEGVKYVLNETTNWLWAFLAREKRPRMCSREEFIRKVNSNAALGAMFEEQNQWRSAREAVEDPKFWEMVDEEREAHLRGECHTCIYNMMGKREKKPGEFGKAKGSRAIWFMWLGARFLEFEALGFLNEDHWLGRKNSGGGVEGLGLQKLGYILREVGTRPGGKIYADDTAGWDTRITRADLENEAKVLELLDGEHRRLARAIIELTYRHKVVKVMRPAADGRTVMDVISREDQRGSGQVVTYALNTFTNLAVQLVRMMEGEGVVGPDDVEKLTKGKGPKVRTWLFENGEERLSRMAVSGDDCVVKPLDDRFATSLHFLNAMSKVRKDIQEWKPSTGWYDWQQVPFCSNHFTELIMKDGRTLVVPCRGQDELVGRARISPGAGWNVRDTACLAKSYAQMWLLLYFHRRDLRLMANAICSAVPVNWVPTGRTTWSIHAGGEWMTTEDMLEVWNRVWIEENEWMEDKTPVEKWSDVPYSGKREDIWCGSLVGTRARATWAENIQVAINQVRAIIGDEKYVDYMSSLKRYEDTTLVEDTVL

SEQ ID NO: 19 - DNA, 147 bp, West Nile virus
agtagttcgcctgtgtgagctgacaaacttagtagtgtttgtgaggattaacaacaattaacacagtgcgagctgtttcttggcacgaagatctcgatgtctaagaaaccaggagggcccggtaaaaaccgggctgtcaatatgcta

SEQ ID NO: 20 - DNA, 135 bp, West Nile virus
agtagttcgcctgtgtgagctgacaaacttagtagtgtttgtgaggattaacaacaattaacacagtgcgagctgtttcttggcacgaagatctcgatgtctaagaaaccaggagggcccggtaaaaaccgggct

SEQ ID NO: 21 - DNA, 107 bp, West Nile virus
agcatattgacacctgggatagactaggggatcttctgctctgcacaaccagccacacggcacagtgcgccgacataggtggctggtggtgctagaacacaggatct

SEQ ID NO: 22 - DNA, 96 bp, West Nile virus
acctgggatagactaggggatcttctgctctgcacaaccagccacacggcacagtgcgccgacataggtggctggtggtgctagaacacaggatct

SEQ ID NO: 23 - protein, 618 aa, Saint Louis encephalitis virus
GGALWDVPSPKVYPKCETKPGIYRIMTRGILGTFQAGVGVMHEGVFHTMWHATEGAVLRNGEGRLDPYAGDVRNDLISYGGPWKLSATWDGTEEVQMIAVAPGKPAINVQTTPGVFKTPLGTIGAVTLDFPKGTSGSPIINKKGEIIGLYGNGVLIGQGEYVSGIIQGERTEEPIPDAYNEEMLRKRKLTVLELHPGAGKTRKVLPQIIKDCIQKRLRTAVLAPTRVVACEIAEALKGLPIRYLTPAVRNEHQGNEIVDVMCHATLTQKLLTPTRVPNYQVYIMDEAHFIDPASIAARGYISTKVELGEAAAIFMTATPPGTNDPFPDSNSPILDVEAQVPDKAWSTGYEWITNFTGRTVWFVPSVKSGNEIAICLQKAGKRVIQLNRKSFDTEYPKTKNNEWDFVVTTDISEMGANFGAHRVIDSRKCVKPVILEDDDRVILNGPMAITSASAAQRRGRIGRNPSQIGDEYHYGGATNEDDHDLANWTEAKILLDNIYLPNGLVAQMYQPERDKVFTMDGEFRLRGEERKNFVELMRNGDLPVWLAYKVASNGHSYQDRSWCFTGQTNNTILEDNNEVEVFTKTGDRKILRPKWMDARVCCDYQALKSFKEFAAGKR

SEQ ID NO: 24 - protein, 640 aa, Saint Louis encephalitis virus
EEDVNLGSGTRSVGKLTEKPDPRKVGERIRRLREEYQQTWTYDHNNPYRTWNYHGSYEVKPTGSASSMVNGVVRLLSKPWDMITNVTTMAMTDTTPFGQQRVFKEKVDTKAPEPPLGVAQIMDVTTDWLWDFVAREKKPRVCTPEEFKAKVNSHAALGAMFEEQNQWSSAREAVEDPKFWEMVDEEREAHLKGECHTCIYNMMGKREKKTGEFGKAKGSRAIWYMWLGARFLEFEALGFLNEDHWMSRENSYGGVEGKGLQKLGYILQEISQIPGGKMYADDTAGWDTRITKEDLKNEAKITKRMEERHRKLAEAIIDLTYRHKVVKVMRPGPDGKTYMDVISREDQRGSGQVVTYALNTFTNLAVQLIRCMEAEGVVDEDDITRVRLGRLAKAVEWLRKNGPERLSRMAVSGDDCVVKPIDDRFATALHFLNNMSKIRKDIQEWKPSTGWHNWQEVPFCSHHFNELMLKDGRTIVVPCRSQDELIGRARISPGAGWNVKETACLSKSYAQMWLLMYFHRRDLRMMANAICSAVPVNWVPTGRTTWSIHGKGEWMTTEDMLSVWNRVWIEENEYMKDKTPLAAWNDIPYLGKREDIWCGSLIGTRTRATWAENIYAPIMQIRNLIGEEEYRDYMVAQNRF

SEQ ID NO: 25 - DNA, 148 bp, Saint Louis encephalitis virus
agatgttcgcgtcggtgagcggagaggaaacagatttcctttttggaggataataacttaacttgactgcgaacagttttttagcagggaattacccaatgtctaaaaaaccaggaaaacccggtagaaaccgggttgtcaatatgct

SEQ ID NO: 26 - DNA, 137 bp, Saint Louis encephalitis virus
agatgttcgcgtcggtgagcggagaggaaacagatttcctttttggaggataataacttaacttgactgcgaacagttttttagcagggaattacccaatgtctaaaaaaccaggaaaacccggtagaaaccgggtt

SEQ ID NO: 27 - DNA, 107 bp, Saint Louis encephalitis virus
agcatattgacacctggaaagacaggagatcccctgctttttcaacaccagccacaaggcacagagcgccgtaaagtgtggctggtggtgaaaaaatcacaggatct

SEQ ID NO: 28 - DNA, 96 bp, Saint Louis encephalitis virus
acctggaaagacaggagatcccctgctttttcaacaccagccacaaggcacagagcgccgtaaagtgtggctggtggtgaaaaaatcacaggatct

SEQ ID NO: 29 - protein, 649 aa, Yellow fever virus
TLEADVILPIGTRSVETDKGPLDRAAIEERVERIKSEYTATWFHDNDNPYRTWHYCGSYVTRTSGSAASMINGVIKILTYPWDRIEEVTRMAMTDTTPFGQQRVFKEKVDTRAKDPPAGTRKIMKVVNRWLFRHLAREKNPRLCTKEEFIAKVRSHAAIGAFLEEQEQWKTANEAVQDPKFWELVDEERRLHQQGRCRTCVYNMMGKREKKLSEFGKAKGSRAIWYMWLGARYLEFEALGFLNEDHWASRENSGGGVEGIGLQYLGYVIRDLATLEGGGFYADDTAGWDTRITEADLDDEQEILNYMSPHHRKLALAVMEMTYKNKVVKVLRPAPGGKAYMDVISRRDQRGSGQVVTYALNTITNLKVQLIRMAEAEMVIHHQHVQDCDDTVLTKLEAWLTEHGCDRLKRMAVSGDDCVVRPIDDRFGLALSHLNAMSKVRKDISEWQPSKGWDDWENVPFCSHHFHELQLKDGRRIVVPCRDQDELVGRGRVSPGNGWMIKETACLSKAYANMWSLMYFHKRDMRLLSLAVSSAVPTSWVPQGRTTWSVHGKGEWMTTEDMLEVWNRVWITNNPHMQDKTTVKEWRDIPYLTKRQDKLCGSLIGMTNRATWASHIHLVIHRIRTLIGKERYTDYLTVMDRYSVDADLQ

SEQ ID NO: 30 - DNA, 165 bp, Yellow fever virus
agtaaatcctgtgtgctaattgaggtgcattggtctgcaaatcgagttgctaggcaataaacacatttggattaattttaatcgttcgttgagcgattagcagagaactgaccagaacatgtctggtcgtaaagctcagggaaaaaccctgggcgtcaatatggt

SEQ ID NO: 31 - DNA, 146 bp, Yellow fever virus
agtaaatcctgtgtgctaattgaggtgcattggtctgcaaatcgagttgctaggcaataaacacatttggattaattttaatcgttcgttgagcgattagcagagaactgaccagaacatgtctggtcgtaaagctcagggaaaaa

SEQ ID NO: 32 - DNA, 114 bp, Yellow fever virus
accatattgacgccagggaaagaccggagtggttctctgcttttcctccagaggtctgtgagcacagtttgctcaagaataagcagacctttggatgacaaacacaaaaccact

SEQ ID NO: 33 - DNA, 96 bp, Yellow fever virus
aaagaccggagtggttctctgcttttcctccagaggtctgtgagcacagtttgctcaagaataagcagacctttggatgacaaacacaaaaccact

SEQ ID NO: 34 - protein, 640 aa, Murray Valley encephalitis virus
EEDVNLGSGTRAVGKGVQHTDYKRIKSRIEKLKEEYAATWHTDDNHPYRTWTYHGSYEVKPSGSASTLVNGVVRLLSKPWDAITGVTTMAMTDTTPFGQQRVFKEKVDTKAPEPPQGVKTVMDETTNWLWAYLARNKKARLCTREEFVKKVNSHAALGAMFEEQNQWKNAREAVEDPKFWEMVDEERECHLRGECRTCIYNMMGKREKKPGEFGKAKGSRAIWFMWLGARFLEFEALGFLNEDHWMSRENSGGGVEGAGIQKLGYILRDVAQKPGGKIYADDTAGWDTRITQADLENEAKVLELMEGEQRTLARAIIELTYRHKVVKVMRPAAGGKTVMDVISREDQRGSGQVVTYALNTFTNIAVQLVRLMEAEAVIGPDDIESIERKKKFAVRTWLFENAEERVQRMAVSGDDCVVKPLDDRFSTALHFLNAMSKVRKDIQEWKPSQGWYDWQQVPFCSNHFQEVIMKDGRTLVVPCRGQDELIGRARISPGSGWNVRDTACLAKAYAQMWLVLYFHRRDLRLMANAICSSVPVDWVPTGRTTWSIHGKGEWMTTEDMLSVWNRVWILENEWMEDKTTVSDWTEVPYVGKREDIWCGSLIGTRTRATWAENIYAAINQVRSVIGKEKYVDYVQSLRRY

SEQ ID NO: 35 - DNA, 146 bp, Murray Valley encephalitis virus
agacgttcatctgcgtgagcttccgatctcagtattgtttggaaggatcattgattaacgcggtttgaacagttttttggagcttttgatttcaaatgtctaaaaaaccaggaggacccgggaagccccgggtcgtcaatatgcta

SEQ ID NO: 36 - DNA, 134 bp, Murray Valley encephalitis virus
agacgttcatctgcgtgagcttccgatctcagtattgtttggaaggatcattgattaacgcggtttgaacagttttttggagcttttgatttcaaatgtctaaaaaaccaggaggacccgggaagccccgggtc

SEQ ID NO: 37 - DNA, 113 bp, Murray Valley encephalitis virus
agcatattgacacctgggaaaagactaggagatcttctgctctattccaacatcagtcacaaggcaccgagcgccgaacactgtgactgatgggggagaagaccacaggatct

SEQ ID NO: 38 - DNA, 102 bp, Murray Valley encephalitis virus
acctgggaaaagactaggagatcttctgctctattccaacatcagtcacaaggcaccgagcgccgaacactgtgactgatgggggagaagaccacaggatct

SEQ ID NO: 39 - DNA, 591 bp, Artificial Sequence
  source: /note=“Description of Artificial Sequence Synthetic polynucleotide”
gcggccgcgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggcca

SEQ ID NO: 40 - protein, 21 aa, Artificial Sequence
  source: /note=“Description of Artificial Sequence Synthetic peptide”
  VARIANT (1)..(3): /replace=“ ”
  SITE (1)..(21): /note=“Variant residues given in the sequence have no preference with respect to those in the annotations for variant positions”
GSGEGRGSLLTCGDVEENPGP

SEQ ID NO: 41 - protein, 22 aa, Artificial Sequence
  source: /note=“Description of Artificial Sequence Synthetic peptide”
  VARIANT (1)..(3): /replace=“ ”
  SITE (1)..(22): /note=“Variant residues given in the sequence have no preference with respect to those in the annotations for variant positions”
GSGATNFSLLKQAGDVEENPGP

SEQ ID NO: 42 - protein, 23 aa, Artificial Sequence
  source: /note=“Description of Artificial Sequence Synthetic peptide”
  VARIANT (1)..(3): /replace=“ ”
  SITE (1)..(23): /note=“Variant residues given in the sequence have no preference with respect to those in the annotations for variant positions”
GSGQCTNYALLKLAGDVESNPGP

SEQ ID NO: 43 - protein, 25 aa, Artificial Sequence
  source: /note=“Description of Artificial Sequence Synthetic peptide”
  VARIANT (1)..(3): /replace=“ ”
  SITE (1)..(25): /note=“Variant residues given in the sequence have no preference with respect to those in the annotations for variant positions”
GSGVKQTLNFDLLKLAGDVESNPGP

SEQ ID NO: 44 - protein, 205 aa, Artificial Sequence
  source: /note=“Description of Artificial Sequence Synthetic polypeptide”
ACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 45 - protein, 451 aa, Artificial Sequence
  source: /note=“Description of Artificial Sequence Synthetic polypeptide”
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 46 - protein, 213 aa, Artificial Sequence
  source: /note=“Description of Artificial Sequence Synthetic polypeptide”
QIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 47 - protein, 281 aa, Artificial Sequence
  source: /note=“Description of Artificial Sequence Synthetic polypeptide”
MAMMEVQGGPSLGQTCVLIVIFTVLLQSLCVAVTYVYFTNELKQMQDKYSKSGIACFLKEDDSYWDPNDEESMNSPCWQVKWQLRQLVRKMILRTSEETISTVQEKQQNISPLVRERGPQRVAAHITGTRRRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVG

SEQ ID NO: 48 - protein, 281 aa, Artificial Sequence
  source: /note=“Description of Artificial Sequence Synthetic polypeptide”
MAMMEVQGGPSLGQTCVLIVIFTVLLQSLCVAVTYVYFTNELKQMQDKYSKSGIACFLKEDDSYWDPNDEESMNSPCWQVKWQLRQLVRKMILRTSEETISTVQEKQQNISPLVRERGPQRVAAHITGTRGRSNTLSSPNSKNEKALGRKINSWESSRRGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVG

SEQ ID NO: 49 - protein, 281 aa, Artificial Sequence
  source: /note=“Description of Artificial Sequence Synthetic polypeptide”
MAMMEVQGGPSLGQTCVLIVIFTVLLQSLCVAVTYVYFTNELKQMQDKYSKSGIACFLKEDDSYWDPNDEESMNSPCWQVKWQLRQLVRKMILRTSEETISTVQEKQQNISPLVRERGPQRVAAHITGTRRRSNTLSSPNSKNEKALGIKINSWESSRRGHSFLSNLHLRNGELVIHEKGFYYIYSQTYFRFQEEIKENTKNDKQMVQYIYKYTDYPDPILLMKSARNSCWSKDAEYGLYSIYQGGIFELKENDRIFVSVTNEHLIDMDHEASFFGAFLVG

SEQ ID NO: 50 - protein, 281 aa, Artificial Sequence
  source: /note=“Description of Artificial Sequence Synthetic polypeptide”
MAMMEVQGGPSLGQTCVLIVIFTVLLQSLCVAVTYVYFTNELKQMQDKYSKSGIACFLKEDDSYWDPNDEESMNSPCWQVKWQLRQLVRKMILRTSEETISTVQEKQQNISPLVRERGPQRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMHHEASFFGAFLVG

SEQ ID NO: 51 - protein, 281 aa, Artificial Sequence
  source: /note=“Description of Artificial Sequence Synthetic polypeptide”
MAMMEVQGGPSLGQTCVLIVIFTVLLQSLCVAVTYVYFTNELKQMQDKYSKSGIACFLKEDDSYWDPNDEESMNSPCWQVKWQLRQLVRKMILRTSEETISTVQEKQQNISPLVRERGPQRVAAHITGTRGRSNTLSSPNSKNEKALGRKINSWESSRSGHSFLSNLHLRNGELVIHEKGFYYIYSQTYFRFQERIKENTKNDKQMVQYIYKYTSYPDPILLMKSARNSCWSKDAEYGLYSIYQGGIFELKENDRIFVSVTNEHLIDMHHEASFFGAFLVG

SEQ ID NO: 52 - protein, 238 aa, Artificial Sequence
  source: /note=“Description of Artificial Sequence Synthetic polypeptide”
VQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSGGGGSGGGGSGGGGSIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIK

SEQ ID NO: 53 - protein, 567 aa, Artificial Sequence
  source: /note=“Description of Artificial Sequence Synthetic polypeptide”
MKTLSQAQSKTSSQQFSFTGNSSANVIIGNQKLTINDVARVARNGTLVSLTNNTDILQGIQASCDYINNAVESGEPIYGVTSGFGGMANVAISREQASELQTNLVWFLKTGAGNKLPLADVRAAMLLRANSHMRGASGIRLELIKRMEIFLNAGVTPYVYEFGSIGASGDLVPLSYITGSLIGLDPSFKVDFNGKEMDAPTALRQLNLSPLTLLPKEGLAMMNGTSVMTGIAANCVYDTQILTAIAMGVHALDIQALNGTNQSFHPFIHNSKPHPGQLWAADQMISLLANSQLVRDELDGKHDYRDHELIQDRYSLRCLPQYLGPIVDGISQIAKQIEIEINSVTDNPLIDVDNQASYHGGNFLGQYVGMGMDHLRYYIGLLAKHLDVQIALLASPEFSNGLPPSLLGNRERKVNMGLKGLQICGNSIMPLLTFYGNSIADRFPTHAEQFNQNINSQGYTSATLARRSVDIFQNYVAIALMFGVQAVDLRTYKKTGHYDARACLSPATERLYSAVRHVVGQKPTSDRPYIWNDNEQGLDEHIARISADIAAGGVIVQAVQDILPCLH

SEQ ID NO: 54 - protein, 328 aa, Artificial Sequence
  source: /note=“Description of Artificial Sequence Synthetic polypeptide”
MADKLPNIVILATGGTIAGSAATGTQTTGYKAGALGVDTLINAVPEVKKLANVKGEQFSNMASENMTGDVVLKLSQRVNELLARDDVDGVVITHGTDTVEESAYFLHLTVKSDKPVVFVAAMRPATAISADGPMNLLEAVRVAGDKQSRGRGVMVVLNDRIGSARYITKTNASTLDTFKANEEGYLGVIIGNRIYYQNRIDKLHTTRSVFDVRGLTSLPKVDILYGYQDDPEYLYDAAIQHGVKGIVYAGMGAGSVSVRGIAGMRKAMEKGVVVIRSTRTGNGIVPPDEELPGLVSDSLNPAHARILLMLALTRTSDPKVIQEYFHTY

SEQ ID NO: 55 - protein, 121 aa, Artificial Sequence [sequence omitted]

SEQ ID NO: 56 - protein, 112 aa, Artificial Sequence [sequence omitted]

SEQ ID NO: 57 - protein, 160 aa, Homo sapiens
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 58 - protein, 448 aa, Artificial Sequence [sequence omitted]

SEQ ID NO: 59 - DNA, 10 bp, Unknown
  source: /note=“Description of Unknown Kozak sequence”
gccrccatgg

SEQ ID NO: 60 - DNA, 120 bp, Artificial Sequence
  source: /note=“Description of Artificial Sequence Synthetic polynucleotide”
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa